SEQUENCE LISTING (22 sequences)

SEQ ID NO: 1 - DNA, 17 bp, Artificial Sequence
  Primer
aggaggattacaaaatg

SEQ ID NO: 2 - DNA, 41 bp, Artificial Sequence
  Primer
gctctagaaggaggattacaaaatgactaaacatatcatcg

SEQ ID NO: 3 - DNA, 33 bp, Artificial Sequence
  Primer
ttcctttgcggccgctcacttcctattcttcgc

SEQ ID NO: 4 - DNA, 39 bp, Artificial Sequence
  Primer
gctctagaaggaggtgaataacatgaaaaaggtaattat

SEQ ID NO: 5 - DNA, 40 bp, Artificial Sequence
  Primer
ttcctttgcggccgcccttaacattaactaaatatctgat

SEQ ID NO: 6 - DNA, 44 bp, Artificial Sequence
  Primer
gctctagaaggaggattacaaaatgaaatggttatcacgaatat

SEQ ID NO: 7 - DNA, 33 bp, Artificial Sequence
  Primer
ttcctttgcggccgcccttgatttattgttctt

SEQ ID NO: 8 - DNA, 44 bp, Artificial Sequence
  Primer
gctctagaaggaggattacaaaatgaaaaccatgaaaaaatata

SEQ ID NO: 9 - DNA, 32 bp, Artificial Sequence
  Primer
ttcctttgcggccgcttagtcatgacgttcac

SEQ ID NO: 10 - DNA, 27 bp, Artificial Sequence
  Primer
gctctagaaggaggatgtctatgaaaa

SEQ ID NO: 11 - DNA, 33 bp, Artificial Sequence
  Primer
ttcctttgcggccgctagatactagtagcttga

SEQ ID NO: 12 - DNA, 31 bp, Artificial Sequence
  Primer
gacgcgtcgacatatgcggtgtgaaataccg

SEQ ID NO: 13 - DNA, 26 bp, Artificial Sequence
  Primer
gacgcgcatgcccgactggaaagcgg

SEQ ID NO: 14 - DNA, 16 bp, Artificial Sequence
  Primer
ccgactggaaagcggg

SEQ ID NO: 15 - DNA, 15 bp, Artificial Sequence
  Primer
acaagcccgtcaggg

SEQ ID NO: 16 - protein, 497 aa, Staphylococcus aureus
MTKHIIVIGGGLGGISAAIRMAQSGYSVSLYEQNNHIGGKVNRHESDGFGFDLGPSILTMPYIFEKLFEYSKKQMSDYVTIKRLPHQWRSFFPDGTTIDLYEGIKETGQHNAILSKQDIEELQNYLNYTRRIDRITEKGYFNYGLDTLSQIIKFHGPLNALINYDYVHTMQQAIDKRISNPYLRQMLGYFIKYVGSSSYDAPAVLSMLFHMQQEQGLWYVEGGIHHLANALEKLAREEGVTIHTGARVDNIKTYQRRVTGVRLDTGEFVKADYIISNMEVIPTYKYLIHLDTQRLNKLEREFEPASSGYVMHLGVACQYPQLAHHNFFFTENAYLNYQQVFHEKVLPDDPTIYLVNTNKTDHTQAPVGYENIKVLPHIPYIQDQPFTTEDYAKFRDKILDKLEKMGLTDLRKHIIYEDVWTPEDIEKNYRSNRGAIYGFVADKKKNKGFKFPKESQYFENLYFVGGSVNPGGGIPMVTLSGQQVADKINAREAKNRK

SEQ ID NO: 17 - DNA, 1494 bp, Staphylococcus aureus
atgactaaacatatcatcgttattggtggtggcttaggtgggatttctgcagcaattcgaatggcacaaagtggctattcggtctcattatatgaacaaaatactcatataggaggcaaagtgaatcgtcatgaatcagatggctttggctttgatttaggtccatctattttaacgatgccttatatttttgaaaaattattcgaatatagcaagaagcaaatgtcagactacgttacaatcaagcgtttgccacatcaatggcgtagcttttttccagatggcacgactatcgatttgtatgaaggtattaaagaaacaggtcagcataatgcgatattgtcgaaacaggatatagaggaactgcaaaattatttgaattatacaagacgaatcgatcgtattactgaaaaagggtattttaactatggtttagatacactatctcaaattattaaatttcatgggccattaaatgctcttattaattatgattatgtacatactatgcaacaggccatagacaagcgtatctcgaatccatacttgcgacaaatgttaggctattttatcaaatatgtaggttcttcatcatacgatgcgccagctgtattatctatgttattccatatgcaacaagagcaaggcctttggtatgtagaaggtggaatccatcatttagccaatgccttggaaaagctagcgcgtgaagaaggtgtcacaattcatacaggtgcacgtgtggacaatattaaaacatatcaaagacgtgtgacgggtgtcagattagatacaggtgagtttgtaaaggcagattatattatttcaaatatggaagtcatacctacttataaatatttaattcaccttgatactcaacgattaaacaaattagagagggaatttgagccggcaagctcaggatatgtgatgcatttaggtgttgcttgccaatacccgcaattagcacatcataatttcttttttacggaaaatgcttatctcaattatcaacaagtttttcatgaaaaggtattgccagatgatccgaccatttatctagtaaatacgaataaaactgatcacacacaagcgccagtaggttatgaaaatatcaaagtcttaccacatattccatatattcaagatcagccttttaccactgaagattatgcgaagtttagggataaaattttggataaattagaaaaaatgggacttactgatttaagaaaacacattatttatgaagatgtttggacaccggaggatattgaaaaaaattatcgttctaatcgtggtgcaatatatggtgttgtagcagataaaaagaaaaacaaaggatttaaatttcctaaagaaagtcagtattttgaaaacttgtactttgtaggtggatcagtaaatcctggtggtggcatgccaatggttacattaagtgggcaacaagtcgcagacaaaataaacgcgcgagaagcgaagaataggaagtga

SEQ ID NO: 18 - protein, 494 aa, Oceanobacillus iheyensis
VKKVIIIGGGLGGLSAAISMAQLGYSVELFEKNHHLGGKLNRLEQDGFGFDLGPSILTMPHIFERLFANSGKRMEDYISIYQLSHEWRSFFTDGTTIDLYNNPDKMLKQNPSLTERDIKEYKSFLQYAKQIDDITTKGYFDKGLDTTWEIIQEHGMIQSLKKFDLTSTMYEGIEKRITNPKLRSMLAYFIKYVGSSPYDAPAVLNMLIYMQHAYGAWYVPGGMHKIAEGLVKLANELDVQIHTNSEVTKLKKDSTGNVIAATLADDSEIKGDIFISNMEVIPTYEKLLMEKSSYIKKLTKKYEPSSSGLVLHLGVKNSYPQLSHHNFFFSHNLKEQMNQVFHKHQLPDDPTIYLVNTNKTDPNQVPGPGYENIKILPHIPYIQDKPFSDDDYKQFREQVLIKLENMGMHGLRESIVTEDMWTPNDIQSTYYSYKGSIYGTLSNKKINRGFKHPKQSSKYDNLFFVGGSVNPGGGMPMVVLSGQQVSEKIHQIFS

SEQ ID NO: 19 - protein, 480 aa, Exiguobacterium sp.
MSAAIRLAGDGYEVTMLEQNGNVGGKLNQRSGQGFTFDTGPSILTMPWVLEQLFTSVHRRLDDYLEIERIEPQWRTFFEDGTQLDVKGDLPGMLEEFKKVSDQADFVELFSYSKKMYDLCLDSFYKYSLEDLKDLKKYHTMSELLKMDPLNTVASGTKKHLNNKYLEQLFNYMVMYVGSNPYEAPAVFNQMIYVQMGLGIYYVKGGMYNIARAMKTVLDELRVTIHVNTPVARVVTEGKRAIGVETTDGTFYPADVVVSNLEVIPTYQHLISEKKGPKQAKKLNQSFVPSVSGLVLLLGVNREYKDLKHHNFFFSDDPEREFAQMFKDGVPPEDPTIYVGVSSKSDASQAPEGKDNLFVLTHVPPLTKQDGKTDWDAYREVVLDKLERMGLTDLRESIEFEYRFTPEDLKSLYGPNGGSIYGVAADRKKNGGFKIPSKSDLYEGLYFVGGSTHPGGGVPMVTLSGQLTADLIQKHEKVKA

SEQ ID NO: 20 - protein, 497 aa, Staphylococcus aureus; identical to SEQ ID NO: 16

SEQ ID NO: 21 - protein, 494 aa, Oceanobacillus iheyensis; identical to SEQ ID NO: 18

SEQ ID NO: 22 - protein, 480 aa, Exiguobacterium sp.; identical to SEQ ID NO: 19

(19) United States
(12) Patent Application Publication
(10) Pub. No.: US 2009/0176287 A1
(43) Pub. Date: Jul. 9, 2009

(54) PRODUCING CAROTENOIDS

(75) Inventors: Claudia Schmidt-Dannert, Shoreview, MN (US); Benjamin N. Mijts, Belmont, CA (US); Pyung Cheon Lee, St. Paul, MN (US)

Correspondence Address: FISH & RICHARDSON P.C., PO BOX 1022, MINNEAPOLIS, MN, 55440-1022, US

(73) Assignee: REGENTS OF THE UNIVERSITY OF MINNESOTA

(21) Appl. No.: 11/817,120

(22) PCT Filed: Feb. 24, 2006
(86) PCT No.: PCT/US06/06793

§ 371 Date: Nov. 17, 2008

Related U.S. Application Data
(60) Provisional application No. 60/656,110, filed on Feb. 24, 2005.

(51) Int. Cl. C12P 7/24; C12N 1/00; C12N 1/21; C07C 47/02; C07H 21/00

(52) U.S. Cl. 435/147; 435/243; 435/252.33; 435/252.3; 568/448; 536/23.2

(57) ABSTRACT

This document provides methods and materials related to the production of carotenoids. For example, microorganisms containing one or more exogenous nucleic acids and producing detectable amounts of carotenoids are provided.

### STATEMENT AS TO FEDERALLY SPONSORED RESEARCH

Funding for the work described herein was provided by the federal government, which may have certain rights in the invention.

### BACKGROUND

1. Technical Field

This document relates to methods and materials involved in producing carotenoids such as oxygenated carotenoids and acyclic carotenoids.

2. Background Information

Carotenoids form a group of pigmented biomolecules of emerging importance as food supplements or colorants and in nutraceutical and pharmaceutical applications. Carotenoids are structurally classified based on the number of backbone carbon molecules, usually C30, C40, or C50. Carotenoid biosynthesis occurs via a head to head condensation reaction of isoprenoid precursors followed by a desaturation reaction to increase the number of conjugated double bonds generating the distinctive carotenoid chromophore. Generally, well-conserved carotenoid synthase and desaturase enzymes catalyze these reactions (Mijts et al., Methods Enzymol., 388: 315-29 (2004)).

### SUMMARY

This document provides methods and materials related to metabolic pathways with a functionally diverse array of modifying enzymes to engineer pathways for the recombinant production of carotenoid structures in microorganisms. These methods and materials can be used to obtain carotenoids such as naturally-occurring carotenoids or carotenoids not found in nature. Examples of carotenoids include, without limitation, dialdehyde 2,4,2′,4′-tetradehydrolycopendial; 2,4-didehydrolycopenal; and 2,4,2′,4′-tetradehydrolycopenal. Genes located later in a biosynthetic pathway can be modified and can exhibit a higher catalytic promiscuity than those earlier in the pathway, allowing them to accept unnatural substrates. Using directed evolution to diverge natural pathways towards new possible metabolic routes in combination with an extension of these pathways with additional genes is a powerful approach to discover novel natural and unnatural compounds and produce these compounds in microbial hosts.

In one aspect, the invention features a microorganism that includes an exogenous nucleic acid encoding a diapophytoene synthase, a dehydrosqualene desaturase, and a carotenoid oxygenase, wherein the microorganism produces detectable amounts of a 4,4-diapo-ζ-carotene or a diaponeurosporene or a diapolycopene derivative, the derivative having a terminal aldehyde or terminal carboxyl acid moiety (e.g., diapolycopene dialdehyde or diapolycopene dicarboxylic acid). For example, the derivative can be 4,4′-diapo-ζ-carotene-al or 4,4′-diapo-ζ-carotene dial. The derivative also can be a water soluble carotenoid such as norbixin or a norbixin-like compound. The diapophytoene synthase can be the S. aureus or O. iheyensis diapophytoene synthase. The dehydrosqualene desaturase can be the S. aureus or O. iheyensis dehydrosqualene desaturase. The carotenoid oxygenase can be the S. aureus or O. iheyensis carotenoid oxygenase. The exogenous nucleic acid further can encode a farnesyl diphosphate synthase (e.g., IspA).

The invention also features a microorganism that includes an exogenous nucleic acid encoding a diapophytoene synthase, a diapophytoene desaturase, and a lycopene cyclase, wherein the microorganism produces detectable amounts of diapotorulene. The exogenous nucleic acid further can encode a farnesyl diphosphate synthase. Methods for producing diapotorulene can include culturing such a microorganism under conditions wherein the microorganism produces diapotorulene.

In another aspect, the invention features a microorganism that includes an exogenous nucleic acid encoding a diapophytoene synthase, a diapophytoene desaturase, and a spheroidene monooxygenase, wherein the microorganism produces detectable amounts of an acyclic C35 carotenoid. Methods for producing acyclic C35 carotenoids can include culturing such a microorganism under conditions wherein the microorganism produces the acyclic C35 carotenoids.

Microorganisms that include an exogenous nucleic acid encoding geranyl geranyl diphosphate (GGDP) synthase, phytoene synthase, phytoene desaturase, and a spheroidene monooxygenase also are featured, wherein the microorganism produces detectable amounts of an acyclic xanthophyll or a tetradehydrolycopene derivative. The acyclic xanthophylls can be selected from the group consisting of ζ-carotene-2-one, neurosporene-2-one, and lycopene-2-one. The tetradehydrolycopene derivative can be phillipsiaxanthin. Methods for producing an acyclic xanthophyll or a tetradehydrolycopene derivative can include culturing such a microorganism under conditions wherein the microorganism produces the compound.

In yet another aspect, the invention features a microorganism that includes an exogenous nucleic acid encoding GGDP synthase, phytoene synthase, phytoene desaturase, a lycopene cyclase, and a β-carotene oxygenase, the microorganism producing detectable amounts of ketotorulene. Methods for producing ketotorulene can include culturing such a microorganism under conditions wherein the microorganism produces ketotorulene.

The invention also features a microorganism that includes an exogenous nucleic acid encoding GGDP synthase, phytoene synthase, phytoene desaturase, a lycopene cyclase, and a β-carotene desaturase, the microorganism producing detectable amounts of didehydro-βφ-carotene. Methods for producing didehydro-βφ-carotene can include culturing such a microorganism under conditions wherein the microorganism produces didehydro-βφ-carotene.

In another aspect, the invention features a microorganism that includes an exogenous nucleic acid encoding GGDP synthase, phytoene synthase, phytoene desaturase, a lycopene cyclase, and a β-carotene hydroxylase, the microorganism producing detectable amounts of hydroxytorulene. The exogenous nucleic acid further can encode a zeaxanthin glucosylase such that the microorganism produces detectable amounts of torulene glucoside. Methods for producing torulene glucoside can include culturing such a microorganism under conditions wherein the microorganism produces torulene glucoside.

In yet another aspect, the invention features a composition that includes one or more compounds selected from the group consisting of diapolycopene dialdehyde, diapolycopene dicarboxylic acid, diapotorulene, ζ-carotene-2-one, neurosporene-2-one, lycopene-2-one, phillipsiaxanthin, ketotorulene, didehydro-βφ-carotene, hydroxytorulene, and torulene glucoside. The composition can be a food composition.

The invention also features a composition that includes a compound selected from the group consisting of 4,4′-diapo-ζ-carotene-al and 4,4′-diapo-ζ-carotene-dial. The composition can be a food composition.

In another aspect, the invention features a method of making a compound selected from the group consisting of 4,4′-diapo-ζ-carotene-al and 4,4′-diapo-ζ-carotene-dial. The method includes culturing a microorganism that includes an exogenous nucleic acid encoding a diapophytoene synthase, a dehydrosqualene desaturase, and a carotenoid oxygenase under conditions wherein the microorganism produces the compound. The method further can include extracting the compound from the microorganism. The microorganism can produce at least about 1 mg/L, 10 mg/L, or 100 mg/L of the compound.

In another aspect, the invention features a microorganism containing exogenous nucleic acid encoding a polypeptide having a carotenoid oxygenase activity, wherein the microorganism has a geranylgeranyl diphosphate (GGDP) synthase activity, a phytoene synthase activity, and a phytoene desaturase activity and produces detectable amounts of at least one compound selected from the group consisting of dialdehyde 2,4,2′,4′-tetradehydrolycopendial; 2,4-didehydrolycopenal; and 2,4,2′,4′-tetradehydrolycopenal. The polypeptide having the carotenoid oxygenase activity can be an S. aureus carotenoid oxygenase. The polypeptide having the carotenoid oxygenase activity can be an O. iheyensis carotenoid oxygenase. The microorganism can produce more 2,4,2′,4′-tetradehydrolycopendial than lycopene such that the ratio is greater than 3:1 2,4,2′,4′-tetradehydrolycopendial to lycopene. The ratio can be greater than 5:1, greater than 10:1, or greater than 20:1. The polypeptide having the carotenoid oxygenase activity can be crtOx(SA)ₘᵤₜ₁ or crtOx(SA)ₘᵤₜ₂. The polypeptide having the carotenoid oxygenase can be crtOx(SA)ₘᵤₜ₃. The microorganism can contain exogenous nucleic acid encoding a polypeptide having the geranylgeranyl diphosphate synthase activity, a polypeptide having the phytoene synthase activity, and a polypeptide having the phytoene desaturase activity. The polypeptide having the geranylgeranyl diphosphate synthase activity can be an E. uredovora geranylgeranyl diphosphate synthase. The polypeptide having the phytoene synthase activity can be an E. uredovora phytoene synthase. The polypeptide having the phytoene desaturase activity can be an E. uredovora phytoene desaturase. The polypeptide having the phytoene desaturase activity can be crtI₁₄. The exogenous nucleic acid encoding the polypeptide having the geranylgeranyl diphosphate synthase activity, the polypeptide having the phytoene synthase activity, the polypeptide having the phytoene desaturase activity, and the polypeptide having the carotenoid oxygenase activity can be located on a single nucleic acid molecule. The exogenous nucleic acid encoding the polypeptide having carotenoid oxygenase activity can be located on a nucleic acid molecule separate from the exogenous nucleic acid encoding the polypeptide having the geranylgeranyl diphosphate synthase activity, the polypeptide having the phytoene synthase activity, and the polypeptide having the phytoene desaturase activity. The phytoene desaturase activity can be capable of catalyzing production of a fully conjugated 3,4,3′,4′-tetradehydrolycopene. The microorganism can produce detectable amounts of dialdehyde 2,4,2′,4′-tetradehydrolycopendial. The microorganism can produce detectable amounts of 2,4-didehydrolycopenal. The microorganism can produce detectable amounts of 2,4,2′,4′-tetradehydrolycopenal. The microorganism can be E. coli or S. aureus.

In another aspect, the invention features a composition containing a compound selected from the group consisting of dialdehyde 2,4,2′,4′-tetradehydrolycopendial; 2,4-didehydrolycopenal; and 2,4,2′,4′-tetradehydrolycopenal. Greater than 10, 20, 30, 40, 50, 60, 70, 80, 90, or 95 percent of the composition can be the compound. The composition can be a food composition.

In another aspect, the invention features a method of making a compound selected from the group consisting of dialdehyde 2,4,2′,4′-tetradehydrolycopendial; 2,4-didehydrolycopenal; and 2,4,2′,4′-tetradehydrolycopenal. The method includes culturing a microorganism under conditions wherein the microorganism produces the compound. The microorganism contains exogenous nucleic acid encoding a polypeptide having a carotenoid oxygenase activity, wherein the microorganism has a geranylgeranyl diphosphate (GGDP) synthase activity, a phytoene synthase activity, and a phytoene desaturase activity and produces detectable amounts of at least one compound selected from the group consisting of dialdehyde 2,4,2′,4′-tetradehydrolycopendial; 2,4-didehydrolycopenal; and 2,4,2′,4′-tetradehydrolycopenal. The method can include extracting the compound from the microorganism. The microorganism can produce at least about 1 mg/L of the compound. The microorganism can produce at least about 10 mg/L of the compound. The microorganism can produce at least 100 mg/L of the compound.

In another aspect, the invention features an isolated nucleic acid molecule encoding a carotenoid oxygenase that, when expressed in a microorganism having a geranylgeranyl diphosphate synthase activity, a phytoene synthase activity, and a phytoene desaturase activity, results in the microorganism producing more 2,4,2′,4′-tetradehydrolycopendial than lycopene such that the ratio is greater than 3:1 2,4,2′,4′-tetradehydrolycopendial to lycopene. The ratio can be greater than 5:1, 10:1, or 20:1. The isolated nucleic acid molecule can encode crtOx(SA)ₘᵤₜ₁ or crtOx(SA)ₘᵤₜ₂. The isolated nucleic acid molecule can encode crtOx(SA)ₘᵤₜ₃.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is a schematic of biosynthetic routes to different acyclic and cyclic C40 and C30 carotenoids in engineered E. coli. Red arrows indicate branching of the central desaturation pathways to the routes for the biosynthesis of novel carotenoid structures (red).

FIGS. 2A-2C are HPLC analyses of carotenoid extracts of E. coli transformants expressing C30 carotenogenic enzymes (CrtM and CrtN) on pAC-crtM(SA)-crtN(SA) (A) together with lycopene cyclase pUC-crtY(EU) (B) or spheroidene monooxygenase pUC-crtA(RC) (C). The following diapocarotenoids were identified: peak 1, diaponeurosporene (λmax: 415, 438, 467; M+ at m/e=402.2); peak 2, diapolycopene (λmax: 443, 468, 503; M+ at m/e=400.1); peak 3, diapotorulene (λmax: 425, 449; M+ at m/e=402.1); peak 4, diaponeurosporene-derivative (λmax: 399, 422, 449; M+ at m/e=536.3). Double or triple peaks represent different geometrical isomers. Insets: recorded absorption spectra for individual peaks.

FIGS. 2D and 2E are the ESI mass spectra of diapolycopene and diapotorulene, respectively.

FIG. 2F is the APCI mass spectrum of the C35 ketocarotenoid.

FIGS. 3A and 3B are HPLC and HP-TLC analysis of E. coli cells producing acyclic oxygenated C40 carotenoids. HPLC and HP-TLC analysis of carotenoid extracts of E. coli pAC-crtE(EU)-crtB(EU)-crtI(EU) (A) and E. coli pAC-crtE(EU)-crtB(EU)-crtI₁₄ (B) both coexpressing spheroidene monooxygenase (pUC-crtA(RC)). The following carotenoids were identified: peak 1, ζ-carotene (λmax: 377, 400, 424; M+ at m/e=540.4); peak 2, neurosporene (λmax: 419, 442, 470; M+ at m/e=538.4); peak 3, lycopene (λmax: 449, 475, 507; M+ at m/e=536.4); peak 4, ζ-carotene-2-one (λmax: 377, 400, 424; M+ at m/e=556.4); peak 5, neurosporene-2-one (λmax: 419, 442, 470; M+ at m/e=554.4); peak 6, lycopene-2-one (λmax: 449, 475, 507; M+ at m/e=552.4); peak 7, phillipsiaxanthin (λmax: 516, 524; M+ at m/e=596.3). Double or triple peaks represent different geometrical isomers. Insets: recorded absorption spectra for individual peaks.

FIGS. 3C-3E are ESI mass spectra of ζ-carotene-2-one, neurosporene-2-one, and lycopene-2-one, respectively.

FIG. 3F is the APCI mass spectrum of phillipsiaxanthin.

FIGS. 4A-4F are HPLC analyses of carotenoid extracts of E. coli transformants expressing: (A) pAC-crtE(EU)-crtB(EU)-crtI₁₄-crtY(EU) (β,β-carotene pathway); (B) pAC-crtE(EU)-crtB(EU)-crtI₁₄-crtY2(EU/EH) (evolved torulene pathway); (C) pAC-crtE(EU)-crtB(EU)-crtI₁₄-crtY(EU); and (D) pAC-crtE(EU)-crtB(EU)-crtI₁₄-crtY2(EU/EH), extended with carotene oxygenase CrtO on pUC-crtO(SY); and (E) pAC-crtE(EU)-crtB(EU)-crtI₁₄-crtY(EU) and (F) pAC-crtE(EU)-crtB(EU)-crtI₁₄-crtY2(EU/EH), extended with carotene desaturase CrtU on pUC-crtU(BL). The following carotenoids were identified: peak 1, β,β-carotene (λmax: 425, 451, 478; M+ at m/e=536.4); peak 2, torulene (λmax: 454, 481, 514; M+ at m/e=534.4); peak 3, lycopene (λmax: 449, 475, 507; M+ at m/e=536.4); peak 4, echinenone (λmax: 457; M+ at m/e=550.4); peak 5, canthaxanthin (λmax: 463; M+ at m/e=564.4); peak 6, Ketotorulene (λmax: 454, 481, 514; M+ at m/e=548.3); peak 7, isoreniaratene (λmax: 425, 451, 478; M+ at m/e=528.3); peak 8, didehydro-β,φ-carotene (λmax: 454, 481, 514; M+ at m/e=530.2). Double or triple peaks represent different geometrical isomers. Insets: recorded absorption spectra for individual peaks.

FIG. 4G is the ESI mass spectrum of 4-keto-torulene.

FIG. 4H is the APCI mass spectrum of didehydro-β,φ-carotene.

FIGS. 5A-5D are HPLC analyses of carotenoid extracts of E. coli cells carrying: (A) pAC-crtE(EU)-crtB(EU)-crtI₁₄-crtY(EU) (β,β-carotene pathway) and (B) pAC-crtE(EU)-crtB(EU)-crtI₁₄-crtY2(EU/EH) (evolved torulene pathway), together with β-carotene hydroxylase (crtZ); and (C) pAC-crtE(EU)-crtB(EU)-crtI₁₄-crtY(EU)-crtZ(EH) and (D) pAC-crtE(EU)-crtB(EU)-crtI₁₄-crtY2(EU/EH)-crtZ(EH), together with zeaxanthin glucosylase (crtX). The following carotenoids were identified: peak 1, zeaxanthin (λmax: 425, 451, 478; M+ at m/e=568.3); peak 2, hydroxy-torulene (λmax: 454, 481, 514; M+ at m/e=550.3); peak 3, β-cryptoxanthin-monoglucoside (λmax: 425, 451, 478; M+ at m/e=714.5); peak 4, zeaxanthin-monoglucoside (λmax: 425, 451, 478; M+ at n/e=730.5); peak 5, zeaxanthin-diglucoside (λmax: 425, 451, 478; M+ at m/e=892.5); peak 6, torulene-monoglucoside (λmax: 454, 481, 514; M+ at m/e=712.4).

FIGS. 5E and 5F are the ESI mass spectra of hydroxytorulene and torulene glucoside, respectively.

FIG. 6 is a schematic of the subcloning of carotenoid genes required for lycopene production from pUC-crtE(EU), pUC-crtB(EU), pUC-crtI(EU) into pGAPZ.

FIG. 7 is a schematic of the assembly of a tri-gene construct in pGAPZ for lycopene production in P. pastoris.

FIG. 8 is an HPLC-analysis of a carotenoid extract obtained from lycopene producing engineered P. pastoris transformants overexpressing genes crtE, crtB, and crtI.

FIG. 9 is a biosynthetic pathway leading to the production of novel purple C30 carotenoids in engineered E. coli cells.

FIG. 10 depicts the analysis of purple carotenoid extracts from E. coli cells co-expressing crtM and crtN with a carotenoid oxygenase.

FIG. 11A and FIG. 11B are schematics of the Staphylococcus aureus and Oceanobacillus iheyensis, respectively, carotenoid operon maps.

FIG. 12 is a diagram of the C30 biosynthetic pathway using CrtOx. Overproduced and identified purple carotenoid structures are boxed.

FIG. 13 is flow chart of biosynthetic pathways to generate oxygenated, linear C30 (A) and C40 (B) carotenoids. Solid arrows represent natural biosynthetic pathways suggested for staphyloxanthin in S. aureus (A) and lycopene (B). Biosynthetic pathway steps in engineered recombinant E. coli are indicated by dashed arrows.

FIG. 14 contains photographs of LB media cultures (top), cell pellets (center) and TLC analysis (bottom) of recombinant C30 (A) and C40 (B) carotenoid producing E. coli strains. Background plasmids strains are JM109 pAC-ispA(EC))-crtM(SA)-crtN(SA) (A) and pAC-crtE(EU)-crtB(EU)-crtI₁₄ (B) co-transformed with 1. pUCMod, 2. pUC-crtOx(SA), 3. pUC-crtOx(SA)ₘᵤₜ₁ 4. pUC-crtOx(SA)ₘᵤₜ₂ 5. pUC-crtOx(SA)ₘᵤₜ₃. Identified compounds from FIG. 13 are indicated.

FIG. 15 contains HPLC profiles of recombinant E. coli expressing the C30 carotenoid background plasmid pAC-ispA(EC)-crtM(SA)-crtN(SA) with (A) pUC-crtOx(SA), (B) pUC-crtOx(SA)ₘᵤₜ₁ (C) pUC-crtOx(SA)ₘᵤₜ₂ or (D) pUC-crtOx(SA)ₘᵤₜ₃.

FIG. 16 is a UV-Vis scan of pigment remaining after solvent extraction. E. coli strain JM109 harboring pAC-ispA(EC)-crtM(SA)-crtN(SA) and pUC-crtOx(SA) was cultured for 24 hours in LB glycerol medium, solvent accessible carotenoids were completely extracted from cell pellets with acetone, and the remaining pigment solubilized in 1% KOH for 2 hours at room temperature.

FIG. 17 contains HPLC profiles at wavelengths of 300 nm and 500 nm of recombinant E. coli expressing the C40 carotenoid lycopene background plasmid pAC-crtE(EU)-crtB(EU)-crtI(EU) with (A) pUCMod (B) pUC-crtOx(SA). Based on mass spectrometry, HPLC retention time, and UV-Vis spectra, the peaks were identified as L: lycopene (500 nm) and P: phytoene (300 nm).

FIG. 18 contains HPLC profiles of recombinant E. coli expressing the C40 carotenoid background plasmid pAC-crtE(EU)-crtB(EU)-crtI₁₄ with (A) pUC-crtOx(SA), (3) pUC-crtOx(SA)ₘᵤₜ₁, (C) pUC-crtOx(SA)ₘᵤₜ₂, or (D) pUC-crtOx(SA)ₘᵤₜ₃.

FIG. 19 is a schematic diagram of the CrtOx polypeptide and the amino acid changes observed in mutants CrtOx(SA)ₘᵤₜ₁, CrtOx(SA)ₘᵤₜ₂, and CrtOx(SA)ₘᵤₜ₃.

FIG. 20 contains a sequence listing of the amino acid (SEQ ID NO:16) and nucleic acid (SEQ ID NO:17) sequence of CrtOx from S. aureus strain Mu50.

FIG. 21 contains a sequence listing of the amino acid (SEQ ID NO:18) sequence of CrtOx from O. iheyensis strain HTE 831.

FIG. 22 contains a sequence listing of the amino acid (SEQ ID NO:19) sequence of CrtOx from Exiguobacterium sp. 255-15.

FIG. 23 contains a sequence alignment of CrtOx polypeptides from S. aureus strain Mu50 (SEQ ID NO:20), O. iheyensis strain HTE 831 (SEQ ID NO:21), and Exiguobacterium sp. 255-15 (SEQ ID NO:22).

### DETAILED DESCRIPTION

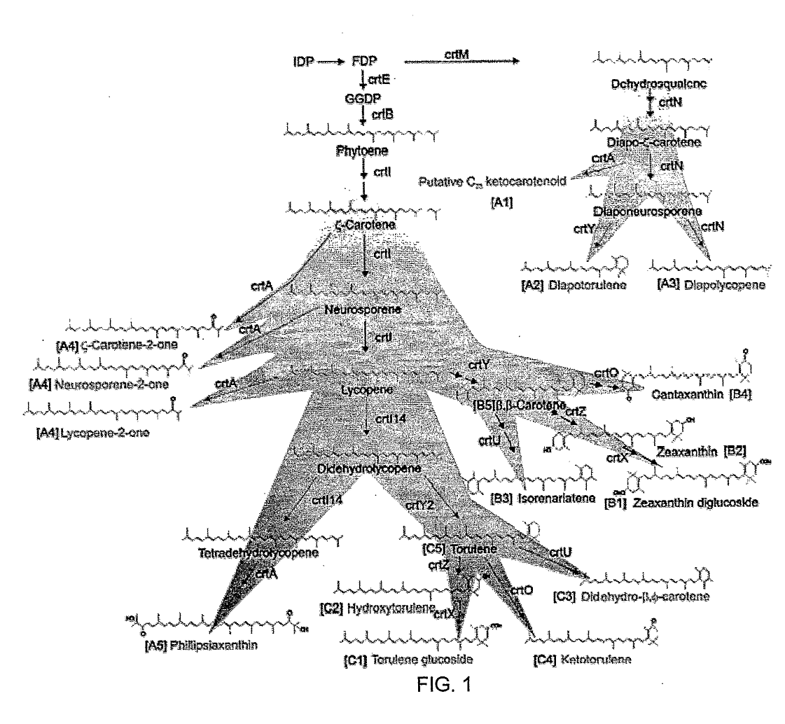

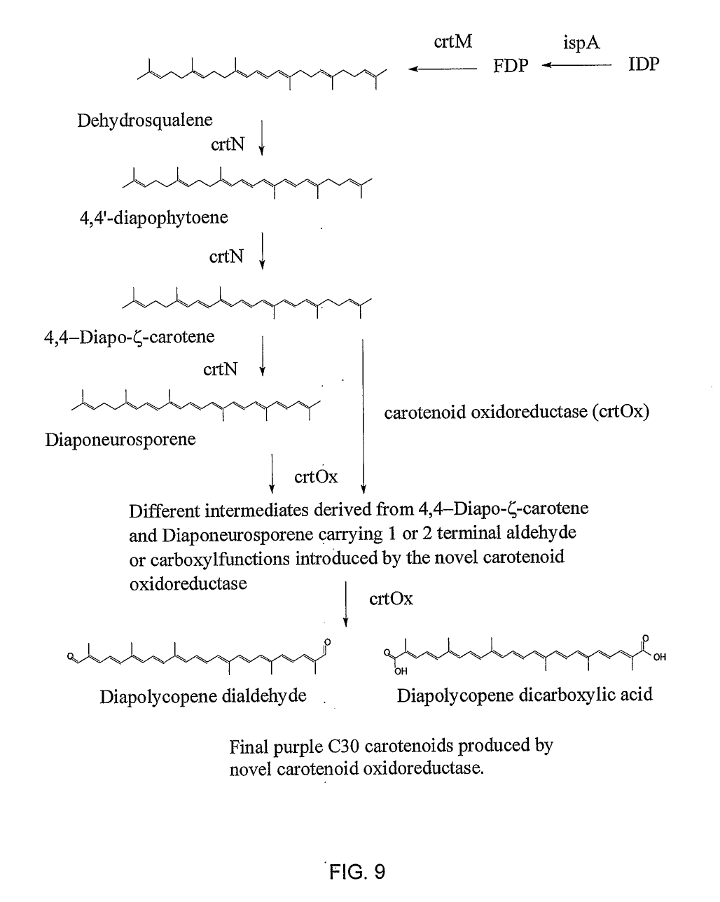

In general, this document provides methods and materials for producing carotenoids in microorganisms. The first committed step in C40 carotenoid biosynthesis is the extension of the general isoprenoid pathway by the enzymes geranyl geranyl disphosphate (GGDP) synthase (CrtE) and phytoene synthase (CrtB) to form the colorless carotenoid phytoene. The introduction of additional double bonds into phytoene by phytoene desaturase (CrtI) produces the colored carotenoids neurosporene (three desaturations) or lycopene (four desaturations) from which different acyclic and cyclic carotenoids are then synthesized (FIG. 1). C30 carotenoid biosynthesis also is an extension of the general isoprenoid pathway by the enzyme dehydrosqualene synthase (CrtM) to form dehydrosqualene (FIGS. 1 and 9). Diapophytoene synthase (CrtN) can desaturate dehydrosqualene to form various carotenoids, including 4,4′-diapophytoene, 4,4-diapo-ζ-carotene, and diaponeurosporene. Carotenoid oxidoreductase (CrtOx) (also called carotenoid oxidase herein) can introduce terminal aldehyde or carboxy functions into 4,4-diapo-ζ-carotene and diaponeurosporene.

Fully conjugated C30 carotenoids containing terminal oxygen functional groups at their acylic end groups are useful, for example, as food colorants (e.g., as a substitute for annatto, which is extracted from the plant Bixa orella) as well as building blocks for self-assembled vesicles for drig-delivery and conducting polymers. For example, the lipase of Candida antartica can be used to synthesize polymers from carotenoid dicarboxylic acids and alcohols such as glycerol or other diols. Carotenoids that contain polar oxygen groups on both ends also can be used to form unilamellar vesicles in which the membrane spanning carotenoid molecule is in contact with both the hydrophilic exterior and interior of the vesicle (as opposed to two phospholipid molecules in biomembranes).

### Microorganisms for Producing Carotenoids

Any microorganism, eukaryotic or prokaryotic, can be used to produce carotenoids, including bacteria (e.g., Escherichia coli, Bacillus, Brevibacterium, Streptomyces, or Pseudomonas), yeast (e.g., Pichia pastoris, Phaffia rhodozyina, or Saccharomyces cerevisiae) and other fungi (e.g., Neurospora crassa), and algae (e.g., Dunaliella sp.). Such microorganisms may or may not naturally produce carotenoids. Microorganisms that are considered “food grade” (i.e., non-toxigenic) and have the ability to accumulate carotenoids are particularly useful. For example, yeast cells have a diverse isoprenoid metabolism and can accumulate large quantities of ergosterols, lipophilic compounds like carotenoids, in their membranes. P. pastoris, a non-carotenogenic methylotropic yeast is particularly useful as it has extreme peroxisome proliferation ability under inducing conditions. In addition, P. pastoris can be grown to extremely high cell densities (>130 g dry cell weight per liter).

Typically, a microorganism can be genetically modified such that one or more particular carotenoids are produced. Such microorganisms can contain one or more exogenous nucleic acid molecules that encode polypeptides having enzymatic activity. The term “nucleic acid” as used herein encompasses both RNA and DNA, including cDNA, genomic DNA, and synthetic (e.g., chemically synthesized) DNA. The nucleic acid can be double-stranded or single-stranded. Where single-stranded, the nucleic acid can be the sense strand or the antisense strand. In addition, nucleic acid can be circular or linear.

The term “exogenous” as used herein with reference to nucleic acid and a particular microorganism refers to any nucleic acid that does not originate from that particular microorganism as found in nature. Thus, non-naturally-occurring nucleic acid is considered to be exogenous to a microorganism once introduced into the microorganism. It is important to note that non-naturally-occurring nucleic acid can contain nucleic acid sequences or fragments of nucleic acid sequences that are found in nature provided the nucleic acid as a whole does not exist in nature. For example, a nucleic acid molecule containing a genomic DNA sequence within an expression vector is non-naturally-occurring nucleic acid, and thus is exogenous to a microorganism once introduced into the microorganism, since that nucleic acid molecule as a whole (genomic DNA plus vector DNA) does not exist in nature. Thus, any vector, autonomously replicating plasmid, or virus (e.g., retrovirus, adenovirus, or herpes virus) that as a whole does not exist in nature is considered to be non-naturally-occurring nucleic acid. It follows that genomic DNA fragments produced by PCR or restriction endonuclease treatment as well as cDNAs are considered to be non-naturally-occurring nucleic acid since they exist as separate molecules not found in nature. It also follows that any nucleic acid containing a promoter sequence and polypeptide-encoding sequence (e.g., cDNA or genomic DNA) in an arrangement not found in nature is non-naturally-occurring nucleic acid.

Nucleic acid that is naturally-occurring can be exogenous to a particular cell. For example, an entire chromosome isolated from a cell of person X is an exogenous nucleic acid with respect to a cell of person Y once that chromosome is introduced into Y's cell.

It is noted that a microorganism can be given an exogenous nucleic acid molecule that encodes a polypeptide having an enzymatic activity that catalyzes the production of a compound not normally produced by that microorganism. Alternatively, a microorganism can be given an exogenous nucleic acid molecule that encodes a polypeptide having an enzymatic activity that catalyzes the production of a compound that is normally produced by that microorganism. In this case, the genetically modified microorganism can produce more of the compound, or can produce the compound more efficiently, than a similar microorganism not having the genetic modification.

A polypeptide having a particular enzymatic activity can be a polypeptide that is either naturally-occurring or non-naturally-occurring. A naturally-occurring polypeptide is any polypeptide having an amino acid sequence as found in nature, including wild-type and polymorphic polypeptides. Such naturally-occurring polypeptides can be obtained from any species including, without limitation, animal (e.g., mammalian), plant, fungal, and bacterial species. A non-naturally-occurring polypeptide is any polypeptide having an amino acid sequence that is not found in nature. Thus, a non-naturally-occurring polypeptide can be a mutated version of a naturally-occurring polypeptide, or an engineered polypeptide. For example, a non-naturally-occurring polypeptide having dehydrosqualene synthase activity can be a mutated version of a naturally-occurring polypeptide having dehydrosqualene synthase activity that retains at least some dehydrosqualene synthase activity. A polypeptide can be mutated by, for example, sequence additions, deletions, substitutions, or combinations thereof.

This document provides genetically modified microorganisms that can be used to perform one or more steps of a metabolic pathway described herein. For example, an individual microorganism can contain exogenous nucleic acid such that each of the polypeptides necessary to perform the steps depicted in FIG. 1 or 9 are expressed. It is important to note that such microorganisms can contain any number of exogenous nucleic acid molecules. For example, a particular microorganism can contain three exogenous nucleic acid molecules with each one encoding one of the three polypeptides necessary to convert farnesyl diphosphate (FDP) into a C30 purple carotenoid such as diapolycopene dialdehyde or diapolycopene dicarboxylic acid as depicted in FIG. 9, or a particular microorganism can endogenously produce polypeptides necessary to convert FDP into dehydrosqualene while containing exogenous nucleic acids that encode polypeptides necessary to convert dehydrosqualene into a C30 purple carotenoid.

In addition, a single exogenous nucleic acid molecule can encode one or more than one polypeptide. For example, a single exogenous nucleic acid molecule can contain sequences that encode two or three different polypeptides. Further, the cells described herein can contain a single copy, or multiple copies (e.g., about 5, 10, 20, 35, 50, 75, 100 or 150 copies), of a particular exogenous nucleic acid molecule. Again, the cells described herein can contain more than one particular exogenous nucleic acid molecule. For example, a particular cell can contain about 50 copies of exogenous nucleic acid molecule X as well as about 75 copies of exogenous nucleic acid molecule Y.

A nucleic acid molecule encoding a polypeptide having enzymatic activity can be identified and obtained using any method such as those described herein. For example, nucleic acid molecules that encode a polypeptide having enzymatic activity can be identified and obtained using common molecular cloning or chemical nucleic acid synthesis procedures and techniques, including PCR. In addition, standard nucleic acid sequencing techniques and software programs that translate nucleic acid sequences into amino acid sequences based on the genetic code can be used to determine whether or not a particular nucleic acid has any sequence homology with known enzymatic polypeptides. Sequence alignment software such as MEGALIGN® (DNASTAR, Madison, Wis., 1997) can be used to compare various sequences. In addition, nucleic acid molecules encoding known enzymatic polypeptides can be mutated using common molecular cloning techniques (e.g., site-directed mutagenesis). Possible mutations include, without limitation, deletions, insertions, and base substitutions, as well as combinations of deletions, insertions, and base substitutions. Further, nucleic acid and amino acid databases (e.g., GenBank®) can be used to identify a nucleic acid sequence that encodes a polypeptide having enzymatic activity. Briefly, any amino acid sequence having some homology to a polypeptide having enzymatic activity, or any nucleic acid sequence having some homology to a sequence encoding a polypeptide having enzymatic activity can be used as a query to search GenBank®. The identified polypeptides then can be analyzed to determine whether or not they exhibit enzymatic activity.

In addition, nucleic acid hybridization techniques can be used to identify and obtain a nucleic acid molecule that encodes a polypeptide having enzymatic activity. Such similar nucleic acid molecules then can be isolated, sequenced, and analyzed to determine whether the encoded polypeptide has enzymatic activity. Briefly, any nucleic acid molecule that encodes a known enzymatic polypeptide, or fragment thereof, can be used as a probe to identify a similar nucleic acid molecules by hybridization under conditions of moderate to high stringency. For the purpose of this invention, moderately stringent hybridization conditions mean the hybridization is performed at about 42° C. in a hybridization solution containing 25 mM KPO₄ (pH 7.4), 5×SSC, 5× Denhart's solution, 50 μg/mL denatured, sonicated salmon sperm DNA, 50% formamide, 10% Dextran sulfate, and 1-15 ng/mL probe (about 5×10⁷ cpm/μg), while the washes are performed at about 50° C. with a wash solution containing 2×SSC and 0.1% sodium dodecyl sulfate.

Highly stringent hybridization conditions mean the hybridization is performed at about 42° C. in a hybridization solution containing 25 mM KPO₄ (pH 7.4), 5×SSC, 5× Denhart's solution, 50 μg/mL denatured, sonicated salmon sperm DNA, 50% formamide, 10% Dextran sulfate, and 1-15 ng/mL probe (about 5×10⁷ cpm/μg), while the washes are performed at about 65° C. with a wash solution containing 0.2×SSC and 0.1% sodium dodecyl sulfate.

Hybridization can be done by Southern or Northern analysis to identify a DNA or RNA sequence, respectively, that hybridizes to a probe. The probe can be labeled with a biotin, digoxygenin, an enzyme, or a radioisotope such as ³²P. The DNA or RNA to be analyzed can be electrophoretically separated on an agarose or polyacrylamide gel, transferred to nitrocellulose, nylon, or other suitable membrane, and hybridized with the probe using standard techniques well known in the art such as those described in sections 7.39-7.52 of Sambrook et al., (1989) Molecular Cloning, second edition, Cold Spring harbor Laboratory, Plainview, N.Y. Typically, a probe is at least about 20 nucleotides in length.

Expression cloning techniques also can be used to identify and obtain a nucleic acid molecule that encodes a polypeptide having enzymatic activity. For example, a substrate known to interact with a particular enzymatic polypeptide can be used to screen a phage display library containing that enzymatic polypeptide. Phage display libraries can be generated as described elsewhere (Burritt et al., Anal. Biochem. 238:1-13 (1990)), or can be obtained from commercial suppliers such as Novagen (Madison, Wis.).

Further, polypeptide sequencing techniques can be used to identify and obtain a nucleic acid molecule that encodes a polypeptide having enzymatic activity. For example, a purified polypeptide can be separated by gel electrophoresis, and its amino acid sequence determined by, for example, amino acid microsequencing techniques. Once determined, the amino acid sequence can be used to design degenerate oligonucleotide primers. Degenerate oligonucleotide primers can be used to obtain the nucleic acid encoding the polypeptide by PCR. Once obtained, the nucleic acid can be sequenced, cloned into an appropriate expression vector, and introduced into a microorganism.

Any method can be used to introduce an exogenous nucleic acid molecule into a cell. In fact, many methods for introducing nucleic acid into microorganisms such as bacteria and yeast are well known to those skilled in the art. For example, heat shock, lipofection, electroporation, conjugation, fusion of protoplasts, and biolistic delivery are common methods for introducing nucleic acid into bacteria and yeast cells. See, e.g., Ito et al., J. Bacterol. 153:163-168 (1983); Durrens et al., Curr. Genet. 18:7-12 (1990); and Becker and Guarente, Methods in Enzymology 194:182-187 (1991).

An exogenous nucleic acid molecule contained within a particular microorganism can be maintained within that microorganism in any form. For example, exogenous nucleic acid molecules can be integrated into the genome of the microorganism or maintained in an episomal state. In other words, a microorganism of the invention can be a stable or transient transformant. Again, a microorganism described herein can contain a single copy, or multiple copies (e.g., about 5, 10, 20, 35, 50, 75, 100 or 150 copies), of a particular exogenous nucleic acid molecule as described herein.

Methods for expressing an amino acid sequence from an exogenous nucleic acid molecule are well known to those skilled in the art. Such methods include, without limitation, constructing a nucleic acid such that a regulatory element promotes the expression of a nucleic acid sequence that encodes a polypeptide. Typically, regulatory elements are DNA sequences that regulate the expression of other DNA sequences at the level of transcription. Thus, regulatory elements include, without limitation, promoters, enhancers, and the like. Any type of promoter can be used to express an amino acid sequence from an exogenous nucleic acid molecule. Examples of promoters include, without limitation, constitutive promoters, tissue-specific promoters, and promoters responsive or unresponsive to a particular stimulus (e.g., light, oxygen, chemical concentration, and the like). Moreover, methods for expressing a polypeptide from an exogenous nucleic acid molecule in cells such as bacterial cells and yeast cells are well known to those skilled in the art. For example, nucleic acid constructs that are capable of expressing exogenous polypeptides within E. coli are well known. See, e.g., Sambrook et al., Molecular cloning: a laboratory manual, Cold Spring Harbour Laboratory Press, New York, USA, second edition (1989).

Methods of identifying microorganisms that contain exogenous nucleic acid are well known to those skilled in the art. Such methods include, without limitation, PCR and nucleic acid hybridization techniques such as Northern and Southern analysis. In some cases, immunohisto-chemistry and biochemical techniques can be used to determine if a microorganism contains a particular nucleic acid by detecting the expression of the encoded enzymatic polypeptide encoded by that particular nucleic acid molecule. For example, an antibody having specificity for an encoded enzyme can be used to determine whether or not a particular cell contains that encoded enzyme. Further, biochemical techniques can be used to determine if a cell contains a particular nucleic acid molecule encoding an enzymatic polypeptide by detecting an organic product produced as a result of the expression of the enzymatic polypeptide. For example, detection of 4,4′-diapo-lycopene-dial or 4,4′-diapolycopene-al-oic acid after introduction of one or more exogenous nucleic acids that encode polypeptides having CrtN, CrtM, and CrtOx activity into a microorganism that does not normally express such polypeptides can indicate that that microorganism not only contains the introduced exogenous nucleic acid molecule but also expresses the encoded enzymatic polypeptide from that introduced exogenous nucleic acid molecule. Methods for detecting specific enzymatic activities or the presence of particular organic products are well known to those skilled in the art. For example, the presence of a carotenoid such as 4,4′-diapo-lycopene-dial or 4,4′-diapolycopene-al-oic acid can be determined as described elsewhere for other carotenoids (See, e.g., Lee et al. (2003) Chem. Biol. 10:453-62).

This document also provides isolated nucleic acids molecules. The term “isolated” as used herein with reference to nucleic acid refers to a naturally-occurring nucleic acid that is not immediately contiguous with both of the sequences with which it is immediately contiguous (one on the 5′ end and one on the 3′ end) in the naturally-occurring genome of the organism from which it is derived. For example, an isolated nucleic acid can be, without limitation, a recombinant DNA molecule of any length, provided one of the nucleic acid sequences normally found immediately flanking that recombinant DNA molecule in a naturally-occurring genome is removed or absent. Thus, an isolated nucleic acid includes, without limitation, a recombinant DNA that exists as a separate molecule (e.g., a cDNA or a genomic DNA fragment produced by PCR or restriction endonuclease treatment) independent of other sequences as well as recombinant DNA that is incorporated into a vector, an autonomously replicating plasmid, a virus (e.g., a retrovirus, adenovirus, or herpes virus), or into the genomic DNA of a prokaryote or eukaryote. In addition, an isolated nucleic acid can include a recombinant DNA molecule that is part of a hybrid or fusion nucleic acid sequence.

The term “isolated” as used herein with reference to nucleic acid also includes any non-naturally-occurring nucleic acid since non-naturally-occurring nucleic acid sequences are not found in nature and do not have immediately contiguous sequences in a naturally-occurring genome. For example, non-naturally-occurring nucleic acid such as an engineered nucleic acid is considered to be isolated nucleic acid. Engineered nucleic acid can be made using common molecular cloning or chemical nucleic acid synthesis techniques. Isolated non-naturally-occurring nucleic acid can be independent of other sequences, or incorporated into a vector, an autonomously replicating plasmid, a virus (e.g., a retrovirus, adenovirus, or herpes virus), or the genomic DNA of a prokaryote or eukaryote. In addition, a non-naturally-occurring nucleic acid can include a nucleic acid molecule that is part of a hybrid or fusion nucleic acid sequence.

It will be apparent to those of skill in the art that a nucleic acid existing among hundreds to millions of other nucleic acid molecules within, for example, cDNA or genomic libraries, or gel slices containing a genomic DNA restriction digest is not to be considered an isolated nucleic acid.

In some cases, an isolated nucleic acid can encode one or more of the polypeptides provided herein. For example, an isolated nucleic acid can encode crtE, crtB, crtI, and crtOx polypeptides. In some cases, an isolated nucleic acid provided herein can encode a polypeptide having carotenoid oxygenases activity. Such polypeptides can be wild-type or mutated polypeptides having carotenoid oxygenases activity. For example, isolated nucleic acid molecules can be designed to encode an in vitro-evolved CrtOx mutant polypeptide. A mutant crtOx polypeptide can be obtained such that microorganisms expressing the mutant crtOx polypeptide are capable of producing more 2,4,2′,4′-tetradehydrolycopendial than lycopene (e.g., the ratio of 2,4,2′,4′-tetradehydrolycopendial to lycopene can be greater than 3:1, 4:1, 5:1, 6:1, 7:1, 8:1, 9:1, 10:1; 11:1, 12:1, 13:1, 14:1, 15:1, 16:1, 17:1, 18:1, 19:1, or 20:1). Examples of such carotenoid oxygenases include, without limitation, crtOx(SA)ₘᵤₜ₁, crtOx(SA)ₘᵤₜ₂, and crtOx(SA)ₘᵤₜ₃.

### Production of Acyclic Carotenoids

Acyclic carotenoids can be produced in microorganisms by introducing one or more exogenous nucleic acids into the microorganism. For example, nucleic acids encoding dehydrosqualene synthase (CrtM) and diapophytoene synthase (CrtN) can be used in combination with a nucleic acid encoding a carotenoid oxygenase (also called a carotenoid oxidoreductase herein) to produce derivatives of 4,4-diapo-ζ-carotene or a diaponeurosporene having one or two terminal aldehydes or carboxyl acid moieties (e.g., 4,4′-diapo-lycopene-dial, 4,4′-diapo-ζ-carotene-dial, 4,4′-diapo-lycopene-al-oic acid). Organisms containing such C30 carotenoids with terminal aldehyde and carboxyl functions are purple in color. In some embodiments, a nucleic acid encoding a farnesyldiphosphate synthase (FPP synthase) (e.g., IspA from E. coli) can be used in combination with the nucleic acids encoding CrtM, CrtN, and CrtOx.

Genes encoding CrtM and CrtN have been identified from Staphylococcus aureus and Oceanobacillus iheyensis. The nucleic acid sequences of CrtM and CrtN are available in GenBank under Accession Nos. X73889 for S. aureus and Accession No. NC_{—}004193.1 for O. iheyensis; the amino acid sequences of CrtM and CrtN from S. aureus are available in GenBank under Accession Nos. A55548 and B55548, respectively; the amino acid sequences of CrtM and CrtN from O. iheyensis are available in GenBank under Accession Nos. NP_{—}693381, and NP_{—}693382, respectively.

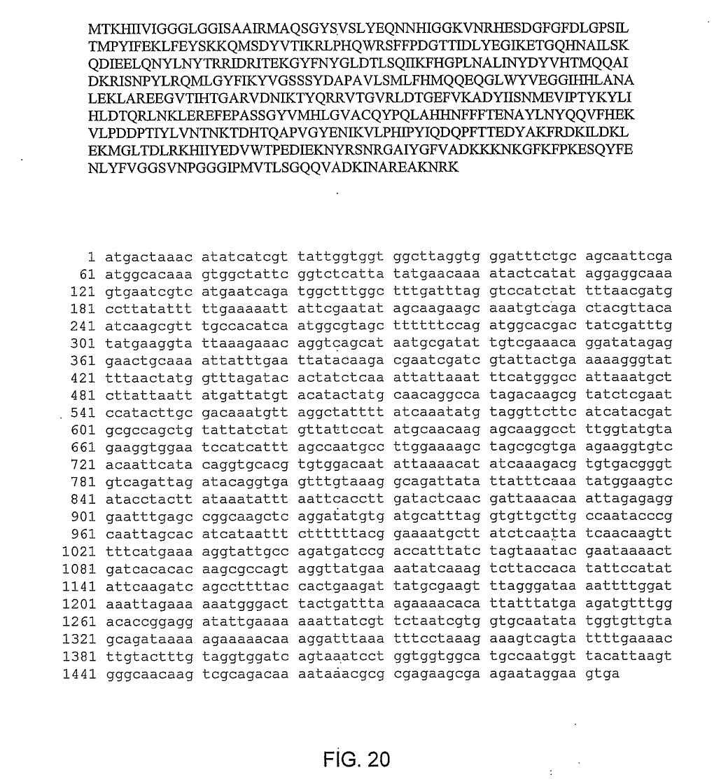

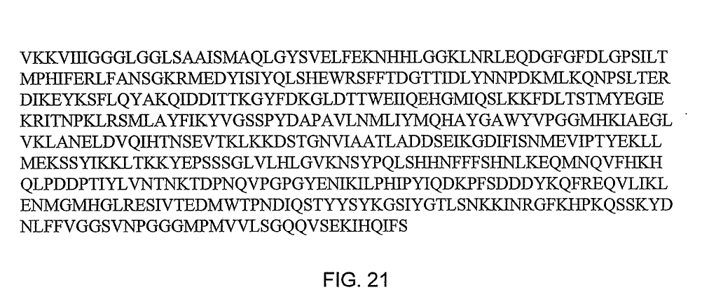

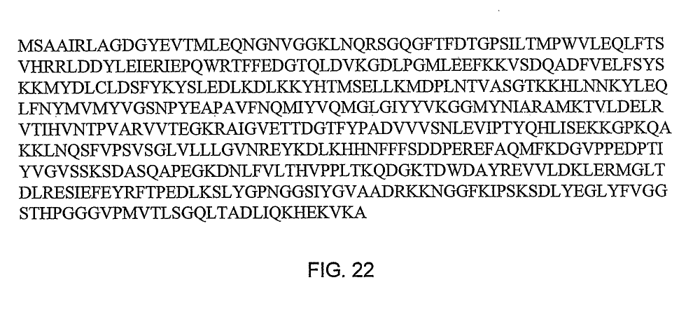

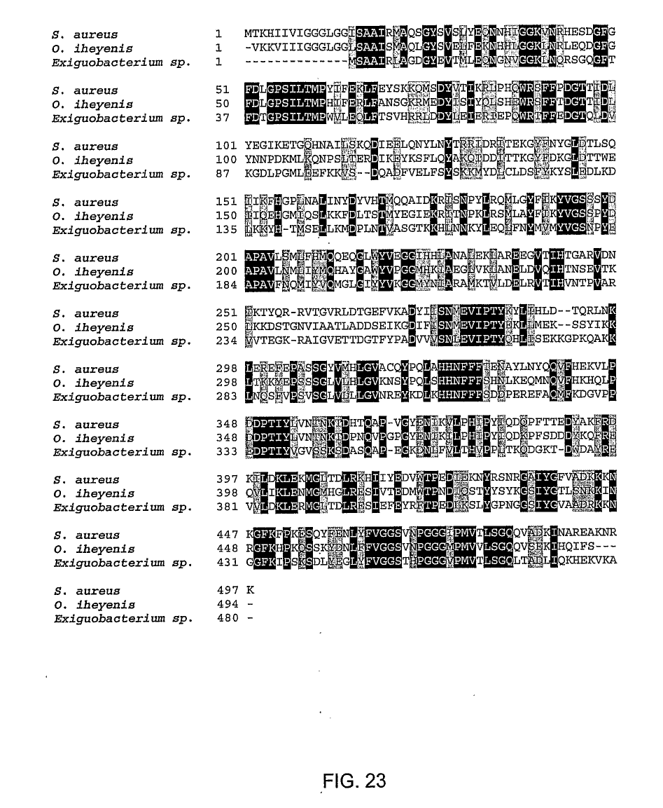

Suitable genes encoding carotenoid oxygenases include CrtOx from S. aureus (GenBank Accession No. CAA66626.1); CrtOx from Oceanobacillus iheyensis (GenBank Accession Nos. NC_{—}004193); and ORF6 from Methylobacterium extorquens (TIGR Accession No. RMQ04999, contig1482_{—}20719_{—}22191). See, also, FIG. 20. The amino acid sequences of the carotenoid oxygenases from S. aureus, O. iheyensis, and Exiguobacterium sp. 255-15 can be found in GenBank under Accession Nos. NP_{—}373088, NP_{—}693380, and ZP_{—}00183789, respectively. See, also FIGS. 20-23.

Genes encoding CrtGT and CrtAT have been identified from Staphylococcus aureus and Oceanobacillus iheyensis. For example, the nucleic acid and amino acid sequences of CrtGT and CrtAT from Staphylococcus aureus and Oceanobacillus iheyensis can be found in GenBank (Accession Nos. NC_{—}002758; NP_{—}373087; NP_{—}373089; NC_{—}004193; NP_{—}693379; and NP_{—}693378.

In some cases, mutant carotenoid oxygenases can be made and used. For example, in vitro-evolved CrtOx mutants can be made as described herein and can be used to engineer microorganisms capable of producing more 2,4,2′,4′-tetradehydrolycopendial than lycopene (e.g., the ratio of 2,4,2′,4′-tetradehydrolycopendial to lycopene can be greater than 3:1, 4:1, 5:1, 6:1, 7:1, 8:1, 9:1, 10:1; 11:1, 12:1, 13:1, 14:1, 15:1, 16:1, 17:1, 18:1, 19:1, or 20:1). Such carotenoid oxygenases can be crtOx(SA)ₘᵤₜ₁, crtOx(SA)ₘᵤₜ₂, or crtOx(SA)ₘᵤₜ₃. It will be appreciated that for any of the methods and material provided herein, a mutant carotenoid oxygenase can be used in combination with a wild-type carotenoid oxygenase or in place of a wild-type carotenoid oxygenase.

Nucleic acids encoding FPP synthases have been identified from E. coli (IspA), Bacillus subtilis, Arabidopsis thaliana, Neurospora crassa, Gallus gallus, and Homo sapiens. The nucleic acid sequence encoding IspA is available in GenBank under Accession No. AAC73524. A number of genes encoding the enzymes for central carotenoid biosynthetic routes have been cloned and genes from different species have been shown to function cooperatively when combined.

CrtM and CrtN also can be used in combination with lycopene cyclase (CrtY) to produce diapotorulene, a cyclic derivative of diaponeurosporene. CrtY catalyzes the introduction of β-rings into either end of lycopene to synthesize β,β-carotene, which can be further modified. Genes encoding CrtY have been identified in a variety of species, including Pantoea species (formerly Erwinia). For example, crtY can be used from P. ananatis (GenBank Accession No. D90087). Alternatively, a modified crtY such as crtY2 can be used. See, for example, U.S. Patent Application 20020051998 and Schmidt-Dannert et al. (2000) Nat. Biotech. 18:75-753. CrtY2 is a variant that cyclizes didehydrolycopene, the precursor of tetradehydrolycopene, to produce the red carotenoid torulene. Farnesyl diphosphate synthase (e.g., IspA from E. coli) can be used to increase production of diapotorulene relative to diaponeurosporene.

Acyclic C35 ketocarotenoids can be produced using CrtN and CrtM in combination with spheroidene monooxygenase (CrtA), which catalyzes the oxygenation of spheroidene or hydroxysphroidene at C2. Genes encoding CrtA are available from a variety of microorganisms, including Rhodobacter (e.g., R. capsulatus, GenBank Accession No. Z11165). Microorganisms expressing such nucleic acids are more yellow in color than microorganisms expressing only CrtN and CrtM.

In other embodiments, acyclic carotenoids can be produced in microorganisms using a nucleic acid encoding geranyl geranyl diphosphate (GGDP) synthase (CrtE), phytoene synthase (CrtB), and phytoene desaturase (CrtI) in combination with a nucleic acid encoding one or more additional carotenoid enzymes. Such nucleic acids can be part of the same construct or on different constructs. Genes encoding CrtE, CrtB, and CrtI have been identified from a variety of species, including, for example, Pantoea (see GenBank Accession No. D90087). A modified CrtI such as CrtI₁₄, a six-step phytoene desaturase capable of synthesizing the fully conjugated 3,4,3′,4′-tetradehydrolycopene in E. coli, also can be used. See, for example, U.S. Patent Application 20020051998 and Schmidt-Dannert et al. (2000) supra. Microorganisms expressing crtE, crtB, and crtI accumulate lycopene, while microorganisms expressing crtE, crtB, and crtI₁₄ accumulate tetradehydrolycopene. Alternatively, tetradehydrolycopene can be produced in microorganisms using a five step desaturase from Neurospora crassa (GenBank Accession No. M57465) in place of crtI₁₄. Acyclic xanthophylls such as ζ-carotene-2-one, neurosporene-2-one, and lycopene-2-one can be produced by introducing a nucleic acid encoding spheroidene monooxygenase (CrtA) such as the CrtA from Rhodobacter into a crtE, crtB, and crtI-containing microorganism. Phillipsiaxanthin, a deep purple carotenoid, can be produced by introducing a nucleic acid encoding CrtA into a microorganism containing crtE, crtB, and crtI₁₄. As indicated above, the gene encoding the five-step desaturase from N. crassa can be used in place of crtI₁₄.

### Production of Torulene Derivatives

To produce torulene derivatives such as ketotorulene, an exogenous nucleic acid encoding a β-carotene oxygenase (CrtO, also known as β-carotene ketolase) such as the CrtO from Synechocystis sp. PCC 6803 (GenBank Accession No. D64004) can be introduced into a microorganism containing crtE, crtB, crtI₁₄, and crtY2. Aromatic torulene (didehydro-βφ-carotene) can be produced by introducing an exogenous nucleic acid encoding β-carotene desaturase (CrtU) into a microorganism containing crtE, crtB, crtI₁₄, and crtY2. Suitable genes encoding CrtU have been identified from Streptomyces griseus, Mycobacterium aurum, or Brevibacterium linens (GenBank Accession No. AF139916). Microorganisms containing the five-step desaturase from N. crassa also make torulene and can be used in place of the modified enzymes.

Hydroxytorulene can be produced in a microorganism by introducing an exogenous nucleic acid encoding β-carotene hydroxylase (CrtZ) such as the CrtZ from Pantoea (GenBank Accession No. D90087) into a microorganism containing crtE, crtB, crtI₁₄, and crtY. An exogenous nucleic acid encoding zeaxanthin glucosylase (CrtX) can be introduced into a microorganism containing crtE, crtB, crtI₁₄, crtY, and crtZ to produce torulene glucoside.

### Producing Carotenoids

The microorganisms described herein can be used to produce carotenoids (e.g., diapolycopene dialdehyde, diapolycopene dicarboxylic acid, diapotorulene, acyclic C35 ketocarotenoids, tetradehydrolycopene, acyclic xanthophylls, ketotorulene, or hydroxytorulene). For example, as discussed above, one or more exogenous nucleic acids can be introduced into a microorganism and cultured under conditions optimal for carotenoid production.

In addition, substantially pure polypeptides having enzymatic activity can be used alone or in combination with microorganisms to produce carotenoids. The term “substantially pure” as used herein with reference to a polypeptide means the polypeptide is substantially free of other polypeptides, lipids, carbohydrates, and nucleic acid with which it is associated in nature. A substantially pure polypeptide can be at least about 60, 65, 70, 75, 80, 85, 90, 95, or 99 percent pure. Typically, a substantially pure polypeptide will yield a single major band on a polyacrylamide gel.

In one embodiment, the invention provides a substantially pure polypeptide having one or more of the following activities: a synthase (e.g., dehydrosqualene synthase, EC 2.5.1.-; diapophytoene synthase; phytoene synthase, EC 2.5.1.32; or geranyl geranyl diphosphate synthase, EC 2.5.1.29), desaturase (e.g., phytoene desaturase, EC 1.14.99.30), or oxygenase (e.g., spheroidene monooxygenase) activity. In another embodiment, the invention provides a composition that contains two or more (e.g., three, four, five, six, seven, eight, nine, ten, or more) substantially pure polypeptide preparations. For example, a composition can contain a substantially pure polypeptide preparation of the diapophytoene synthase polypeptide from S. aureus and a substantially pure polypeptide preparation of the dehydrosqualene synthase polypeptide from S. aureus. Such compositions can be in the form of a container. For example, two or more substantially pure polypeptide preparations can be located within a column. In some embodiments, the polypeptides can be immobilized on a substrate such as a resin.

Any method can be used to obtain a substantially pure polypeptide. For example, common polypeptide purification techniques such as affinity chromatography and HPLC as well as polypeptide synthesis techniques can be used. In addition, any material can be used as a source to obtain a substantially pure polypeptide. For example, tissue from wild-type or transgenic animals can be used as a source material. In addition, tissue culture cells engineered to over-express a particular polypeptide of interest can be used to obtain a substantially pure polypeptide. Further, a polypeptide within the scope of the invention can be “engineered” to contain an amino acid sequence that allows the polypeptide to be captured onto an affinity matrix. For example, a tag such as c-myc, hemagglutinin, polyhistidine, or Flag™ tag (Kodak) can be used to aid polypeptide purification. Such tags can be inserted anywhere within the polypeptide including at either the carboxyl or amino termini. Other fusions that can be used include enzymes such as alkaline phosphatase that can aid in the detection of the polypeptide.

For example, a preparation containing substantially pure polypeptides having dehydrosqualene synthase, diapophytoene synthase, and carotenoid oxidoreductase activity can be used to catalyze the formation C30 purple carotenoids such as diapolycopene dialdehyde and diapolycopene dicarboxylic acid. Further, cell-free extracts containing a polypeptide having enzymatic activity can be used alone or in combination with substantially pure polypeptides and/or cells to produce carotenoids. Any method can be used to produce a cell-free extract. For example, osmotic shock, sonication, and/or a repeated freeze-thaw cycle followed by filtration and/or centrifugation can be used to produce a cell-free extract from intact cells. It is noted that a microorganism, substantially pure polypeptide, and/or cell-free extract can be used to produce any carotenoid that is, in turn, treated chemically to produce another compound. Likewise, a chemical process can be used to produce a particular compound that is, in turn, converted into a carotenoid using a cell, substantially pure polypeptide, and/or cell-free extract described herein.

Typically, carotenoids are produced by providing a microorganism and culturing the provided microorganism with a suitable culture medium. In general, the culture media and/or culture conditions can be such that the microorganisms grow to an adequate density and produce carotenoids efficiently. For large-scale production processes, any method can be used such as those described elsewhere (Manual of Industrial Microbiology and Biotechnology, 2^{nd} Edition, Editors: A. L. Demain and S. E. Davies, ASM Press; and Principles of Fermentation Technology, P. F. Stanbury and A. Whitaker, Pergamon). Briefly, a large tank (e.g., a 100 gallon, 200 gallon, 500 gallon, or more tank) containing appropriate culture medium with, for example, a glucose carbon source is inoculated with a particular microorganism. After inoculation, the microorganisms are incubated to allow biomass to be produced. Once a desired biomass is reached, the broth containing the microorganisms can be transferred to a second tank. This second tank can be any size. For example, the second tank can be larger, smaller, or the same size as the first tank. Typically, the second tank is larger than the first such that additional culture medium can be added to the broth from the first tank. In addition, the culture medium within this second tank call be the same as, or different from, that used in the first tank. For example, the first tank can contain medium with glucose, while the second tank can contain medium with glycerol.

Once transferred, the microorganisms can be incubated to allow for the production of a carotenoid. Once produced, any method can be used to isolate the carotenoids. For example, common separation techniques can be used to remove the biomass from the broth, and common isolation procedures (e.g., extraction, distillation, and ion-exchange procedures) can be used to obtain the carotenoid from the biomass.

Typically, a microorganism of the invention produces the carotenoids of interest at a concentration of at least about 1 mg per L (e.g., at least about 2.5 mg/L, 5 mg/L, 10 mg/L, 20 mg/L, 25 mg/L, 50 mg/L, 75 mg/L, 80 mg/L, 90 mg/L, 100 mg/L, or 120 mg/L). When determining the yield of a carotenoid for a particular microorganism, any method can be used. See, e.g., Applied Environmental Microbiology 59(12):4261-4265 (1993).

### Compositions

Compositions of the invention can be purified carotenoid compounds (e.g., neurosporene-2-one, ζ-carotene-2-one, lycopene-2-one, phillipsiaxanthin, hydroxytorulene, torulene glucoside, ketotorulene, didehydro-β,φ-carotene, diapotorulene, diapolycopene, 4,4′-diapo-ζ-carotene-al, a C35 carotenoid, 4,4′-diapo-lycopene-dial, 4,4′-diapo-ζ-carotene-dial, 4,4′-diapo-lycopene-al-oic acid, or a water soluble carotenoid such as norbixin), or combinations of carotenoid compounds, crude extracts containing one or more carotenoids, or the dried biomass. Crude extracts can be prepared from microorganisms using standard techniques, including, for example, extraction with an organic solvent such as methanol or acetone. Chromatographic techniques such as high-performance liquid chromatography (HPLC) or thin-layer chromatography (TLC) can be used to further purify the crude extracts. In other embodiments, the microorganisms producing the carotenoids (i.e., the biomass) are collected and dried. Compositions can be used in pharmaceutical compositions, nutraceuticals, cosmetics, food or feed compositions, or as antioxidant supplements.

The invention will be further described in the following examples, which do not limit the scope of the invention described in the claims.

### EXAMPLES

### Example 1

### Methods and Materials

### Cloning and Culture Growth

Genes encoding dehydrosqualene synthase (crtM) from Staphylococcus aureus (crtM(SA); ATCC 35556D), diapophytoene synthase (crtN) from Staphylococcus aureus (crtN(SA); ATCC 35556D), spheroidene monooxygenase (crtA) from Rhodobacter capsulatus (crtA(RC); DSMZ 1710), β-carotene oxygenase (crtO) from Synechocystis sp. (crtO(SS); ATCC 27184), β-carotene desaturase (crtU) Brevibacterium linens (crtU(BL); DSMZ 20426), β-carotene hydroxylase (crtZ) from Erwinia uredovora (crtZ(EU); Pantoea ananatis DSMZ 30080), and zeaxanthin glucosylase (crtX) from Erwinia uredovora (crtX(EU); Pantoea ananatis DSMZ 30080) were amplified from genomic DNA using a 5′ primer containing at its 5′ end a XbaI or EcoRI site followed by an optimized Shine-Dalgarno sequence (underlined) and a start codon (bold) (5′-AGGAGGATTACAAAATG-3′, SEQ ID NO:1) and a 3′ primer containing at its 5′ end a EcoRI or NcoI site (Table 1A). PCR products were then digested with restriction enzymes and cloned into the corresponding sites of plasmid pUCmod (Schmidt-Dannert et al., Nat. Biotechnol., 18:750-753 (2000)) to facilitate constitutive expression from a modified lac-promoter.

TABLE 1
Genes (A) and plasmids (B).

| (A) Gene | Enzyme | Typical reaction catalyzed | Accession no. or Reference |
|---|---|---|---|
| crtM | Dehydrosqualene | Head-to-head condensation | X73889 |
| | synthase | of 2 FDP | |
| crtN | Diapophytoene | Introduction of 3 desaturations | X73889 |
| | synthase | in dehydrosqualene | |
| crtE | GGDP synthase | Head-to-head condensation | D90087 |
| | | of IDP + FDP | |
| crtB | Phytoene synthase | Head-to-head condensation | D90087 |
| | | of 2 GGDP | |
| crtI | Phytoene desaturase | Introduction of 4 desaturations | D90087 |
| | | in phytoene | |
| crtI₁₄ | In vitro evolved | Introduction of 6 desaturations | Schmidt-Dannert et al. |
| | phytoene desaturase | in phytoene | (2000), supra |
| crtY | Lycopene cyclase | Cyclization of ψ-end groups in | D90087 |
| | | lycopene to form β-rings | |
| crtY2 | In vitro evolved | Cyclization of ψ-end group in | Schmidt-Dannert et al. |
| | lycopene cyclase | didehydrolycopene to form | (2000), supra |
| | | β-ring | |
| crtA | Spheroidene | Oxygenation at C2 of spheroidene | Z11165 |
| | monooxygenase | or hydroxysphroidene | |
| crtO | β-carotene | Oxygenation at C4, C4′ of | D64004 |
| | oxygenase | β-carotene | |
| crtU | β-carotene | Desaturation/methyltransfer | AF139916 |
| | desaturase | of β-rings in β-carotene | |
| crtZ | β-carotene | Hydroxylation of C3, C3′ of | D90087 |
| | hydroxylase | β-carotene | |
| crtX | Zeaxanthin | Glycosylation of C3, C3′ of | D90087 |
| | glucosylase | zeaxanthin | |

| (B) Plasmid | Properties | Reference |
|---|---|---|
| pUCmod | Constitutive expression vector modified | Schmidt-Dannert et al. |
| | from pUC19, Ap | (2000), supra |
| pACmod | Cloning vector modified from | Schmidt-Dannert et al. |
| | pACYC184, Cm | (2000), supra |
| pUC-crtM(SA) | pUCmod constitutively expressing crtM | Herein |
| | (S. aureus; SA) | |
| pUC-crtN(SA) | pUCmod constitutively expressing crtN | Herein |
| | (S. aureus; SA) | |
| pUC-crtY(EU) | pUCmod constitutively expressing crtY | Schmidt-Dannert et al. |
| | (Erwinia Uredovora; EU) | (2000), supra |
| pUC-crtY2(EU/EH) | pUCmod constitutively expressing crtY2 | Schmidt-Dannert et al. |
| | (chimeric; Erwinia herbicola; EH) | (2000), supra |
| pUC-crtA(RC) | pUCmod constitutively expressing crtA | Herein |
| | (Rhodobacter capsulatus; RC) | |
| pUC-crtO(SS) | pUCmod constitutively expressing crtO | Herein |
| | (Synechocystis sp. PCC6803; SS) | |
| pUC-crtU(BL) | pUCmod constitutively expressing crtU | Herein |
| | (Brevibacterium linens; BL) | |
| pUC-crtZ(EU) | pUCmod constitutively expressing crtZ | Herein |
| | (Erwinia Uredovora; EU) | |
| pUC-crtX(EU) | pUCmod constitutively expressing crtX | Herein |
| | (Erwinia Uredovora; EU) | |
| pAC-crtM(SA)-crtN(SA) | pACmod constitutively expressing crtM | Herein |
| | and crtN to produce diaponeurosporene | |
| pAC-crtE(EU)-crtB(EU)-crtI(EU) | pACmod constitutively expressing crtE, | Schmidt-Dannert et al. |
| | crtB and crtI to produce lycopene | (2000), supra |
| pAC-crtE(EU)-crtB(EU)-crtI₁₄ | pACmod constitutively expressing crtE, | Schmidt-Dannert et al. |
| | crtB and mutant crtI₁₄ to produce | (2000), supra |
| | tetradehydrolycopene | |
| pAC-crtE(EU)-crtB(EU)-crtI₁₄- | pACmod constitutively expressing crtE, | Herein |
| crtY(EU) | crtB, mutant crtI₁₄ and crtY to | |
| | produce β-carotene | |
| pAC-crtE(EU)-crtB(EU)-crtI₁₄- | pACmod constitutively expressing crtE, | Herein |
| crtY2(EU/EH) | crtB, mutant crtI₁₄ and mutant crtY2 | |
| | to produce torulene | |
| pAC-crtE(EU)-crtB(EU)-crtI₁₄- | pACmod constitutively expressing crtE, | Herein |
| crtY(EU)-crtZ(EU) | crtB, mutant crtI₁₄, crtY and crtZ | |
| | to produce zeaxanthin | |
| pAC-crtE(EU)-crtB(EU)-crtI₁₄- | pACmod constitutively expressing crtE, | Herein |
| crtY2(EU/EH)-crtZ(EU) | crtB, mutant crtI₁₄, mutant crtY2 and | |
| | crtZ to produce monohydroxytorulene | |

For C30 carotenoid pathway assembly, crtM(SA) and crtN(SA) were subcloned from pUCmod into the SalI (crtM) or a BamHI (crtN) site of pACmod (see Schmidt-Dannert et al. (2000) supra) by amplification of the genes together with the modified constitutive lac-promoter, using primers that introduce the corresponding restriction enzyme sites at both ends, to give pAC-crtM(SA)-crtN(SA), where crtM(SA) and crtN(SA) have the same orientation as the disrupted tetracycline resistance gene. Likewise, for assembly of the β-carotene and torulene pathways, genes encoding wild-type (crtY(EU)) or mutant lycopene cyclase (crtY2) were subcloned from pUCmod into the SalI site of pAC-crtE(EU)-crtB(EU)-crtI₁₄ (see Schmidt-Dannert et al. (2000) supra) to give pAC-crtE(EU)-crtB(EU)-crtI₁₄-crtY(EU) and pAC-crtE(EU)-crtB(EU)-crtI₁₄-crtY2(EU/EH), respectively (crtY/Y2 have the same orientation as crtE and crtI₁₄). To assemble the glucosylation pathways, crtZ(EU) was subcloned similarly into the PpmUI site of pAC-crtE(EU)-crtB(EU)-crtI₁₄-crtY(EU) and pAC-crtE(EU)-crtB(EU)-crtI₁₄-crtY2(EU/EH) to produce pAC-crtE(EU)-crtB(EU)-crtI₁₄-crtY(EU)-crtZ(EU) and pAC-crtE(EU)-crtB(EU)-crtI₁₄-crtY2(EU/EH)-crtZ(EU), respectively (crtZ(EU) has the same orientation as crtY/Y2). These plasmids and the carotenoids biosynthetic pathways expressed are described in Table 1B.

For carotenoid production, recombinant E. coli JM109 were cultivated for 48 hours in the dark at 28° C. in Luria-Bertani (LB) medium (200 mL medium in a 500 mL flask or 1 l medium in a 3 L flask) supplemented with the appropriate selective antibiotics chloramphenicol (50 μg/mL) and/or carbenicillin (100 μg/mL).

### Isolation of Carotenoids

Wet cells from a 200 mL (˜500 mg) or 4 L culture (˜10 g) were repeatedly extracted at 4° C. with a total volume of 30 mL or 400 mL methanol or acetone until all visible pigments were extracted. After centrifugation (4° C., 6000 rpm), the colored supernatants were pooled and combined supernatants were centrifuged again, filtrated (nylon membrane 0.2 μm, Whatman) to remove fine particles, evaporated in a vacuum to dryness and finally resuspended with 30-50 mL acetone. The acetone extract was kept at −80° C. for one day to form a white precipitate and filtrated with 0.2 μm nylon membrane to remove the precipitate. The resulting pigment extracts were re-extracted with an equal volume of ethyl acetate or hexane after addition of ½ volume of saltwater (15% NaCl). The organic phase that contained carotenoids was collected and washed with water. The collected organic phase was completely evaporated in a vacuum to dryness at room temperature, resuspended with 0.5-1 mL hexane, applied to silica gel chromatography (25×120 cm) and eluted stepwise with increasing amount of acetone in hexane (0% acetone to 30% acetone in hexane basis). The color fractions were then dried under nitrogen gas or in a vacuum and dissolved in 1-2 mL hexane. A 1-3 μL aliquot of the fractions and the crude extracts were subjected to high-performance TLC separation for initial analysis of the crude extract and the color fractions composition on Whatman silica gel 60 Å plates (4.5 μm particle size, 200 μm thickness) using the following solvent systems: i) acetone:hexane (40:60) for acyclic C30 and C40 xanthophylls, ii) hexane:chloroform:acetone (85:15:20) for diapocarotenoids and cyclic xanthophylls, iii) hexane:chloroform (85:15) for cyclic aromatic carotenoids, iv) hexane:chloroform (100:5) for cyclic C40 carotenoids and v) hexane:acetone (80:20) for hydroxylated cyclic C40 carotenoids and vi) chloroform:methanol (80:20) for glucosylated cyclic C40 carotenoids. For the further purification of carotenoids, a preparative TLC and HPLC were used. The preparative TLC was performed under the same conditions as the above and carotenoids were eluted with acetone or methanol. The preparative HPLC, if needed, was carried out with a semi-preparative Zorbax SB-C18 column (9.6×250 mm, 5 μm; Agilent Technologies, Palo Alto, Calif.), and eluted under isocratic conditions with two solvent systems [A; 90% acetonitrile and 10% methanol and B; 90% (acetonitrile:water, 100:15) and 10% methanol] at a flow rate of 1.5 ml min⁻¹, which were optimized based on peak resolution, using an Agilent 1100 HPLC system equipped with an photodiode array detector.

### Analysis of Carotenoids

For the analysis of carotenoids, 10-20 μL of the crude extract and the collected color fractions were applied to a Zorbax SB-C18 column (4.6×250 mm, 5 μm; Agilent Technologies, Palo Alto, Calif.), and typically eluted under isocratic conditions with a solvent system containing 90% (acetonitrile: H₂O, 99:1) and 10% (methanol: tetrahydrofurane, 8:2) at a flow rate of 1 ml min⁻¹ using an Agilent 1100 HPLC system equipped with an photodiode array detector. Gradient conditions with solvent A (acetonitrile: H₂O, 85:15) and solvent B (methanol: tetrahydrofurane, 8:2) were used for the elution of acyclic C₄₀ xanthophylls (0-30 min, A:B 95:5; 30-60 min, A:B 88:12; 60-90 min, A:B 1:1; 90-120 min, A:B 1:9). For structural elucidation, carotenoids were identified by a combination of HPLC retention times, absorption spectra and mass fragmentation spectra. See, Schwieter, et al., (1966) Helv. Chim. Acta 49, 992-996; Enzell et al., (1968) Acta Chem. Scand. 22, 1054-1055; and Enzell et al., Acta Chem. Scand. 23, 727-750. Authentic standards for comparison were isolated from recombinant E. coli containing plasmids for lycopene, tetradehydrolycopene, torulene and β,β-carotene biosynthesis. Mass fragmentation spectra were monitored in a mass range of m/z 200-800 or 1000 on a LCQ mass spectrophotometer equipped with an electron spray ionization (ESI) or atmosphere pressure chemical ionization (APCI) interface (Thermo Finnigan, USA). Parent molecular ions were further fiagmented by MS/MS analysis using an APCI interface at optimal collision-induced dissociation energy (28-30%).

### Example 2

### Co-Expression of Dehydrosqualene Synthase CrtM and Desaturase CrtN Produces the Fully Conjugated C30 Carotenoid Diapolycopene

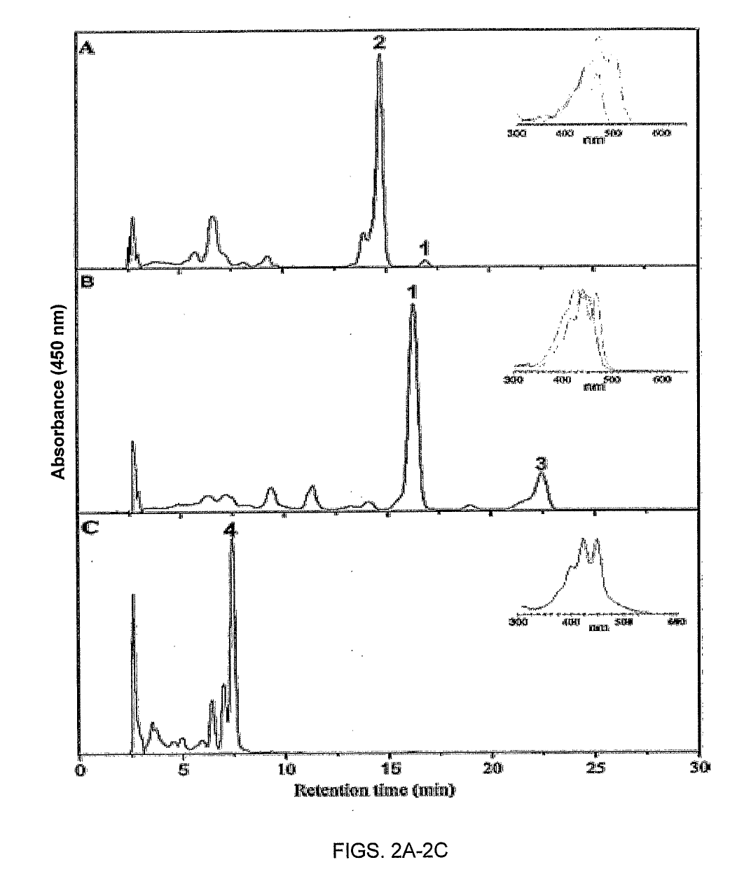

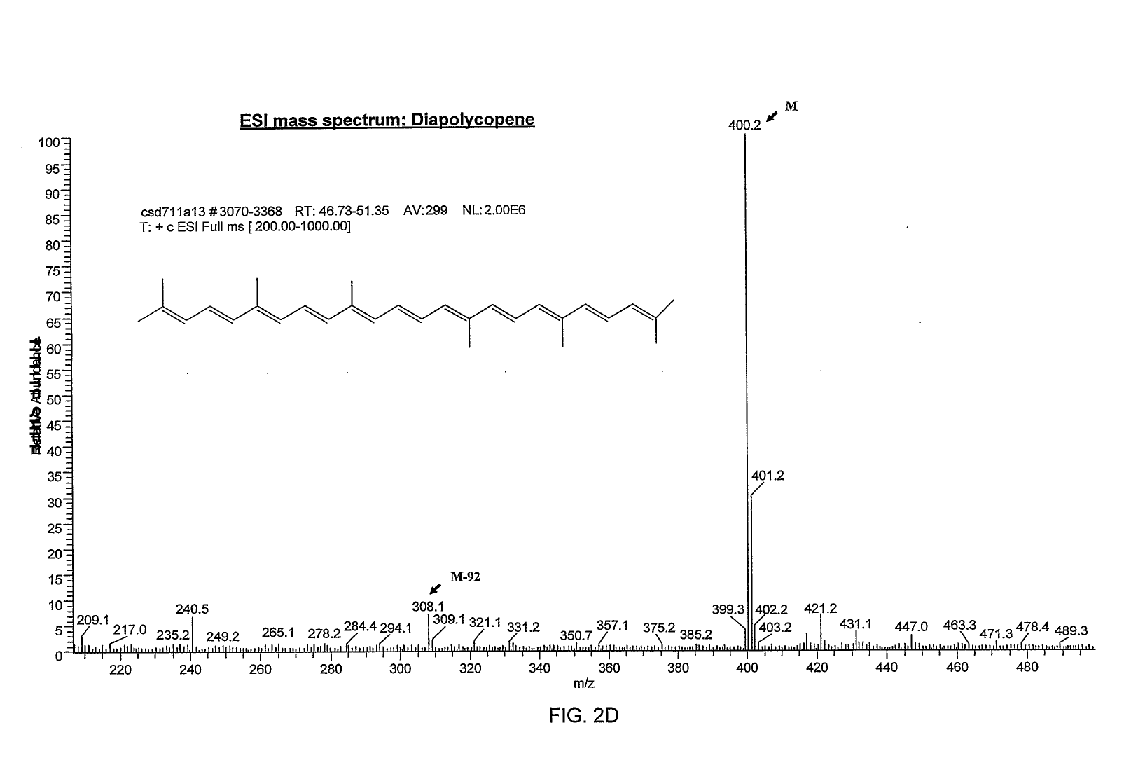

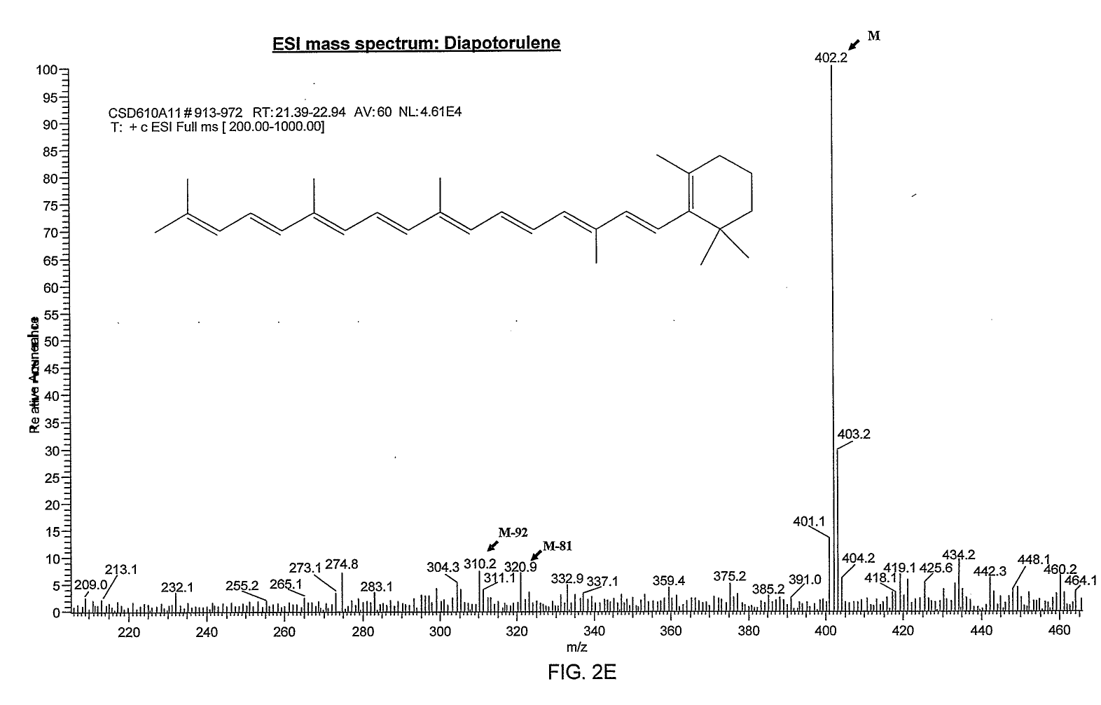

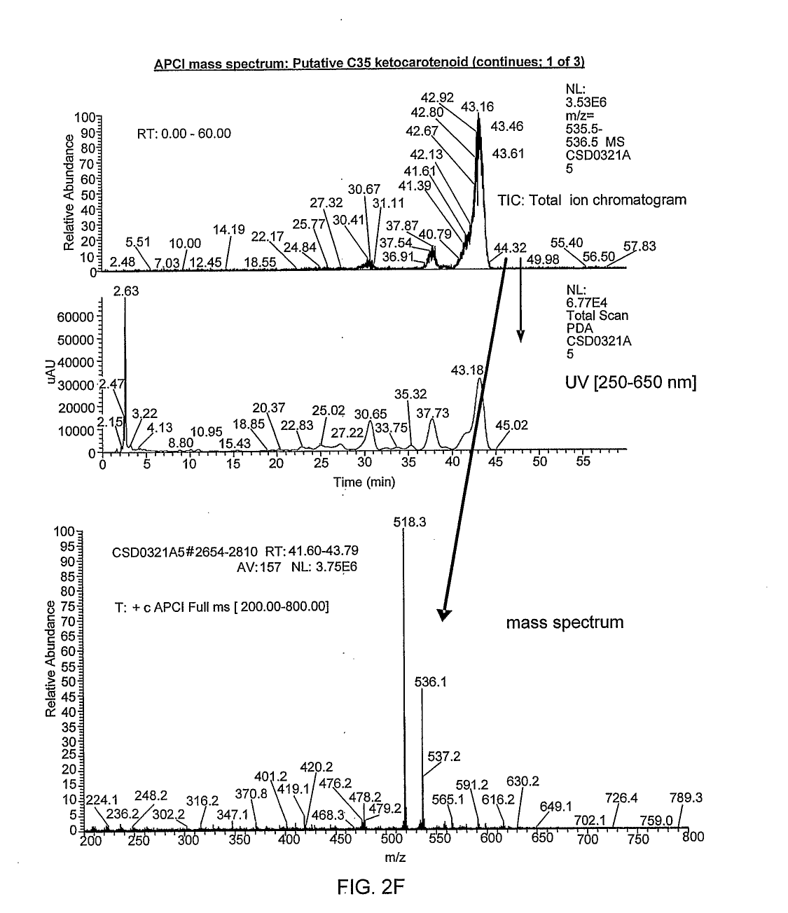

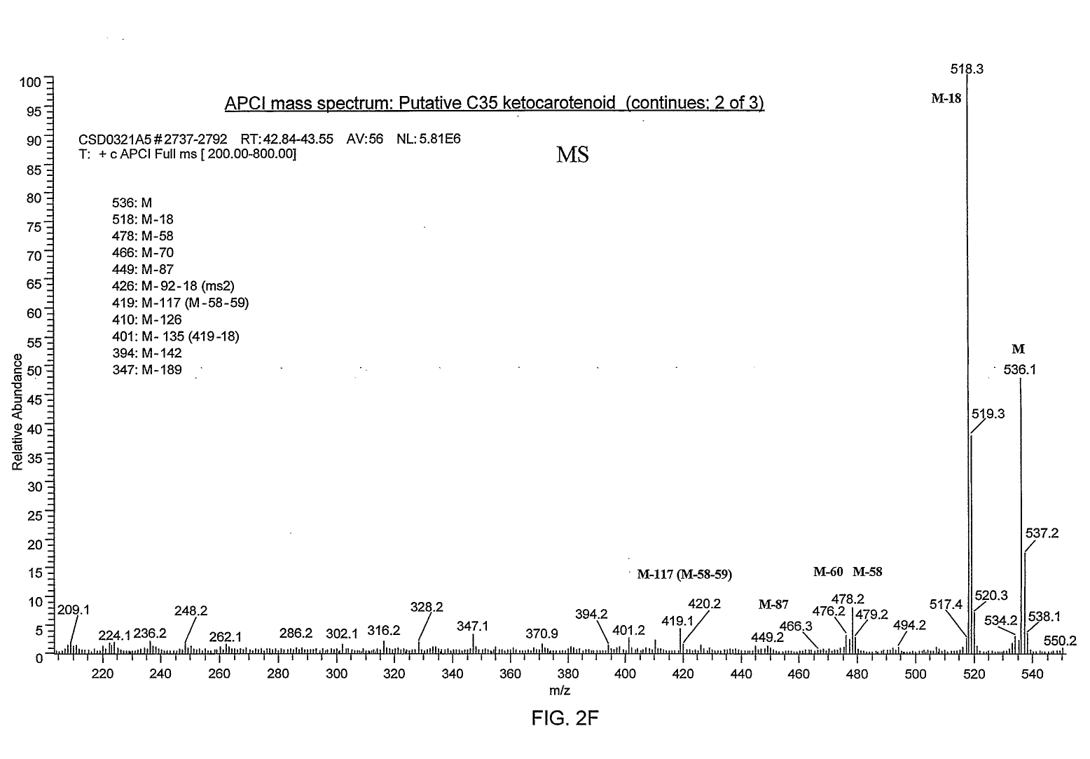

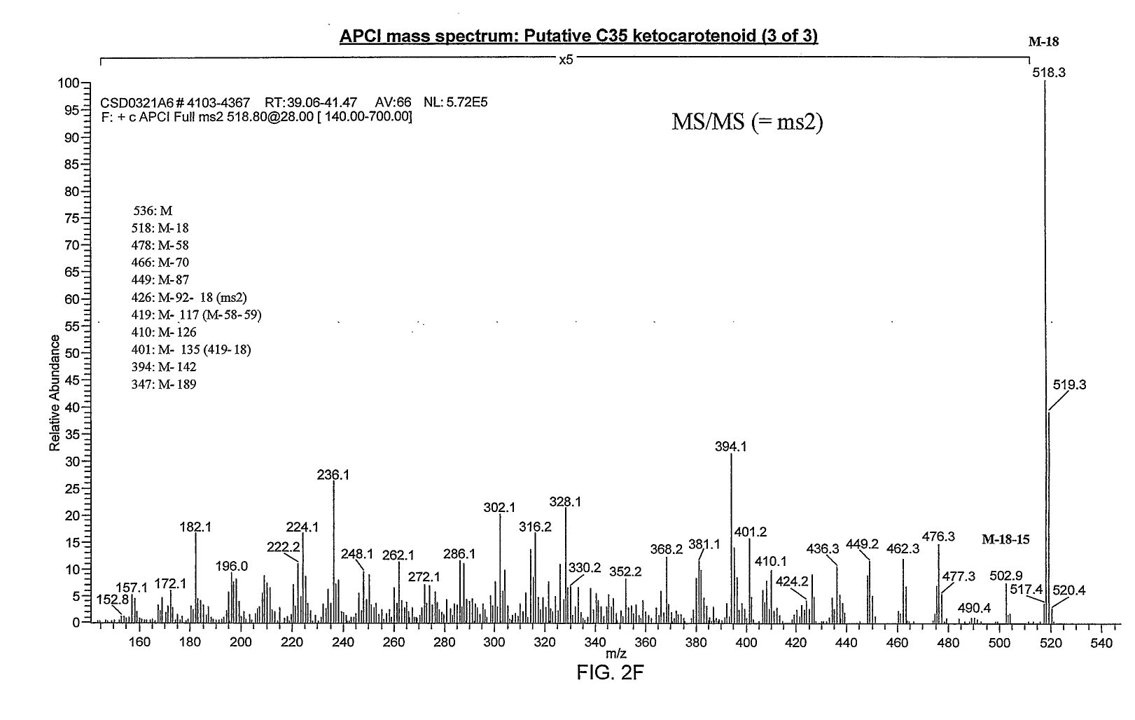

To extend the isoprenoid pathway in E. coli for synthesis of C₃₀ carotenoids, two expression cassettes comprising a constitutive lac-promoter upstream of either crtM(SA) or crtN(SA) were assembled to yield pAC-crtM(SA)-crtN(SA). E. coli cells transformed with pAC-crtM(SA)-crtN(SA) developed a deep yellow-orange color suggesting the production of diapocarotenoids. Analysis of the cell extracts by HPLC-mass spectrometry showed that, in this system, CrtN efficiently introduced four double bonds into dehydrosqualene to predominantly (90%) synthesize the fully conjugated 4,4′-diapolycopene in recombinant E. coli (FIG. 2A). The ESI mass spectrum of diapolycopene is shown in FIG. 2D. This is in contrast to earlier reports where CrtN was shown to catalyze efficiently the three step desaturation of dehydrosqualene leading to the formation of 4,4′-diaponeurosporene in recombinant E. coli (see Wieland, et al., (1994). J. Bacteriol. 176, 7719-7726). However, Arnold et al. reported the accumulation of 30% diapolycopene in recombinant E. coli cells constructed for directed evolution studies aimed at evolving CrtM for function in a C₄₀ pathway (see Umeno et al. (2002). J. Bacteriol. 184, 6690-6699). Unexpectedly, it was observed that E. coli cells harboring pAC-crtN(SA)-crtM(SA) also accumulated significant amounts of polar carotenoids. Molecular masses and absorption spectra showed them to be various diapolycopene and diaponeurosporene derivatives carrying methoxy and/or hydroxy-functional groups at one or both of their ends. Acyclic end groups of bacterial C₃₀ diapocarotenoids are frequently oxidized to hydroxy, aldehyde or carboxy-groups, which can be further acylated and/or glucosylated. The diapocarotenoid end-groups are prone to oxidation by free peroxyl-radicals (especially hydroperoxyl radicals) formed in lipid membranes during oxygen stress. The observed methoxy-groups may have formed from hydroperoxyl-groups in the presence of methanol present during isolation and analysis. Significant modification of C₄₀ carotenoids was not observed, indicating that the orientation of the C₃₀ carotenoids in the lipid membrane of E. coli may be different and thus increasing its reactivity with reactive oxygen species like peroxyl-radicals.

### Example 3

### Lycopene Cyclase CrtY Cyclizes the C30 Carotenoid Diaponeurosporene

Cyclization of C₃₀ diapocarotenoids, which is a common modification of C₄₀ carotenoids, is so far unknown. Because lycopene cyclase CrtY acts on ψ-end groups, which are the same in acyclic C₄₀ carotenoids (like e.g. lycopene) and C₃₀ carotenoids (like diaponeurosporene or diapo-ζ-carotene), it was reasoned that expression of crtY on pUC-crtY(EU) together with the genes for diapolycopene biosynthesis on pAC-crtM(SA)-crtN(SA), would produce novel unnatural cyclic diapocarotenoids in E. coli. Indeed, a novel cyclic carotenoid along with diaponeurosporene was detected in cell extracts of such co-transformed recombinant E. coli cells (FIG. 2B). Absorption and mass spectrum confirmed it to be diapotorulene, the cyclic derivative of diaponeurosporene. The ESI mass spectrum of diapotorulene is shown in FIG. 2E. Other possible monocyclic and dicyclic diapocarotenoids derived from diapo-ζ-carotene were not detected. As farnesyl diphosphate (FDP) is the precursor of the C30 biosynthetic pathway, the native E. coli FDP synthase (IspA(EC)) was over-expressed in order to increase the precursor pool and alter production levels. Expression of the resulting construct (pAC-ispA(EC)-crtM(SA)-crtN(SA)) in E. coli increased the diapotorulene to diaponeurosporene ratio 3-5-fold.

### Example 4

### Spheroidene Monooxygenase CrtA Oxygenizes Acyclic Intermediates of the Diapo-Phytoene (C30) Desaturation Pathway

Although many bacteria produce a large number of different acyclic xanthophylls (oxygenated carotenoids), only four genes encoding a hydratase (crtC), desaturase (crtD), methyl transferase (crtF), and a monooxygenase (crtA) have been cloned from Rhodobacter strains (see Armstrong et al (1989) Mol. Gen. Genet. 216:254-268). To obtain acyclic carotenoids with expanded chromophores, CrtA was chosen as a possible enzyme for the introduction of keto-groups into diapolycopene. In purple bacteria under aerobic conditions, CrtA catalyzes the asymmetrical introduction of one keto-group at C2 as the terminal reaction of a sequence involving first hydroxylation at C1, C1′ (CrtC) of neurosporene or lycopene, followed by desaturation at C3,C4 (C3,C4′) (CrtD) and methoxylation at C1, C1′ (CrtF).

To produce acyclic C₃₀ xanthophylls in engineered E. coli cells, the diapolycopene pathway was extended in E. coli pAC-crtM(SA)-crtN(SA) with crtA on pUC-crtA(RC). The co-transformed cells appeared more yellow than E. coli pAC-crtM(SA)-crtN(SA). HPLC analysis of the cell extract showed three new very polar peaks (FIG. 2C). The absorption maxima and spectral fine structure of the major carotenoid corresponds to an acyclic carotenoid without conjugated carbonyl-functions and with eight conjugated double bonds as opposed to the nine conjugated double bonds in diaponeurosporene (FIG. 1). The two minor peaks showed spectral properties similar to diapo-ζ-carotene and diapophytoene. Further structural analysis of the yellow carotenoid by HPLC-mass spectrometry showed an unexpected molecular mass of m/z 536.3 along with the prominent [M-18]⁺ (loss of a hydroxy-group) and [M-58]⁺, [M-87]⁺ ions (loss of an end-group adjacent to a keto-group), indicating a putative C₃₅ backbone structure rather than C₃₀. Further fragmentation of the parent ion by MS/MS analysis gave additional unique [M-18-16]⁺ (loss of oxygen from carbonyl group) and [M-18-28]⁺ ions (loss of carbonyl group). Although these fragmentation patterns are consistent with expected CrtA end-group monooxygenase activity, the high overall mass suggests a non-specific activity or unknown biocatalytic function of CrtA. The APCI mass spectrum of the putative C35 carotenoid is shown in FIG. 2F.

### Example 5

### Spheroidene Monooxygenase CrtA Oxygenizes Acyclic Intermediates of the Phytoene (C40) Desaturation Pathway

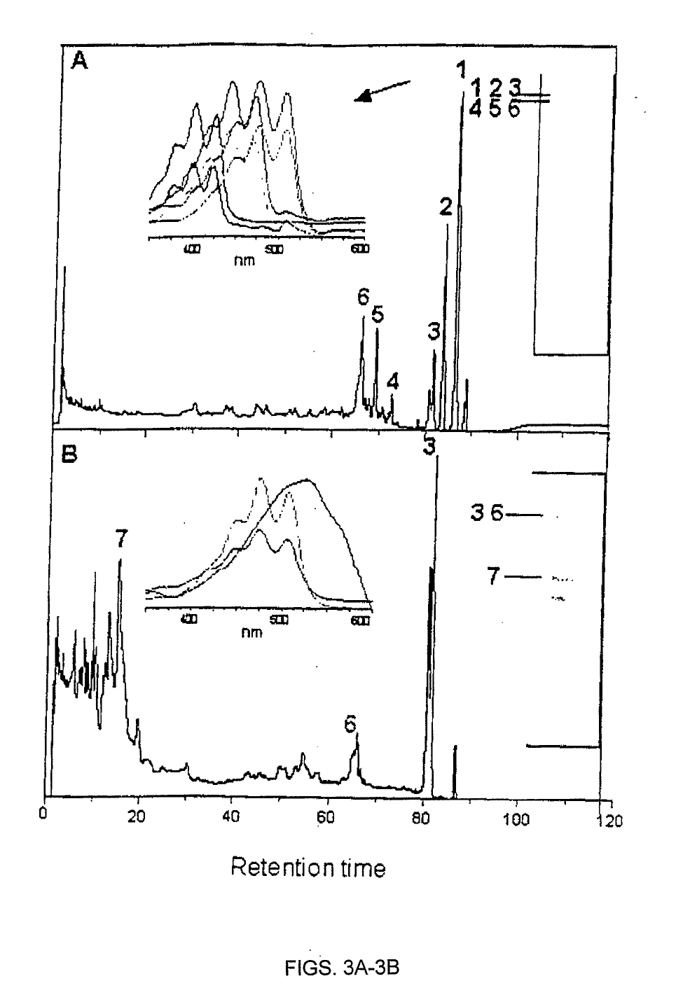

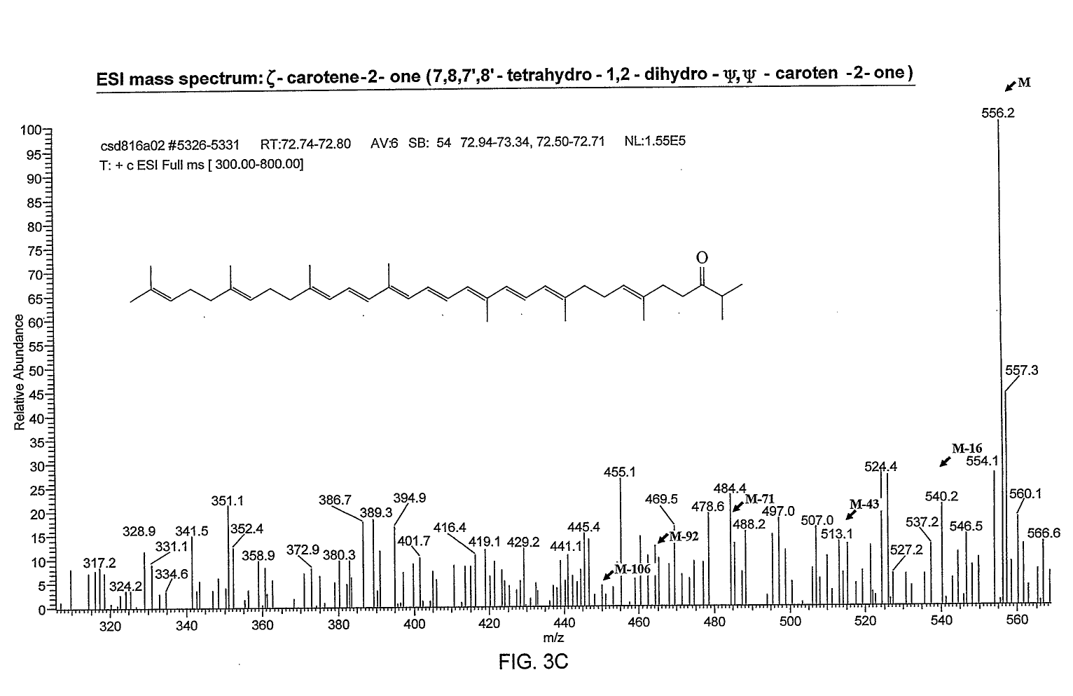

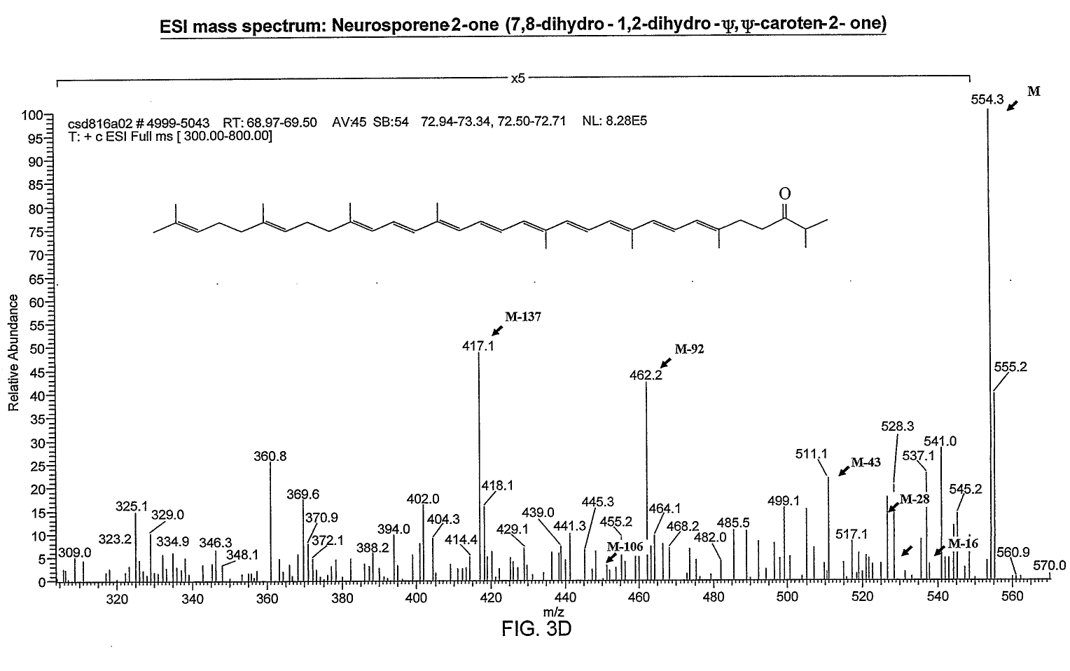

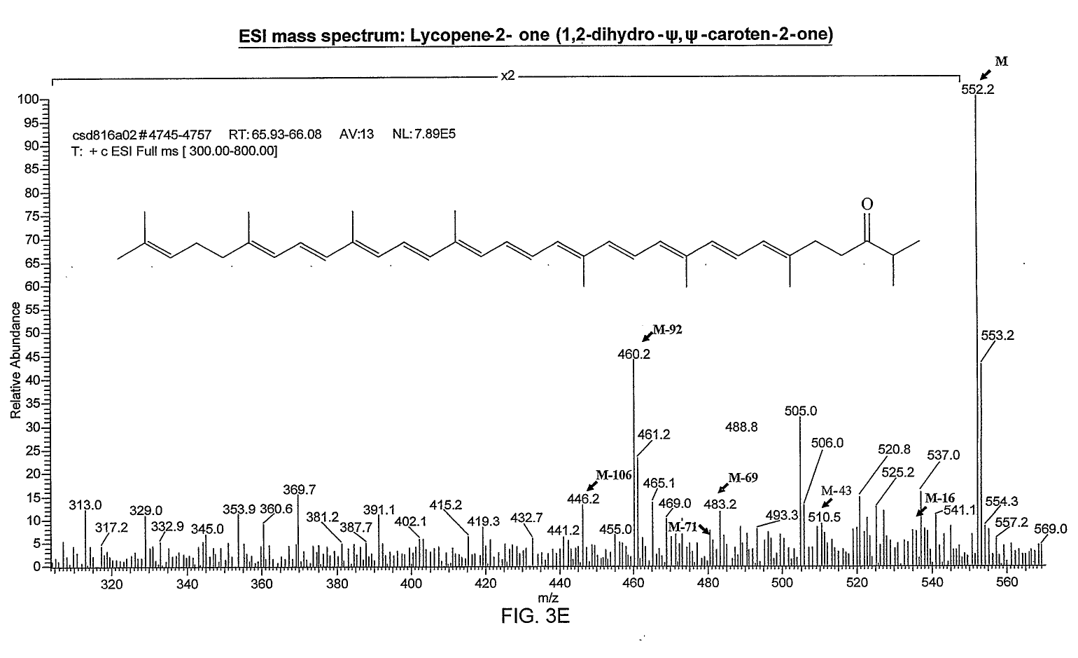

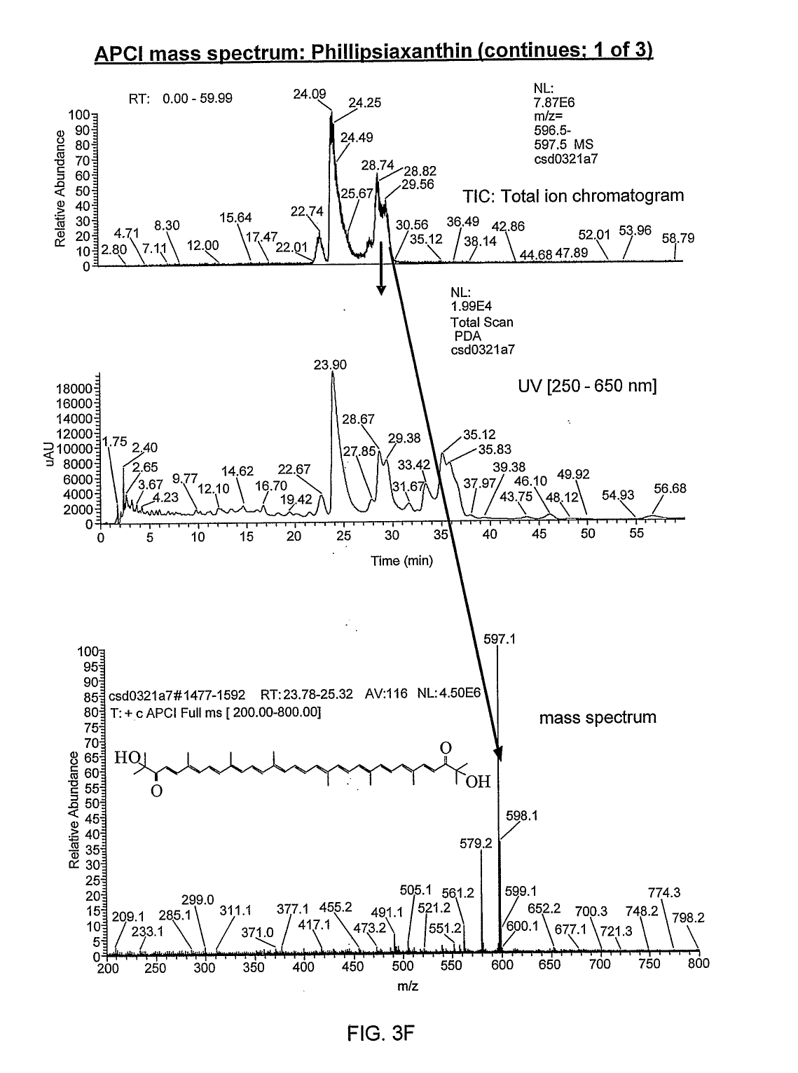

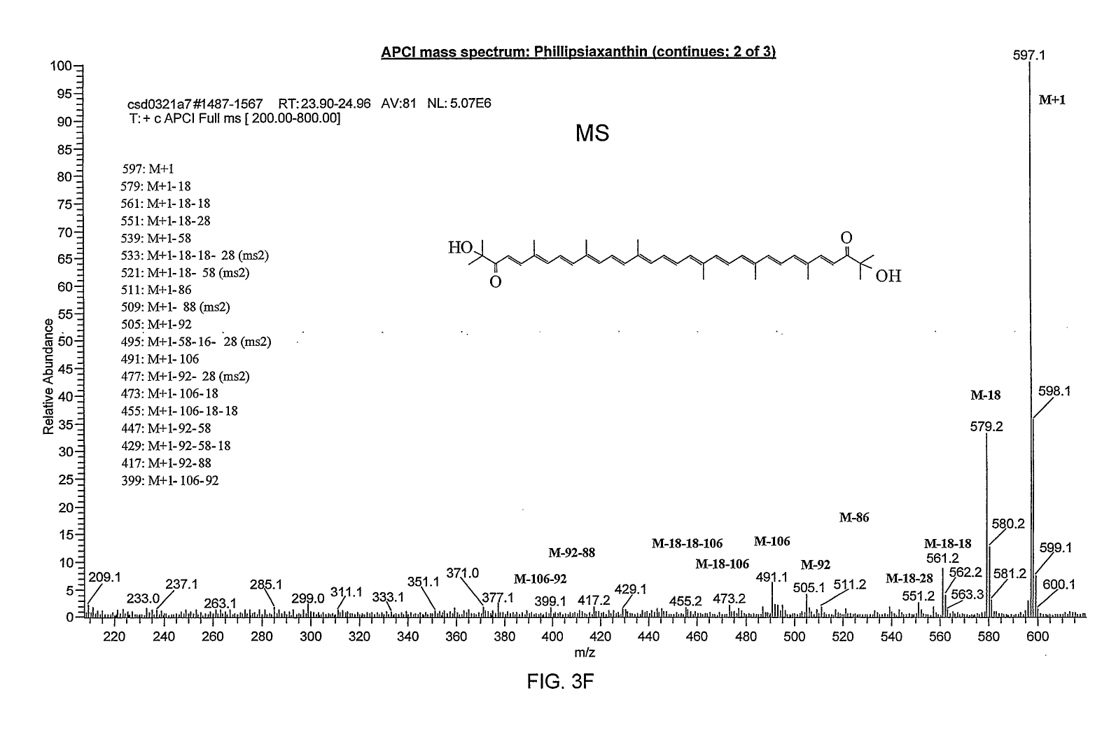

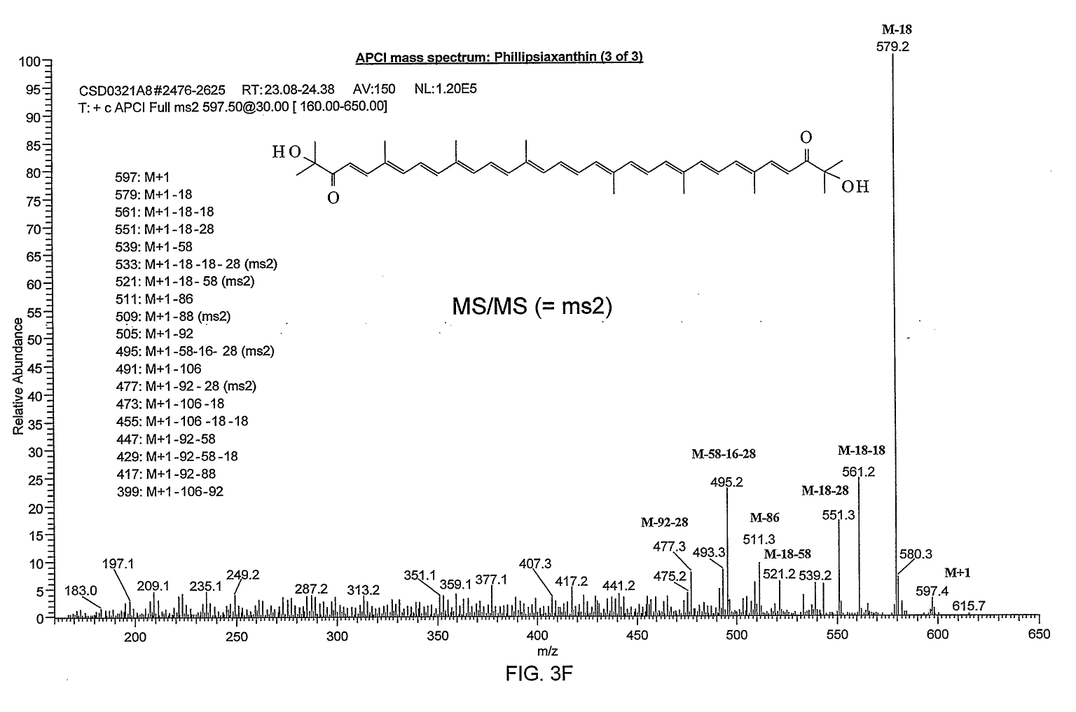

In order to generate new, acyclic, purple C₄₀ xanthophylls in E. coli from the wild-type lycopene and in vitro evolved tetradehydrolycopene biosynthetic pathways, CrtA was applied to introduce keto-groups and thus extend the chromophore of these products. When lycopene or tetradehydrolycopene-accumulating E. coli cells harboring pAC-crtE(EU)-crtB(EU)-crtI(EU) (orange-red cells) or pAC-crtE(EU)-crtB(EU)-crtI₁₄ (pink cells) (see Schmidt-Dannert et al. (2000) supra) were co-transformed with pUC-crtA(RC), the cell color changed to yellow and deep red, respectively. All carotenoid extracts were separated by high-performance thin layer chromatography (HP-TLC) and high-pressure liquid chromatography (HPLC) (FIG. 3), and structural identification was achieved by considering their polarity, absorption properties, and mass fragmentation patterns (compared to fragmentation patterns of known carotenoid end-groups). Extension of the lycopene pathway by coexpression of pUC-crtA(RC) with pAC-crtE(EU)-crtB(EU)-crtI(EU) in E. coli resulted in the synthesis of three novel acyclic xanthophylls ζ-carotene-2-one (7,8,7′,8′-tetrahydro-1,2-dihydro-ψ,ψ-caroten-2-one), neurosporene-2-one (7,8-dihydro-1,2-dihydro-ψ,ψ-caroten-2-one) and lycopene-2-one (1,2-dihydro-ψ,ψ-caroten-2-one) (FIG. 3A). ESI mass spectra for 1-carotene-2-one, neurosporene-2-one, and lycopene-2-one are shown in FIG. 3C-3E. Unexpectedly, the yellow carotenoids ζ-carotene and neurosporene, undetectable intermediates in lycopene producing E. coli pAC-crtE(EU)-crtB(EU)-crtI(EU), also accumulated, indicating that CrtA uncouples the desaturation sequence catalyzed by CrtI. In addition, several minor more polar compound peaks were observed after HPLC separation. These compounds showed absorption characteristics of lycopene and neurosporene but with masses corresponding to the respective diketo- and dihydroxy-diketo-derivatives. A deep purple dihydroxy-diketo-derivative of tetradehydrolycopene identified as phillipsiaxanthin (chemical synthesis and mass fragmentation described in Schwieter et al. (1966) Helv. Chim. Acta 49:992-996) and lycopene constitute the major carotenoids synthesized by E. coli pAC-crtE(EU)-crtB(EU)-crtI₁₄ co-expressing pUC-crtA(RC). The APCI mass spectrum of phillipsiaxanthin is shown in FIG. 3F. Lycopene-2-one was accumulated as a minor product along with other polar xanthophylls that could not be identified unequivocally (FIG. 3B).

This examples indicates that co-expression of CrtA with acyclic C40 carotenoid pathways can introduce a keto-group at the C(2, 2′) position of unnatural substrates that do not exhibit a C(3,4) double bond, which was previously thought to be necessary (Britton (1998) Carotenoids: Biosynthesis and Metabolism, Vol. 3, G. Britton, ed. (Basel: Birkhäuser), pp. 13-147). In addition, the complete conversion of tetradehydrolycopene to phillipsiaxanthin observed (FIG. 3B), suggests it is a favorable substrate for CrtA activity when compared to the incomplete conversion of lycopene to lycopene-2-one in the presence of CrtA.

### Example 6

### β-Carotene Oxygenase CrtO Introduces Keto-Groups in Torulene and β,β-Carotene

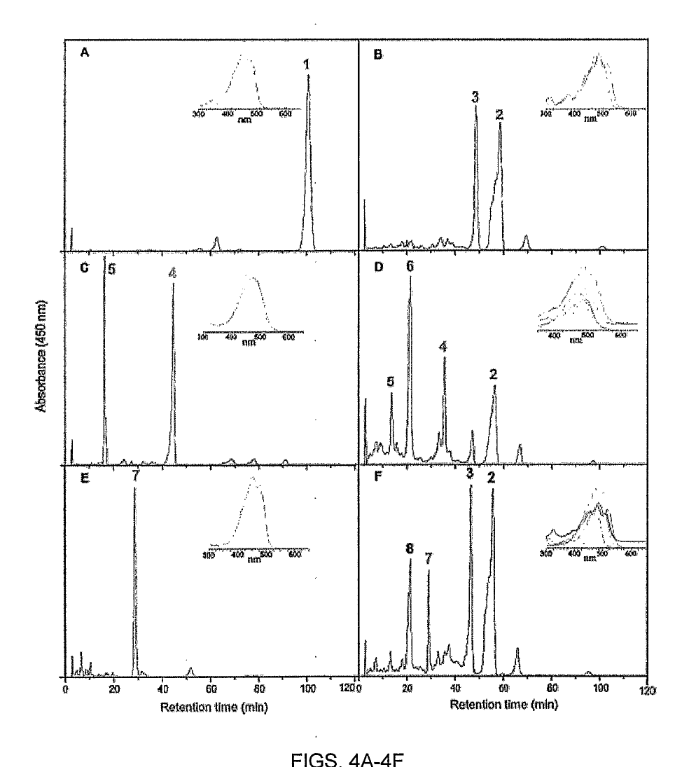

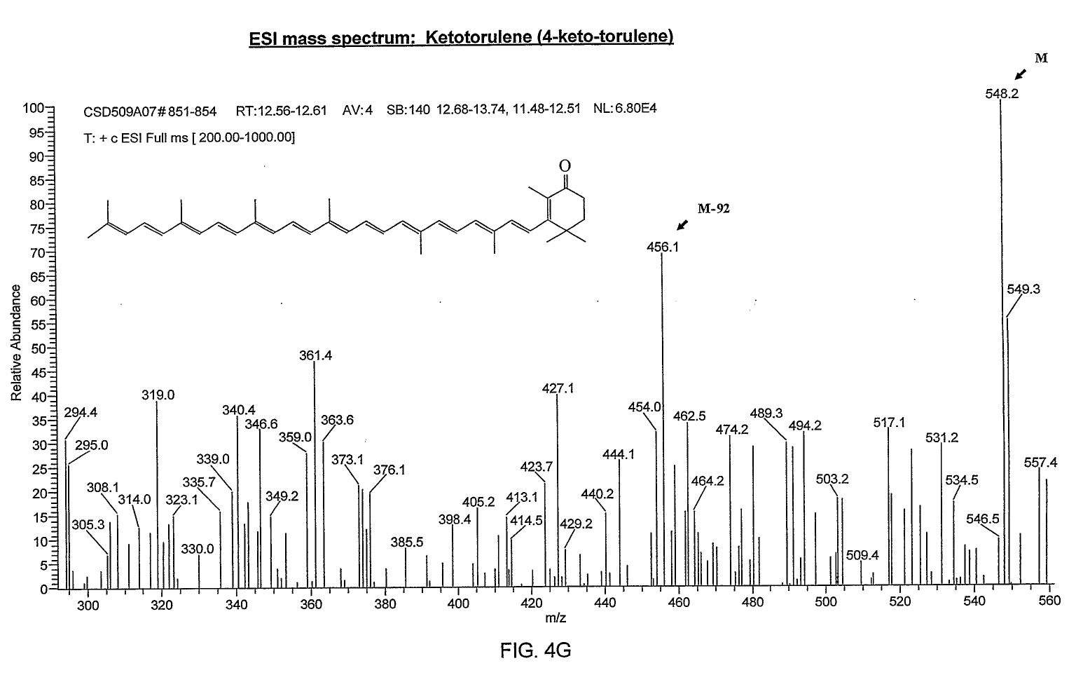

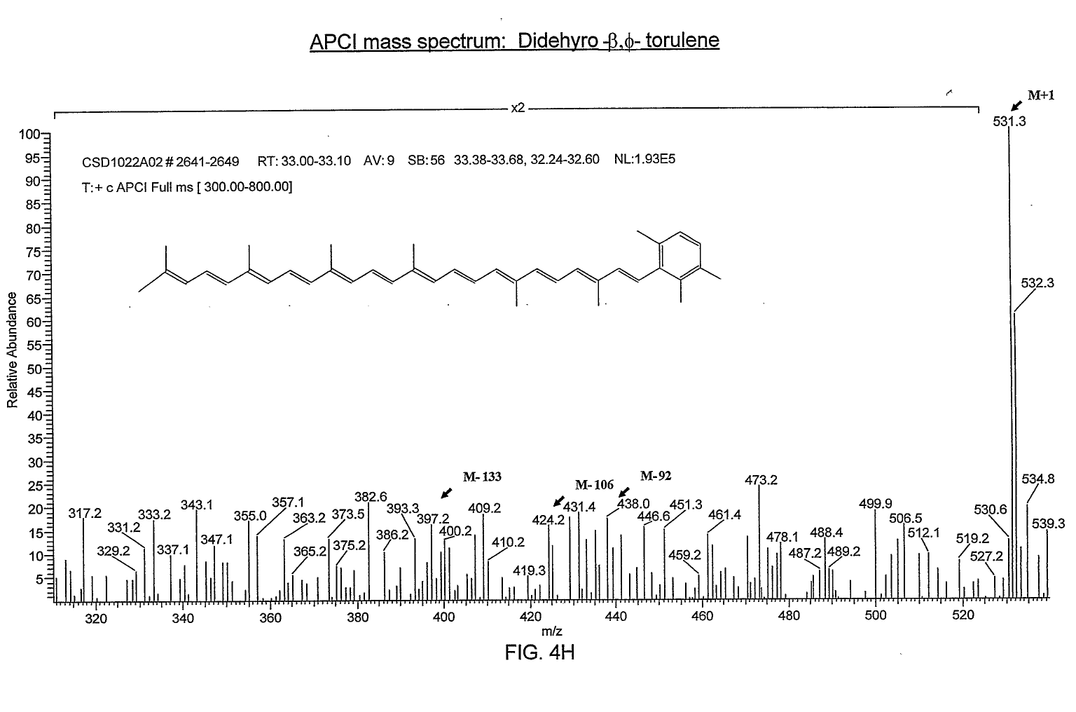

The catalytic promiscuity of different cloned β,β-carotene modifying enzymes towards torulene was probed for the production of novel cyclic carotenoids. To extend the evolved torulene and, as a control, the wild-type β,β-carotene pathway, with different carotenoid genes in E. coli, the lycopene cyclase crtY(EU) or evolved cyclase crtY2(EU/EH) genes were cloned into pAC-crtE(EU)-crtB(EU)-crtI₁₄ to yield pAC-crtE(EU)-crtB(EU)-crtI₁₄-crtY(EU) and pAC-crtE(EU)-crtB(EU)-crtI₁₄-crtY2(EU/EH). E. coli cells harboring pAC-crtE(EU)-crtB(EU)-crtI₁₄-crtY(EU/EH) developed a bright orange color due to the synthesis of β,β-carotene, while E. coli cells transformed with pAC-crtE(EU)-crtB(EU)-crtI₁₄-crtY2(EU/EH) turned bright red due to the production of torulene and lycopene (FIGS. 4A, B).

The introduction of keto-groups at position C4(4′) of one or both rings of β,β-carotene is catalyzed by β-carotene oxygenases or ketolases. Most β-carotene oxygenases show homology to fatty acid desaturases and introduce keto-groups at both β-rings to synthesize canthaxanthin, the precursor of the biotechllologically important carotenoid astaxanthin (FIG. 1). However, β-carotene oxygenase CrtO from Synechocystis sp. is unique as it shows high homology to phytoene dehydrogenases and has been reported to introduce only one keto-group at C4 of one Bring, as present in torulene, to synthesize echinenone. E. coli pAC-crtE(EU)-crtB(EU)-crtI₁₄-crtY(EU) or pAC-crtE(EU)-crtB(EU)-crtI₁₄-crtY2(EU/EH) expressing the β,β-carotene or torulene pathways, respectively, were co-transformed with pUC-crtO(SS). Surprisingly, in this system where each carotenoid enzyme is individually expressed under the control of a constitutive lac-promoter, CrtO introduced keto-groups efficiently at both rings of β,β-carotene to yield canthaxanthin in a similar ratio to the mono-keto product echinenone (FIG. 4C). The symmetrical activity of CrtO on β,β-carotene was not related to the gene copy number of crtO(SS) on pUC-crtO(SS) as similar ratios of canthaxanthin and echinenone were produced by E. coli with the single plasmid system pAC-crtE(EU)-crtB(EU)-crtI₁₄-crtY(EU)-crtO(SS). Analysis of extracts from cells expressing the CrtO extended torulene pathway, however, revealed synthesis of a new, major carotenoid in addition to smaller amounts of echinenone, canthaxanthin, torulene and lycopene (FIG. 4D). Absorption maxima, polarity and mass fragmentation spectrum of this new carotenoid identified it as 4-keto-torulene (FIG. 1). The ESI mass spectrum for ketotorulene is shown in FIG. 4G.

### Example 7

### Aromatic Carotenoids are Produced from β,β-Carotene and Torulene by CrtU

Aromatic carotenoids have been isolated from several bacteria and three bacterial β-carotene desaturases (CrtU) have recently been cloned and characterized in their homologous hosts. See Krugel et al. (1999) Biochim. Biophys. Acta 1439, 57-64; Krubasik and Sandmann (2000). Mol. Gen. Genetics 263, 423-432; and Viveiros et al., (2000) FEMS Microbiol. Lett. 187, 95-101). The symmetrical aromatization of β,β-carotene to isoreneriatene (φ,φ-carotene) by CrtU involves the introduction of two double bonds and a concurrent methyl group shift for each β-ring (FIG. 1). It was first examined whether CrtU can function cooperatively with other heterologous carotenoid enzymes in engineered E. coli.

The exclusive formation of isorenariatene by E. coli pAC-crtE(EU)-crtB(EU)-crtI₁₄-crtY(EU) co-expressed with pUC-crtU(BL) proved therefore that CrtU functions cooperatively with other carotenoid enzymes assembled from different organisms (FIG. 4E). When E. coli cells harboring pAC-crtE(EU)-crtB(EU)-crtI₁₄-crtY2(EU/EH) were co-transformed with pUC-crtU(BL), a new, more polar major carotenoid accumulated along with isoreniaratene, lycopene and torulene (FIG. 4F) and was identified by adsorption maxima, polarity and mass fragmentation spectrum as aromatic torulene (didehydro-β,φ-carotene). The APCI mass spectrum of didehydro-β,φ-carotene is shown in FIG. 4H.

### Example 8

### β-Carotene Hydroxylase CrtZ and Zeaxanthin Glucosylase CrtX Produce Novel Torulene Derivatives

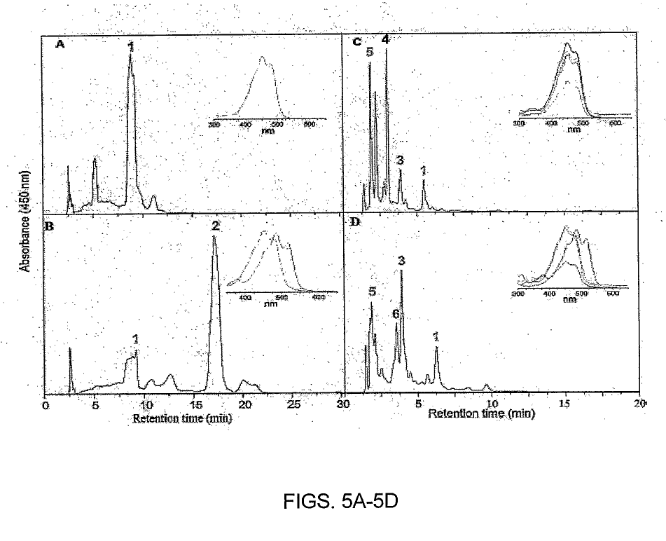

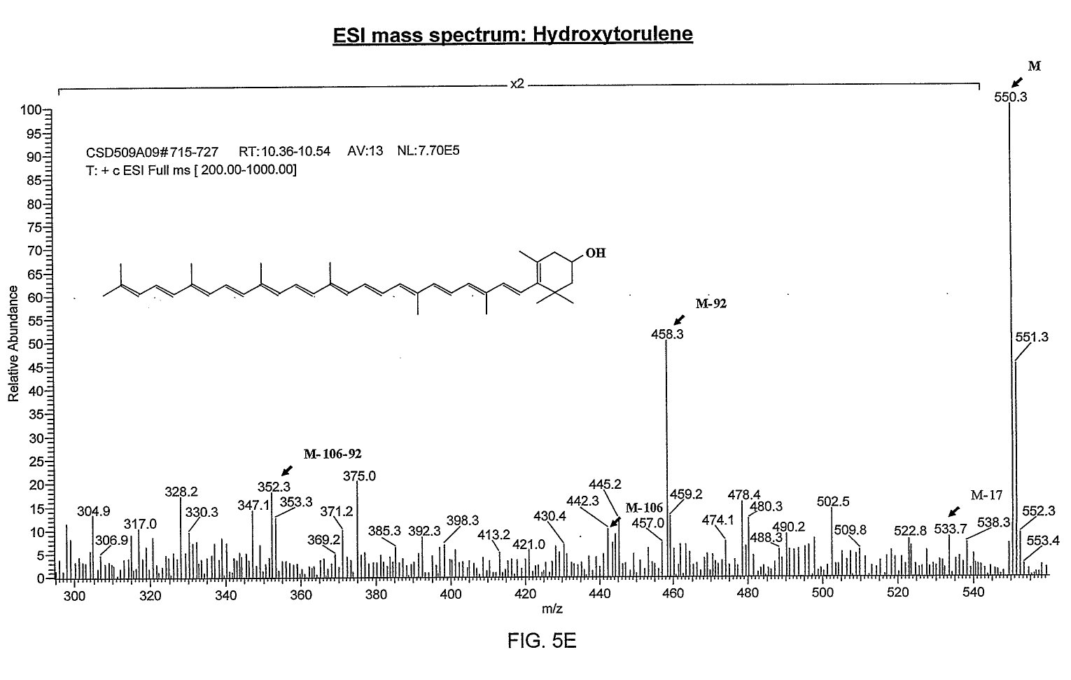

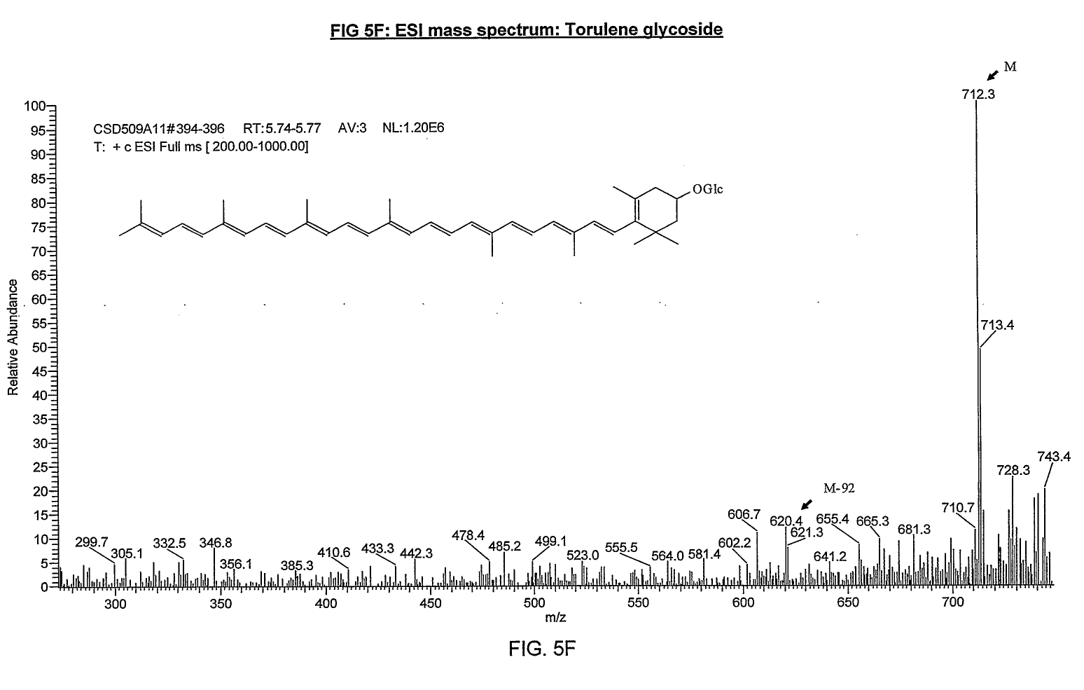

The catalytic promiscuity observed for CrtO and CrtU with torulene suggested that β-carotene hydroxylase CrtZ and zeaxanthin glucosylase CrtX, which converts β,β-carotene to the highly polar zeaxanthin-diglucoside in e.g. Erwinia strains (FIG. 1), may exhibit similar broad substrate specificities and allow synthesis of a novel polar torulene-glucoside in E. coli. To extend the torulene and, as a control, the β,β-carotene biosynthesis pathway in E. coli with the two enzymes (CrtZ and CrtX) necessary for β-ring glucosylation, crtZ was cloned into pAC-crtE(EU)-crtB(EU)-crtI₁₄-crtY(EU/EH) (β,β-carotene) and pAC-crtE(EU)-crtB(EU)-crtI₁₄-crtY2(EU/EH) (torulene) to create pAC-crtE(EU)-crtB(EU)-crtI₁₄-crtY(EU)-crtZ(EU) and pAC-crtE(EU)-crtB(EU)-crtI₁₄-crtY2(EU/EH)-crtZ(EU). Pathway extension with CrtZ resulted in the symmetrical hydroxylation of β,β-carotene to zeaxanthin, which was formed as the only product in E. coli pAC-crtE(EU)-crtB(EU)-crtI₁₄-crtY(EU)-crtZ(EU) (FIG. 5A). However, a new polar carotenoid, with an absorption spectrum similar to torulene but with a mass spectrum expected for hydroxytorulene, accumulated as the main product in E. coli pAC-crtE(EU)-crtB(EU)-crtI₁₄-crtY2(EU/EH)-crtZ(EU) (FIG. 5B) suggesting that torulene and β,β-carotene are equally good substrates for CrtZ. ESI mass spectrum for hydroxytorulene is shown in FIG. 5E. Subsequent combination in E. coli of pAC-crtE(EU)-crtB(EU)-crtI₁₄-crtY2(EU/EH)/crtY(EU)-crtZ(EU) together with the terminal enzyme CrtX of the glucosylation pathway expressed on pUC-crtX(EH), gave rise to a number of very polar carotenoid structures in E. coli. The assembled β,β-carotene glucosylation pathway in E. coli harboring pAC-crtE(EU)-crtB(EU)-crtI₁₄-crtY(EU)-crtZ(EU) and pUC-crtX(EH) produced zeaxanthin-diglucoside as a major product. Other biosyiithesis intermediates such as zeaxanthin, zeaxanthin-monoglucoside, β-cryptoxanthin-monoglucoside (one β-ring of β,β-carotene glucosylated) were also produced (FIG. 5C). Neither hydroxytorulene nor its precursor torulene accumulated in E. coli cells carrying the assembled torulene glycosylation pathway, but a new carotenoid identified as torulene glucoside is synthesized in addition to different hydroxylated and glucosylated β,β-carotene derivatives (FIG. 5D). The ESI mass spectrum of torulene glucoside is shown in FIG. 5F. The formation of carotenoids where only one β-ring is hydroxylated or glucosylated, indicates that CrtZ and CrtX catalyze β-ring modification irrespective of the other end-structure present in a carotenoid molecule.

### Example 9

### Metabolic Engineering of the Methylotropic Yeast Pichia pastoris for Enhanced Carotenoid Production

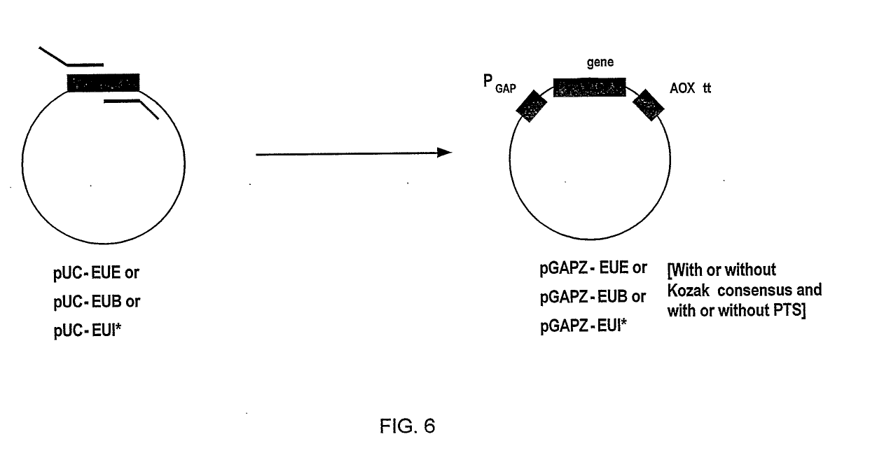

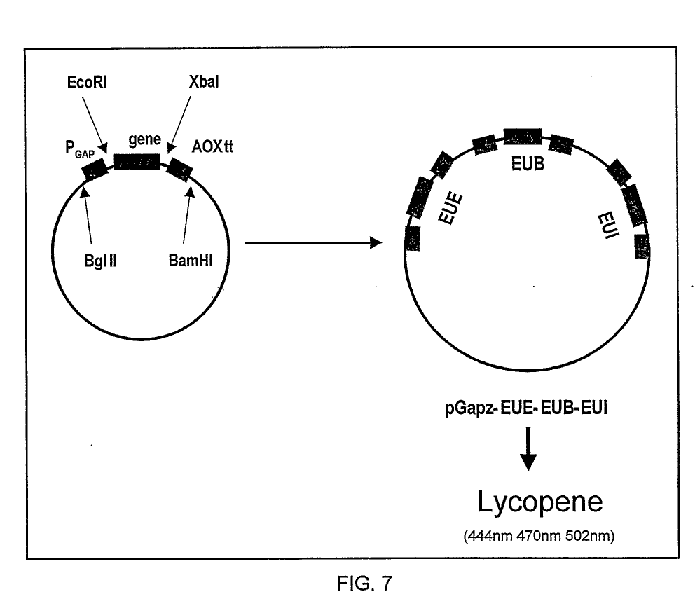

Heterologous carotenoid genes required for lycopene (crtE, crtB, crtI), tetradehydrolycopene (crtE, crtB, crtI₁₄), “,”-carotene (crtE, crtB, crtI, crtY), and torulene (crtE, crtB, crtI₁₄, crtY2) production by extension of the general yeast isoprenoid pathway were subcloned (FIG. 6) into a multi-copy integration E. coli-Pichia shuttle vector (pGAPZ, Invitrogen) bearing a functional constitutive GAP-promoter and a terminator. All expression cassettes were then assembled on a single vector (FIG. 7). After purification from E. coli, the plasmid was transformed into P. pastoris, and carotenoid producing variants were selected. Production levels were compared between clones with peroxisomal targeting of proteins and those without targeting. For subsequent product analysis various extraction procedures were compared and even modified to optimize extraction of carotenoid from P. pastoris.

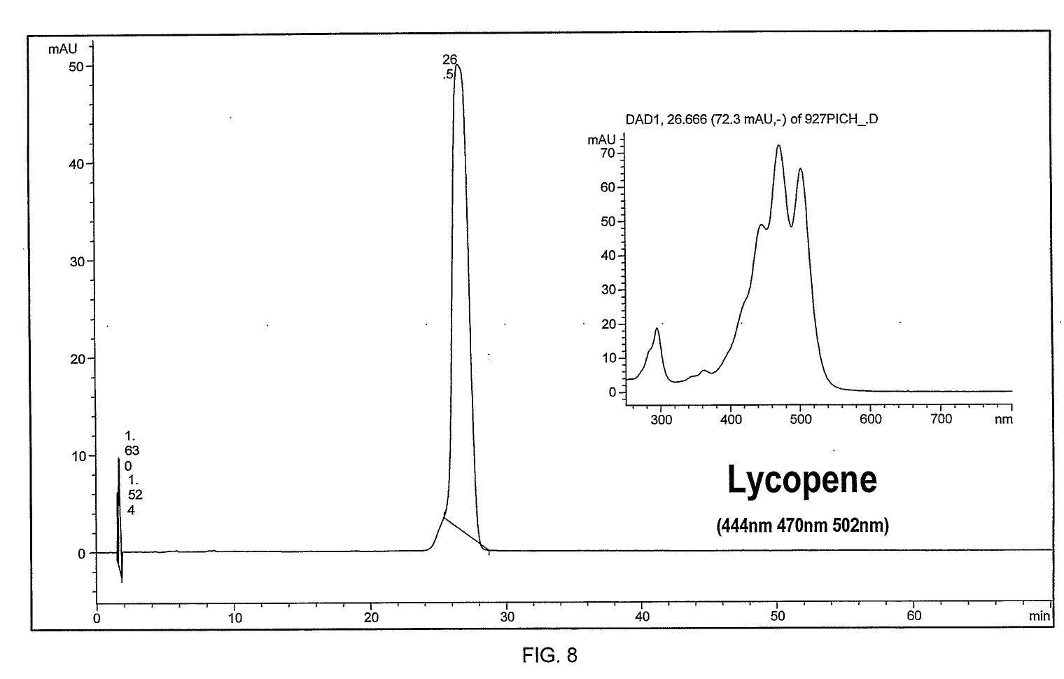

Carotenoid production of the four example carotenoid pathways were analyzed and quantified by UV-visual spectra, thin layer chromatography and high performance liquid chromatography. Carotenoid levels in the range of several mg of carotenoids per gram of dry cell weight were obtained in P. pastoris. FIG. 8 shows the HPLC analysis of the carotenoid extract in recombinant P. pastoris.

### Example 10

### Production of Acyclic Carotenoids Using Oxygenases

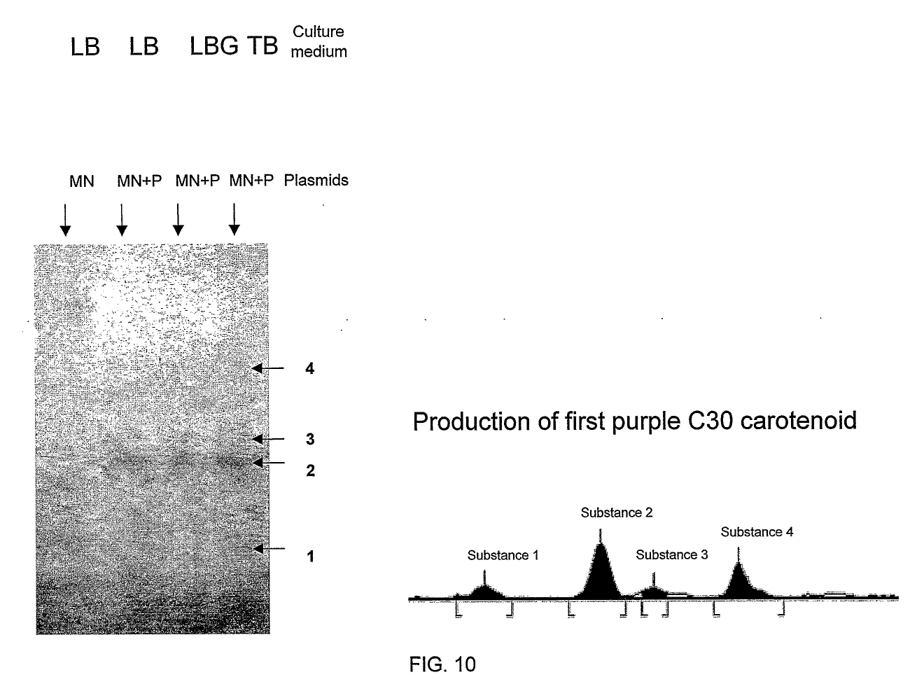

Recombinant E. coli cells expressing diapophytoene synthase crtN and diapophytoene desaturase crtM from Staphylococcus and producing diaponeurosporene and diapolycopene were co-transformed with newly discovered carotenoid oxygenase sequences identified in the genomes of Staphylococcus and Oceanobacillus (see Table 2). E. coli cells co-expressing the C30 carotenoid pathway together with carotenoid oxegenases from these organisms turned purple due to the production of C30 carotenoids containing terminal aldehyde and carboxyl functions. FIG. 9 shows the pathway leading to these compounds. FIG. 10 shows examples of purple carotenoids extracted from engineered E. coli cells. The discovered carotenoid oxygenases also can be used to oxidize the acyclic ends of other C30 and C40 carotenoid structures (for example, lycopene, neurosporene, didehydrolycopene and torulene) to produce a variety of novel carotenoid aldehydes and carotenoid carboxylic acids.

TABLE 2
Oxidoreductases and crtM and crtN sequences
identified in microbial genome sequences.

| Enzyme | Activity | Source | Accession No. |
|---|---|---|---|
| IspA | FPP synthase | E. coli | AAC73524 |
| CrtM | 4,4′-diapophytoene synthase | S. aureus | A55548 |
| CrtN | 4,4′-diapophytoene desaturase | S. aureus | B55548 |
| CrtOx | 4,4′-diapocarotene oxygenase | S. aureus | NP_373088 |
| CrtGT | putative 4,4′-diapocarotene glycosyl | S. aureus | NP_373087 |
| | transferase | | |
| CrtAT | putative 4,4′-diapocarotene acyl | S. aureus | NP_373089 |
| | transferase | | |
| CrtM | 4,4′-diapophytoene synthase | O. iheyensis | NP_693381 |
| CrtN | 4,4′-diapophytoene desaturase | O. iheyensis | NP_693382 |
| CrtOx | 4,4′-diapocarotene oxygenase | O. iheyensis | NP_693380 |
| CrtGT | putative 4,4′-diapocarotene glycosyl | O. iheyensis | NP_693379 |
| | transferase | | |
| CrtAT | putative 4,4′-diapocarotene acyl | O. iheyensis | NP_693378 |
| | transferase | | |
| CrtOx | 4,4′-diapocarotene oxygenase | Exiguobacterium | ZP_00183789 |
| | | sp. 255-15 | |

### Methods and Materials

### Bioinformatics

Whole genome DNA sequences of S. aureus strains MW2 (NC_{—}003923), N315 (NC_{—}002745), and Mu50(NC_{—}002758) and Oceanobacillus iheyensis (NC_{—}004193) were obtained from NCBI. Protein sequences of S. aureus CrtN (crtN(SA); B55548) and CrtM (crtM(SA); A55548) were obtained from NCBI. Homology searches were performed using NCBI BLAST software. Altschul et al. (1990) J. Mol. Biol. 215:403-410. Genome region analysis and ORF prediction were performed using TIGR Comprehensive Microbial Resource (Peterson et al., (2001) Nucleic Acids Res. 29:123-5). Sequence editing was performed using Bioedit software (Hall (1999) Nucl. Acids Symp. Ser. 41:95-98). Enzyme activities and GenBank Accession numbers are provided in Table 2 (above).

### Strains and Culture Conditions

All cloning and DNA manipulations were carried out in E. coli JM109 using standard techniques (Sambrook et al., Molecular cloning: a laboratory manual, Cold Spring Harbour Laboratory Press, New York, USA, second edition (1989)) and unless otherwise stated, microorganisms were grown at 30° C. with shaking at 300 RPM. Following sequencing, plasmids were transformed into E. coli strain JM109 for expression (Table 3). S. aureus (ATCC 35556D) genomic DNA was acquired from the ATCC, O. iheyensis was acquired from DSMZ and cultured in PY medium (Lu et al. (2001) FEMS Microbiol. Lett. 205:291-9) for 48 hours at room temperature with shaking at 300 RPM. Genomic DNA was prepared using a Promega Wizard SV genomic DNA kit.

TABLE 3
Strains and plasmids.

| Strain or Plasmid | Properties or Genotype | Source |
|---|---|---|
| Strains | | |
| E. coli JM109 | e14⁻(McrA⁻) recAl endAl gyrA96 thi-1 | Yanisch-Perron |
| | hsdR17 (r_{K}⁻m_{K}⁺) supE44 relAl A(lac | et al. (1985) |
| | proAB) [F' traD36 proAB lacl^{q}ZΔM15] | Gene 33: 103-19. |
| O. iheyensis HTE831 | Type Strain | DSMZ |
| (DSMZ 14371) | | |
| S. aureus SA113 | Type Strain | ATCC |
| (ATCC 35556) | | |
| Plasmids | | |
| pAC-ispA(EC)-crtM(SA)- | Constitutively expressed E. coli IspA, | Lee et al., |
| crtN(SA) | S. aureus CrtM and S. aureus CrtN | (2003) Chem. |
| | | Biol. 10: 453-62. |
| pAC-ispA(EC)-crtM(OI)- | Constitutively expressed E. coli IspA, | |
| crtN(OI) | O. iheyensis CrtM, and O. iheyensis CrtN | |
| pUC-crtOx(SA) | Constitutively expressed S. aureus CrtOx | |
| pUC-crtOx(OI) | Constitutively expressed O. iheyensis CrtOx | |

### Plasmid Construction

Cloning of the S. aureus carotenoid pathway genes CrtN and CrtM is described herein. The cloning of the E. coli prenyltransferase IspA and construction of the plasmid pAC-ispA(EC)-crtM(SA)-crtN(SA) have been described by Lee et al., (2003) Chem. Biol. 10:453-62. The S. aureus carotenoid gene CrtOx was amplified from S. aureus (ATCC 35556D) genomic DNA using PCR primers SA1Ox-f-X (5′-GCTCTAGAAGGAGGATT-ACAAAATGACTAAACATATCATCG-3′, SEQ ID NO:2) and SA1Ox-f-N (5′-TTC-CTTTGCGGCCGCTCACTTCCTATTCTTCGC-3′, SEQ ID NO:3). The homologous gene from O. iheyensis was amplified from O. iheyensis genomic DNA using the PCR primers OIOxF XbaI (5′-GCTCTAGAAGGAGGTGAATAACATGAAAAAGGTAAT-TAT-3′, SEQ ID NO:4) and OIOxR NotI (5′-TTCCTTTGCGGCCGCCCTTAAC-ATTAACTAAATATCTGAT-3′, SEQ ID NO:5). These PCR products were digested with XbaI and NotI enzymes and gel purified and ligated into similarly prepared pUCMod vector (described herein) to yield pUC-crtOx(SA) and pUC-crtOx(OI), respectively. Insert containing plasmids were isolated and sequenced to confirm no PCR errors were present. The two additional S. aureus carotenoid pathway genes CrtGT and CrtAT were amplified from genomic DNA using PCR primer pairs SAGTF_XbaI (5′-GCTCTAGAAGGAGGATTACAAAATGAAATGGTTATCACGAATAT-3′, SEQ ID NO:6), SAGTR_NotI (5′-TTCCTTTGCGGCCGCCCTTGATTTATTGTTCTT-3′, SEQ ID NO:7) and SAXYF_XbaI (5′-GCTCTAGAAGGAGGATTACAAAATGAAAACC-ATGAAAAAATATA-3′, SEQ ID NO:8), SAXYR_NotI (5′-TTCCTTTGCGGCCGC-TTAGTCATGACGTTCAC-3′, SEQ ID NO:9), respectively. Following digestion of the PCR products with XbaI and NotI, the genes were cloned into similarly prepared pUCmod to yield pUC-crtGT(SA) and pUC-crtAT(SA), respectively.

O. iheyensis homologues of the genes CrtM and CrtN present in the O. iheyensis genomic operon were PCR amplified as a contiguous DNA fragment using the primers OIN XbaI_F (5′-GCTCTAGAAGGAGGATGTCTATGAAAA-3′, SEQ ID NO:10) and OIM_NotI_R (5′-TTCCTTTGCGGCCGCTAGATACTAGTAGCTTGA-3′, SEQ ID NO:11) and cloned into the pUCMod vector as above. E. coli JM109 strains harboring this plasmid produced a yellow pigmented phenotype. A contiguous O. iheyensis crtM-crtN DNA fragment was then PCR amplified using the PCR primers pUCinR_SalI (5′-GACGCGTCGACATATGCGGTGTGAAATACCG-3′, SEQ ID NO:12) and pUCInF_SphI (5′-GACGCGCATGCCCGACTGGAAAGCGG-3′, SEQ ID NO:13) and subcloned into the pACMod vector to produce pAC-crtM(OI)-crtN(OI). This vector was then digested with SphI and SalI and ligated into similarly digested pAC-IspA(EC) vector to produce pAC-IspA(EC)-crtM(OI)-crtN(OI).

### Carotenoid Expression and Optimization

Initially, production of novel C30 carotenoids was attempted by co-expressing the plasmids pAC-ispA(EC)-crtM(SA)-crtN(SA) and pUC-crtOx(SA) in E. coli strain JM109. Recombinant cells were cultured at 37° C., 300 RPM in LB medium supplemented with carbenicillin and chloramphenicol. In order to optimize carotenoid production for this strain, cultures were grown in LB medium, LB supplemented with 0.5% glycerol (LBG), and TB medium at 30 and 37° C. for 24 hours. E. coli strains harboring pAC-ispA (EC)-crtM(SA)-crtN(SA)/pUC-crtOx(OI), pAC-IspA(EC)-crtM(OI)-crtN(OI)/pUC-crtOx(OI), and pAC-IspA(EC)-crtM(OI)-crtN(OI)/pUC-crtOx(SA) were cultured under optimized conditions and carotenoids extracted by acetone and analyzed by TLC.

### Carotenoid Extraction and Purification

For analytical identification of the novel carotenoids produced, JM109 (pAC-ispA(EC)-crtM(SA)-crtN(SA)/pUC-crtOx(SA)) was cultured at 30° C. in 500 mL LBG medium for 24 hours and cells pelleted. Carotenoids were extracted by addition of 15 mL of acetone to cell pellets and incubation in a sonicating water bath at 4° C. for 30 minutes, followed by centrifugation to remove cell debris. Extraction with acetone was repeated until no pigment was visible in the cell pellets and the supernatants pooled. Pooled extracts were dried down completely under a stream of N₂ gas and resuspended in 20 mL of hexanes. A precipitate that formed upon hexane resuspension was pelleted by centrifugation, dried and resuspended in 20 mL ethyl acetate. Both samples were two-phase extracted with 20 mL 5M NaCl, solvent phases recovered, dried down and resuspended in 2 mL of acetone. The hexane fraction was then loaded onto silica gel open columns developed with hexanes followed by mixtures of hexanes with increasing acetone concentrations (10, 25, and 50% acetone). The ethyl acetate fraction was similarly developed using a starting mobile phase of 80% hexanes, 20% acetone followed by 50% acetone, 50% hexanes. Like fractions from each column preparation were pooled, dried down and stored at −80° C.

### Time Course Study

JM109 (pAC-ispA(EC)-crtM(SA)-crtN(SA)/pUC-crtOx(SA)) was cultured at 30° C. in 500 mL LBG medium and 20 mL culture samples collected at 24 hour intervals for 144 hours. Samples were centrifuged when collected, supernatants discarded and pellets stored at −20° C. until analysis. Pigments in cell pellets were repeatedly extracted with 2 mL acetone as above until no additional pigment was visible in the acetone supernatant. Acetone fractions were dried down under a stream of N₂ gas, resuspended in 5 mL ethyl acetate, and washed with 5 mL salt water. The ethyl acetate was then dried under N₂ gas and samples resuspended in 1 mL methanol for analysis.

### HPLC and LC-MS Analysis

Pooled fractions from silica gel chromatography above were analyzed by TLC as described previously and by HPLC and LC-MS. HPLC separation was performed using a Zorbax SB-C18 column (4.6×250 mm, 5 μM; Agilent technologies, Palo Alto, Calif.) with 100% MeOH as an isocratic mobile phase at a flow rate of 1 mL min⁻¹ using an Agilent 1100 HPLC system equipped with a photodiode array detector. Mass spectrometry was performed under the same conditions as HPLC analysis. Mass spectra were monitored in a mass range of m/z 200-800 or 1000 on a LCQ mass spectrophotometer equipped with an atmosphere pressure chemical ionization (APCI) interface (Thermo Finnigan, USA).

### Saponification and Extraction of Aqueous Pigments

Cell pellets from 500 mL 144 hour cultures of E. coli JM109 (pAC-ispA(EC)-crtM(SA)-crtN(SA)/pUC-crtOx(SA)) were resuspended in 50 mL of dH₂O, KOH added to a final concentration of 10%, and samples incubated at 65° C. for 2 hours or at room temperature overnight. Insoluble material was pelleted by centrifugation and the supernatant extracted twice with 50 mL hexanes. The remaining, lower aqueous phase was kept and acetic acid added to pH 4. This acidified sample was then extracted with ethyl acetate, the pigment forming a precipitate between the phases that was recovered, dried under a stream of N₂ gas and stored at −20° C.

### Results

### Identification of C30 Carotenoid Genes

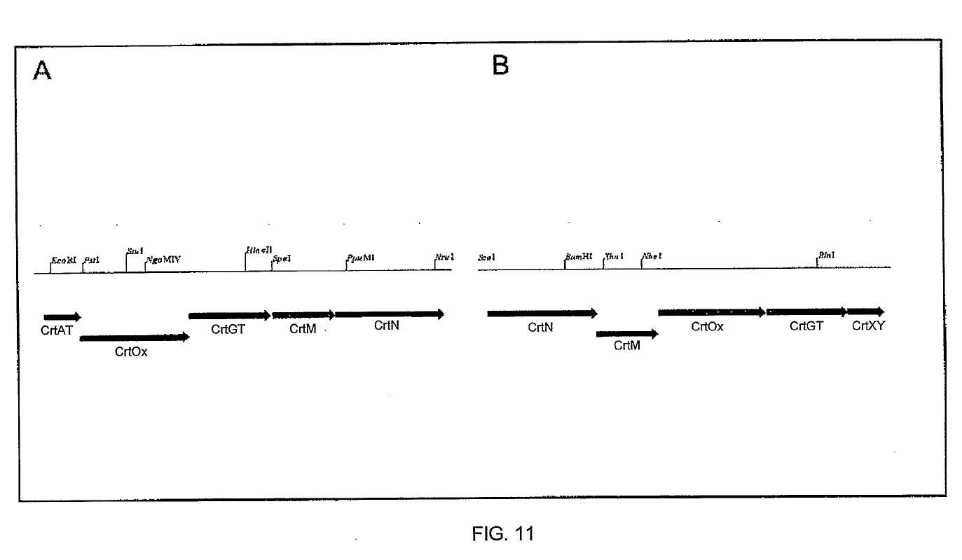

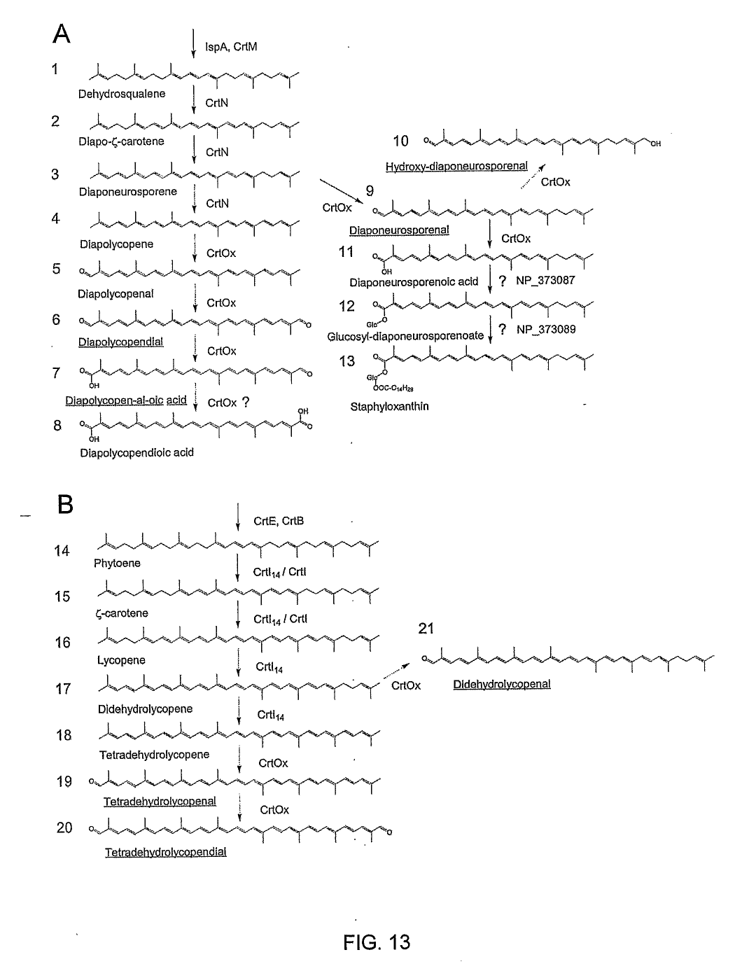

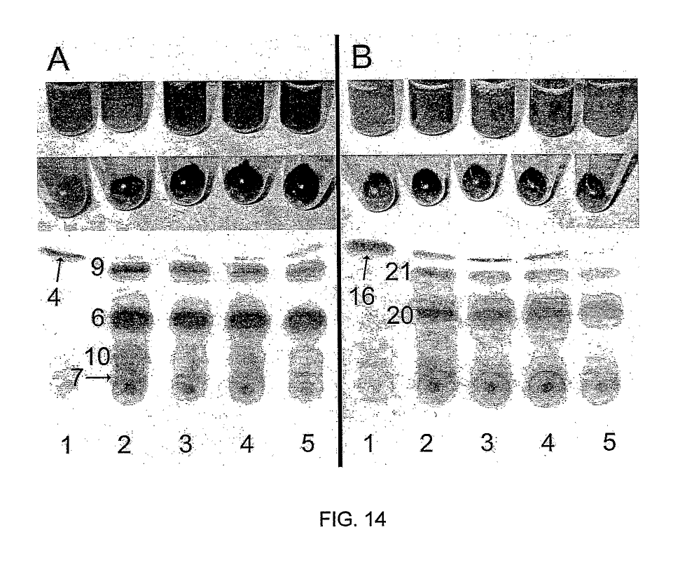

Analysis of the genome region of S. aureus where CrtM and CrtN are present indicated the presence of a number of closely spaced genes in the same orientation, typical of a microbial operon structure. The genes included a homolog of CrtN (CrtOx; 25% identity, 48% similarity), a putative glycosyl transferase (CrtGT), and an additional short ORF with no homology to known proteins. This short ORF may encode a putative 4,4′-diapocarotene acyl transferase (designated CrtAT). As previous results with mutants of C30 carotenoid producing strains indicated that reactions that generate additional double bonds and carboxyl termini are enzymatically linked, it was proposed that CrtOx is a dual function desaturase/oxygenase enzyme. Based on its homology to known glycosyl transferases, the CrtGT gene was proposed to be a glycosyl transferase that produces glycosyl ester carotenoids. A BLAST search did not reveal any homologous sequences for the short ORF. The structure of the operon can be seen in FIG. 11A and suggested these genes may be involved in the biosyiithesis of staphyloxanthin (structure 13; FIG. 13), the glycosylated, acylated major carotenoid of S. aureus (Marshall and Wilmoth, J. Bacteriol., 147: 900-13 (1981)). In order to functionally characterize these genes they were subcloned into the over-expression vector pUCMod for expression in E. coli. E. coli strains harboring plasmids designed to express CrtGT or CrtAT grew extremely slowly with an unusual, transparent colony morphology on agar plates. E. coli JM109 harboring pUCMod-crtOx(SA) did not demonstrate this growth inhibition. When pUCMod-crtOx(SA) was electrotransformed into strain JM109 harboring the plasmid pAC-ispA(EC)-crtM(SA)-crtN(SA) (producing the C₃₀ carotenoid diapolycopene), cells with a deep red phenotype were produced (FIG. 14A) indicating a change in carotenoid production when compared to the yellow-orange cells of the background strain.

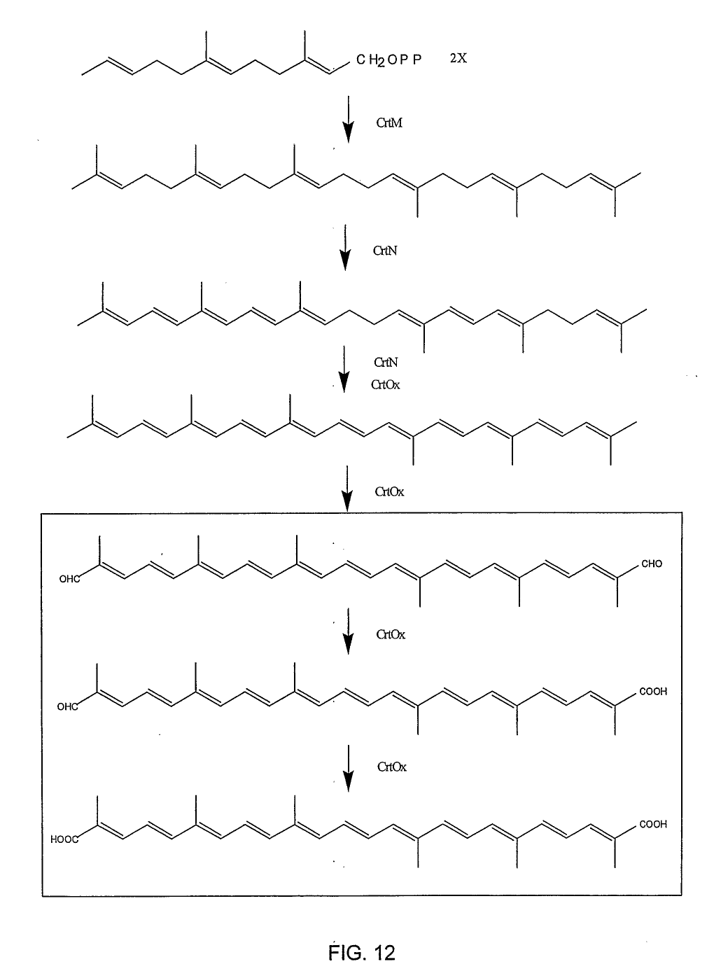

BLAST searches against the NCBI GenBank® database revealed homologues of each of these genes are present in the genome of Oceanobacillus iheyensis. A similar operon structure is present in this organism although the gene arrangement is different (see FIG. 11B). The proposed engineered biosynthetic pathway for these enzymes is provided in FIG. 12.

### Cloning and Expression of Novel Carotenoid Genes

Production of novel C30 carotenoids was attempted by co-expressing the plasmids pAC-ispA(EC)-crtM(SA)-crtN(SA) and pUC-crtOx(SA) in E. coli strain JM109. The crtOx (putative diapocarotene oxidase) gene was expressed on the high copy number plasmid pUCMod, and the remaining carotenoid pathway genes on the low copy number plasmid pACMod in order to direct metabolic flux towards more polar carotenoid end products. This strain, along with a control strain harboring pUCMod in place of pUC-crtOx(SA), were cultured in LB medium supplemented with carbenicillin and chloramphenicol for 24 hours at 37° C. and spun at 300 RPM. The carotenoids were extracted with acetone. E. coli expressing pAC-ispA(EC)-crtM(SA)-crtN(SA) and pUC-crtOx(SA) produced a distinctive violet to deep red phenotype when compared to the control strain. Analysis of the carotenoids of this strain by normal phase silica-gel TLC using a ethyl acetate:hexane 1:3 mobile phase revealed the presence of a number of novel, polar carotenoids when compared to the control strain. Cloning and expression in E. coli of the two other carotenoid ORF's crtGT and crtAT resulted in a pleiotrophic phenotype. E. coli clones constitutively expressing crtGT or crtAT on pUCmod were negatively affected in cell growth and exhibited an aberrant colony morphology (shiny, small colonies), indicating that both genes encode enzymes with broad substrate specificity that act on substrates other than carotenoid too, e.g. membrane lipids.

Previous results have indicated that medium and culture conditions can considerably influence the yield and product distribution of recombinantly produced carotenoids. The E. coli (pAC-ispA(EC)-crtM(SA)-crtN(SA) and pUC-crtOx(SA)) strain producing novel carotenoids was therefore cultured in different media at different temperatures to optimize production. Both LBG and TB medium had considerably higher carotenoid production than LB medium, and overall carotenoid production in LBG medium was highest. In both cases, higher carotenoid production was observed at 30° C. compared to 37° C. Different color phenotypes were observed for cell pellets—dark orange/red in TB medium and dark purple in LBG medium. TLC analysis indicated the presence of similar product profiles but different product distributions—a violet pigment being the dominant product in LBG medium with higher accumulation of less polar precursors in TB medium. For remaining experiments, recombinant strains were cultured in LBG medium at 30° C., 300 RPM.

Carotenoid production was also analysed from strains harboring O. iheyensis genes in place of the pACMod vector, pUCMod vector or both. In each case, the carotenoid products generated were similar but different product distributions were present. In general, the genes from S. aureus appeared to produce higher yields of the most polar pigments.

### Initial Identification of Novel Carotenoid Products

In order to structurally characterize the obtained carotenoid products, a 500 mL culture was grown under optimized conditions. The carotenoids were extracted into acetone and then partitioned into two solvent phases (less polar hexanes and more polar ethyl acetate). The products were separated by open column silica gel chromatography. Each solvent partition yielded a number of different carotenoid fractions of increasing polarity that were visualized by TLC. In total, five unique carotenoid fractions were identified and analyzed by LC-MS. The major product of the more polar ethyl acetate solvent fraction was the strong red/violet compound 3, which was found to have a parent mass of 429.1 and a fragmentation pattern consistent with the fully desaturated C30 dialdehyde diapocarotenoid 4,4′-Diapocarotene-4,4′-dial. In addition, the more polar violet compound 5 was found to have a parent mass of 445.2 and a fragmentation pattern consistent with the structure 4,4′-Diapocarotene-4-al, 4′-oic acid. The remaining compounds have parent masses consistent with mono- and di-aldehyde precursors of varying carotenoid backbone desaturation states (Table 4). The presence of compound 5 strongly suggested that a CrtOx catalyzed, non-specific reaction from terminal aldehyde to carboxyl function occurs at a relatively slow rate.

TABLE 4
Parent masses and proposed structures of carotenoid fractions.

| Carotenoid | | |
|---|---|---|
| fraction | Parent Mass | Initial Compound Name |
| 1 | 417.2 | 4,4′-diapo-ζ-carotene-al |
| 2 | 497.2 | Possible C35 carotenoid |
| 3 | 429.1 | 4,4′-diapo-lycopene-dial |
| 4 | 433.2 | 4,4′-diapo-ζcarotene-dial |
| 5 | 445.2 | 4,4′-diapo-lycopene-al-oic acid |

A time course experiment was performed under the optimized culture conditions to detect additional dicarboxylic acid carotenoid products and to improve the production of more polar C30 carotenoids, in particular dicarboxylic acid derivatives. Pellets from each time sample were extracted with acetone, and the solvent accessible carotenoids characterized by HPLC analysis. It was also found that significant levels of pigments that resisted extraction in acetone were present in 48 hour and higher samples. Furthermore, subsequent extractions of the acetone extract cell pellets indicated that this compound could not be extracted using common laboratory organic solvents (chloroform, methanol, ethanol, hexanes, petroleum ether, ethyl acetate, and DMSO).

Initial experiments indicated that the non-solvent accessible pigment produced was soluble in low concentrations of aqueous alkali salts such as NaOH or KOH. It was also found that this compound precipitated from aqueous solutions below pH 6.5, but the violet precipitate formed was not soluble in organic solvents. These physical properties are consistent with those of a short chain dicarboxylic acid carotenoid such as norbixin, which is soluble in aqueous solutions only as a salt.

### Example 11

### Synthesis of C30 and C40 Carotenoids

Nucleic acid encoding S. aureus CrtOx was expressed in E. coli cells designed to synthesize linear C30 or C40 carotenoids. When expressed in engineered E. coli cells synthesizing linear C30 carotenoids, novel polar carotenoid products were generated, identified as aldehyde and carboxylic acid C30 carotenoid derivatives. The most abundant product in this engineered pathway was the fully desaturated C30 dialdehyde carotenoid 4,4′-diapolycopen-4,4′-dial. Very low carotenoid yields were observed when CrtOx was complemented with the C40 carotenoid lycopene pathway. But extension of an in vitro evolved pathway of the fully desaturated 2,4,2′,4′-tetradehydrolycopene produced the structurally novel, fully desaturated C40 dialdehyde carotenoid 2,4,2′,4′-tetradehydrolycopendial. Directed evolution of CrtOx(SA) by error-prone PCR resulted in a number of variants with higher activity on C40 carotenoid substrates and improved product profiles. These results demonstrate that new biosynthetic routes can be used to produce highly polar carotenoids with unique spectral properties desirable for a number of industrial and pharmaceutical applications.

### Methods and Materials

### Plasmid Construction

The construction of the plasmids pAC-ispA(EC)-crtM(SA)-crtN(SA), pAC-crtE(EU)-crtB(EU)-crtI(EU), and pAC-crtE(EU)-crtB(EU)-crtI₁₄ producing diapolycopene, lycopene, and 2,4,2′,4′-tetradehydrolycopene, respectively, as well as pUCMod-crtOx(SA), pUCMod-crtGT(SA), and pUCMod-crtAT(SA) were made as described herein.

For error-prone PCR mutagenesis, the crtOx(SA) gene in pUCMod was amplified with the PCR primers (5′-CCGACTGGAAAGCGGG-3′, SEQ ID NO:14; and 5′-ACAAGCCCGTCAGGG-3, SEQ ID NO:15) flanking the gene and promoter. The PCR reaction mix consisted of 1× Promega Mg²⁺ free thermophilic buffer (Promega, Madison, Wis.), 10 ng/mL template plasmid, 1 μM of each primer, 5 Units Taq DNA polymerase, 0.3 mM dNTP mix. MgCl₂ and MnCl₂ were added to a final total salt concentration of 2 mM, and separate reactions were performed with 0.2, 0.1, 0.05, and 0.025 mM final concentrations of MnCl₂. PCR was carried out with a program of 95° C. for 4 minutes followed by 32 cycles of 94° C. for 1 minute, 50° C. for 1 minute, and 72° C. for 1 minute and finally 72° C. for 7 minutes. The PCR products were purified using a QIAquick gel extraction kit (Qiagen, Valencia, Calif.), combined and digested with the restriction enzymes XbaI and NotI. The DNA fragments were ligated into the corresponding sites of the pUCmod vector (Schmidt-Dannert et al., Nat. Biotechnol., 18:750-3 (2000)) and electrotransformed into competent E. coli JM109 harboring pAC-crtE(EU)-crtB(EU)-crtI₁₄. Transformants were plated on LB agar plates supplemented with 100 μg/mL carbenicillin and 50 μg/mL chloramphenicol. After 18 hours of incubation at 30° C. in the dark, colonies were replicated using a nitrocellulose membrane and transferred onto fresh LB plates containing the same antibiotics. Colonies were screened visually for color variants after an additional 24 hour incubation at room temperature. Mutations in the S. aureus crtOx sequence were confirmed by DNA sequencing.

### Carotenoid Production and Extraction

For HPLC and HPLC-mass spectrometry analysis, 100 mL cultures were grown in LB medium supplemented with 0.5% glycerol, 100 μg/mL carbenicillin, and 50 μg/mL chloramphenicol at 30° C. for 24 hours, and cells harvested by centrifugation (30 minutes, 4000×g, 4° C.). Carotenoid extraction was performed as described elsewhere (Lee et al., Chem. Biol., 10:453-62 (2003)). Briefly, 5 mL of acetone was added to cell pellets, and samples were incubated in a sonicating water bath at 4° C. for 30 minutes, followed by centrifugation (20 minutes, 4000×g, 4° C.) to remove cell debris. Extractions with acetone were repeated until no visible pigment remained, and the supernatants were pooled. Pooled extracts were dried down completely under a stream of N₂ gas and resuspended in 5 mL of ethyl acetate. Carotenoids were two-phase extracted with 10 mL 5M NaCl, and the solvent phase was recovered, dried down, and resuspended in hexane or ethyl acetate.

### TLC Analysis

Crude and purified C30 and C40 carotenoid extracts were initially analyzed by thin-layer choromatography with Whatman normal phase silica gel 60 plates developed using hexane:ethyl acetate (3:1).

### HPLC and HPLC-Mass Spectrometry Analysis of Carotenoids

HPLC separation was performed using a Zorbax 300SB-C18 column (4.6×150 mm, 2.5 μm; Agilent technologies, Palo Alto, Calif.) at a flow rate of 1 mL min⁻¹ using an Agilent 1100 HPLC system equipped with a photodiode array detector. For carotenoid separations, the mobile phase consisted of dH₂O:acetonitrile (30:70) for 0-5 minutes followed by a gradient to 100% acetonitrile at 45 minutes. Mass spectrometry was performed under the same conditions as HPLC analysis. Mass spectra were monitored in a mass range of m/z 200-1000 on a LCQ mass spectrophotometer equipped with an atmosphere pressure chemical ionization (APCI) interface (Thermo Finnigan, USA) as described elsewhere (Lee et al., Chem. Biol., 10:453-62 (2003)).

### Results

### Further Characterization of Novel C₃₀ Carotenoids

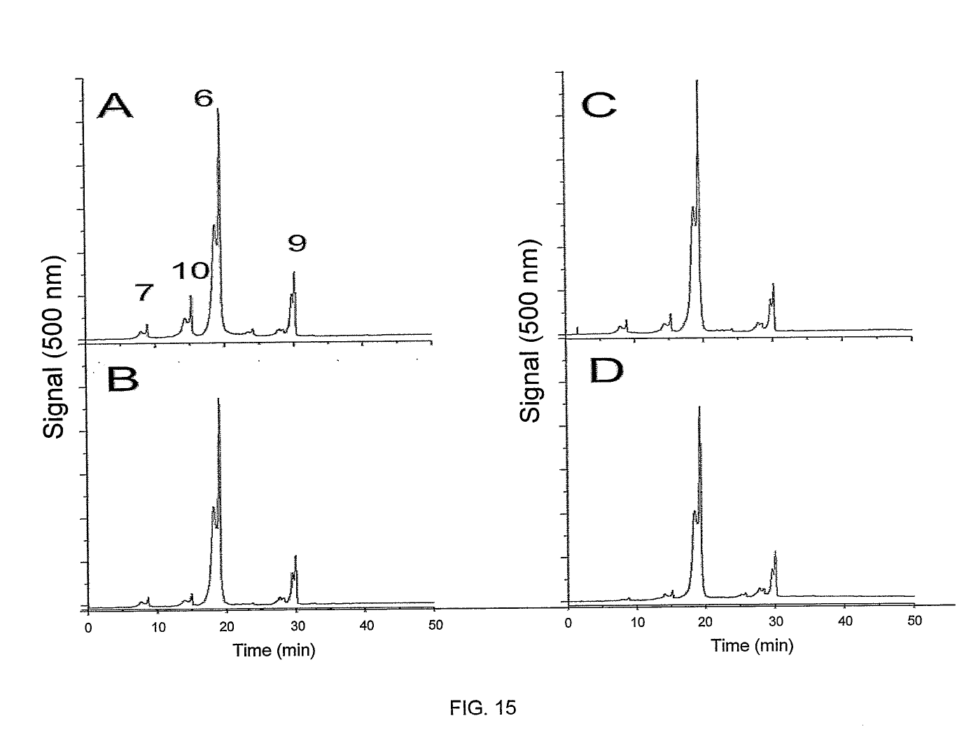

The following experiments were performed to extend the work provided in Example 10. For analysis of carotenoids, E. coli strain JM109 harboring pAC-ispA(EC)-crtM(SA)-crtN(SA) and pUC-crtOx(SA) was cultured in LB medium supplemented with glycerol at 30° C. Initial analysis of carotenoid extracts by TLC (FIG. 14) indicated that a number of novel carotenoids were present compared to a control strain JM109 harboring pAC-ispA(EC)-crtM(SA)-crtN(SA) and pUCMod vector without insert DNA. HPLC analysis indicated the presence of a number of additional polar peaks (FIG. 15A). These were analyzed by mass spectrometry and by a combination of HPLC retention times, UV-Vis fine spectra and Mass-spectra the major peaks were assigned structures in FIG. 13. The properties of the novel carotenoids are summarized in Table 5. The major product was a violet compound with a [M]⁺ of m/z 429.0 assigned as 4,4′-diapolycopen-4,4′-dial. Characteristic mass fragments of two aldehyde functions were observed (M-18, M-28, M-18-18, M-18-28) and characteristic carotenoid extrusion losses of toluene (M-92) and xylene (M-106). The major violet carotenoid reacted very rapidly with NaBH₄, producing a more polar, yellow-orange compound by TLC analysis as a result of the reduction of terminal aldehyde groups to hydroxyl groups. A less polar peak with a [M]⁺ of m/z 417.1 consistent with a mono-aldehyde derivative of diaponeurosporene (FIG. 15A, peak 9) was also observed with mass fragments characteristic of a single aldehyde group (M-18, M-28). Reaction with NaBH₄ rapidly generated a more polar, yellow product by TLC with an Rf consistent with a carotenoid monoalcohol. These results indicate that the crtOx enzyme catalyzes the addition of one or more aldehyde groups to C30 carotenoid terminal methyl groups and is likely responsible for the synthesis of the mono-aldehyde intermediate observed in the biosynthesis of staphyloxanthin (Marshall and Wilmoth, J. Bacteriol., 147:914-9 (1981)). These results also confirm that the enzyme encoded by the crtOx gene is an oxygenase, namely diapocarotenal synthase.

TABLE 5
Properties of carotenoids.

| | | | | | UV-Vis Maxima |
|---|---|---|---|---|---|
| Structure | | Exact | Observed | Observed | [shoulder] |
| (FIG. 13) | Compound | Mass | [M]⁺ (m/z) | Fragments | (in acetonitrile) |
| 6 | Diapolycopenedial | 428.27 | 429.0 | M-18, M-28, | 508, [536] |
| | | | | M-36, M-46, | |
| | | | | M-92, M-106 | |
| 7 | Diapolycopenal-oic acid | 444.27 | 445.0 | M-18, M-36, | 515, [539] |
| | | | | M-110 | |
| 9 | Diaponeurosporenal | 416.31 | 417.1 | M-18, M-28, | 469, [490] |
| | | | | M-92, M-106 | |
| 10 | Hydroxy- | 432.3 | 433.1 | M-2, M-18, | 480, [500] |
| | Diaponeurosporenal | | | M-28, M-36, | |
| | | | | M-92, M-106 | |
| 19 | Tetradehydrolycopenal | 546.39 | 547.2 | M-18, M-92, | 521, [552] |
| | | | | M-106 | |
| 20 | Tetradehydrolycopendial | 560.37 | 561.1 | M-18, M-28, | 537, [563] |
| | | | | M-36, M-92, | |
| | | | | M-106 | |
| 21 | Didehydrolycopenal | 548.4 | 549.2 | M-18, M-28, | 513, [541] |
| | | | | M-92, M-106 | |

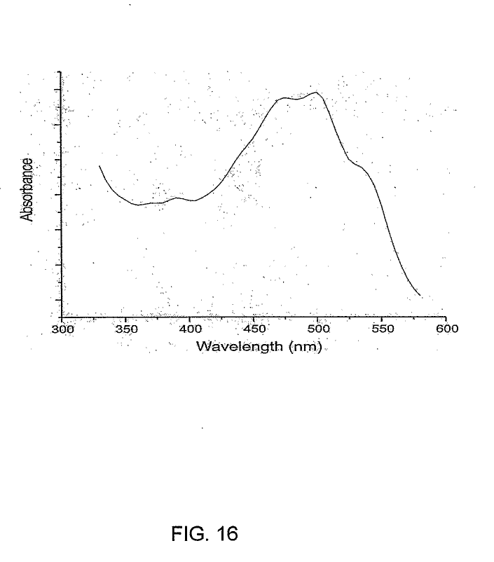

In addition to the major product 4,4′-diapolycopen-4,4′-dial, two more polar peaks were observed by HPLC analysis, suggesting products with additional oxygen functions are present. Although present in relatively low yields, parent masses could be obtained for both compounds, and structures putatively assigned. The least polar of these (FIG. 15A, peak 10) has a parent mass [M]⁺ of m/z 433.1, and has characteristic fragments of M-2 and M-18, and was assigned as hydroxy-4,4′-diaponeurosporenal. Reaction with NaBH₄ was rapid and produced a more polar, yellow product by TLC indicating reduction to a carotenoid dialcohol. The lack of UV-Vis fine structure observed for this compound (Table 5) suggests that the aldehyde function is located adjacent to the conjugated double bond system, and this compound could therefore be assigned as 4′-hydroxyl-4,4′-diaponeurosporen-4-al (FIG. 13, compound 10). The most polar peak (FIG. 15A, peak 7) with a [M]⁺ of m/z 445.0 and a very strong fragment at M-18 and an additional fragment at M-36 was assigned as 4,4′-diapolycopen-4-al-4′-oic acid. Although present in relatively low yields, the presence of this compound suggests that CrtOx catalyses both the oxidation of C30 carotenoids to aldehydes and the further oxidation of these aldehyde groups to carboxylic acids. The expected final product of this engineered pathway, 4,4′-diapolycopen-4,4′-dioic acid, was not observed in acetone extracts, and increasing culture times of 48, 64, and 96 hours yielded only similar product profiles by HPLC analysis. However, cell pellets of these cultures after acetone extraction demonstrated that levels of a red pigment not accessible by acetone extraction increased over time. This pigment could not by extracted with a range of organic solvents but could be solubilized by the addition of 1% aqueous KOH to cell pellets followed by stirring at room temperature for 2 hours. This is consistent with the chemical properties of the plant C24 dicarboxylic acid carotenoid norbixin (Bouvier et al., Science, 300:2089-91 (2003)), which forms a soluble potassium salt in aqueous KOH. On the addition of acetic acid to pH 5, an insoluble precipitate formed which was not soluble in a number of organic solvents tested with the exception of DMSO. A UV-Vis spectral scan of this compound in KOH is provided in FIG. 16. While enriched samples were obtained, analytical grade preparations of this compound were not apparently due to the lack of solubility. However, the known biochemical pathway and physical properties of the pigment strongly suggest a diapocarotene-dioic acid.

C40 Carotenoids Produced by Complementation of CrtOx with the In Vitro-Evolved Tetradehydrolycopene Pathway

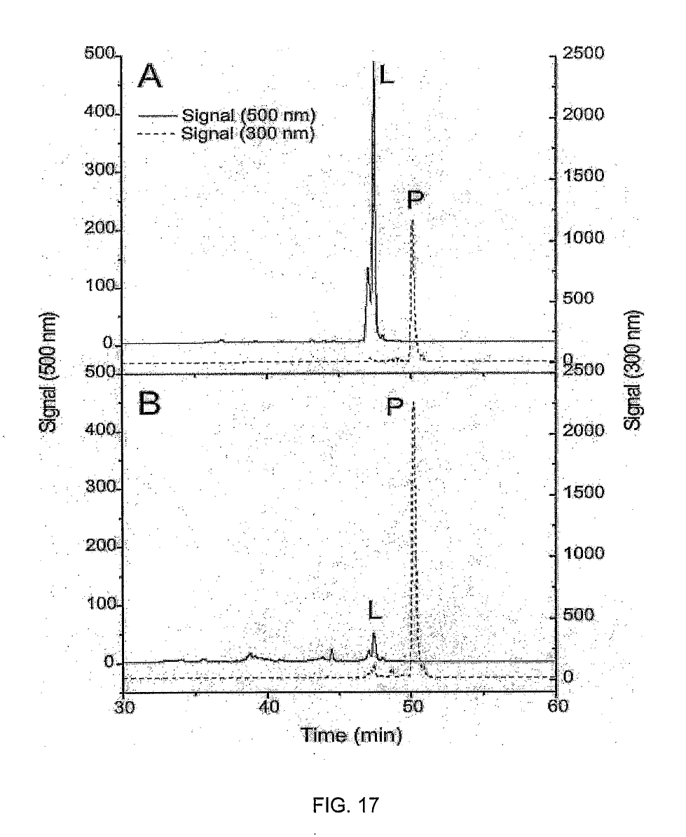

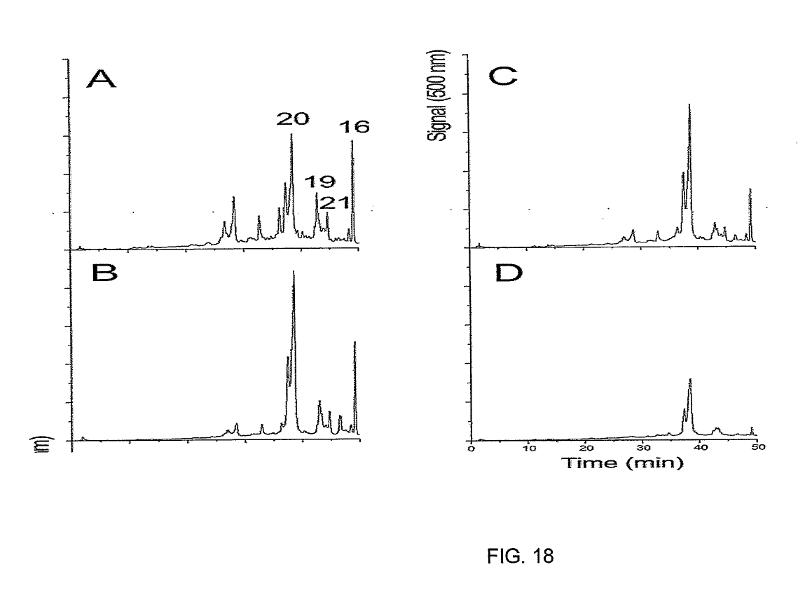

When E. coli JM109 harboring the plasmid pAC-crtE(EU)-crtB(EU)-crtI(EU) necessary for C40 carotenoid lycopene synthesis was electrotransformed with pUC-crtOx(SA), very low levels of more polar carotenoids were observed by TLC when compared to a control strain transformed with pUCMod. HPLC analysis of carotenoid extracts of E. coli strain JM109 harboring pAC-crtE(EU)-crtB(EU)-crtI(EU) with pUCMod or pUC-crtOx(SA) (FIG. 17) indicated that the presence of CrtOx significantly reduced the yield of lycopene and increased the accumulation of the precursor molecule phytoene. Although very low levels of more polar products were observed, these results indicate that lycopene is a poor substrate for CrtOx and that an enzyme interaction may be disrupting desaturation by CrtI. The complementation of the in vitro-evolved C40 carotenoid 2,4,2′,4′-tetradehydrolycopene pathway with a number of carotenoid modifying enzymes in recombinant E. coli is described herein and elsewhere (Lee et al., Chem. Biol., 10:453-62 (2003)). When E. coli JM109 harboring the plasmid pAC-crtE(EU)-crtB(EU)-crtI₁₄ necessary for tetradehydrolycopene synthesis was electrotransformed with pUC-crtOx(SA), cells with a deep red color phenotype were produced when compared to the pink/red color of the background strain with pUCMod vector without insert. HPLC analysis (FIG. 18A) indicated that a number of new polar products were present. In order to structurally characterize these carotenoids, they were analyzed by HPLC-mass spectrometry. The assigned structures of the major peaks on the HPLC chromatogram shown in FIG. 13 were determined by a combination of HPLC retention times, UV-Vis spectra, and Mass spectra summarized in Table 5. The major product (FIG. 18A, peak 20), with a parent mass [M]⁺ of m/z 561.1 and mass fragments characteristic of two aldehyde functions (M-18, M-28, M-18-18) and characteristic carotenoid extrusion losses of toluene (M-92) and xylene (M-106), was identified as the fully desaturated C40 dialdehyde 2,4,2′,4′-tetradehydrolycopendial. However, considerable levels of the C40 biosynthesis pathway precursor lycopene were also observed (FIG. 18A, peak 16). This suggests that although CrtOx is active on more desaturated C40 carotenoid substrates, it has little activity on pathway precursors such as lycopene. Two additional less polar molecules could be identified as mono-aldehyde derivatives. The least polar of these (FIG. 18A, peak 21) with a [M]⁺ of m/z 549.2 was assigned as 2,4-didehydrolycopenal; and the higher yield, more polar peak (FIG. 18A, peak 19) with a [M]⁺ of m/z 547.2 was assigned as 2,4,2′,4′-tetradehydrolycopenal. Both had the characteristic mass fragments of one aldehyde function (M-18, M-28) and characteristic carotenoid extrusion losses of toluene (M-92) and xylene (M-106). No peaks corresponding to aldehyde derivatives of lycopene were present. Although additional highly polar compounds can be observed in the HPLC chromatogram, these were relatively low yield and molecular structures could not be positively identified by mass spectrometry. These may represent low yields of mono- or dicarboxylic-acids or non-specific pathway derivatives.

### Construction of In Vitro Evolution Libraries and Isolation and Sequence of Mutants

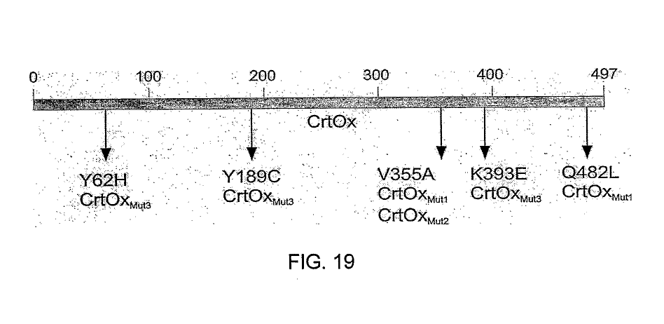

In order to alter the product profile of the C40 carotenoids produced by complementation with CrtOx and improve the relative yields of oxygenated C40 carotenoids, an error-prone PCR mutagenesis library of CrtOx was constructed and electrotransformed into E. coli strain JM109 harboring the plasmid pAC-crtE(EU)-crtB(EU)-crtI₁₄ necessary for 2,4,2′,4′-tetradehydrolycopene production. Colonies with altered carotenoid production were identified by color screening. Screening of about 3000 colonies by this method yielded three mutants with altered cell pigment phenotypes designated CrtOx(SA)_{Mut1}, CrtOx(SA)_{Mut2} and CrtOx(SA)_{Mut3}. These colonies had deep purple (CrtOx(SA)_{Mut1}, CrtOx(SA)_{Mut2}) and blue/grey (CrtOx(SA)_{Mut3}) color phenotypes (FIG. 14B). The DNA sequences of the inserts of these plasmids were determined, and plasmids re-transformed into E. coli cells harboring pAC-crtE(EU)-crtB(EU)-crtI₁₄, confirming the color phenotypes observed. The mutants were also electrotransformed into E. coli JM109 harboring pAC-ispA(EC)-crtM(SA)-crtN(SA) to test the activity of these mutants against C30 carotenoid substrates (FIG. 14A). The amino acid sequence changes observed in the mutant CrtOx genes are provided in FIG. 19.

Carotenoid Analysis of In Vitro-Evolved CrtOx Mutants Complemented with C30 and C40 Pathways

HPLC chromatograms of the carotenoid products of the in vitro-evolved CrtOx genes coexpressed with the recombinant 2,4,2′,4′-tetradehydrolycopene pathway are shown in FIG. 18B-D. Based on HPLC retention times and UV-Vis spectra, it appeared that the mutant enzymes produced no significant amounts of new products. However, considerable alterations in the product profiles were detected, consistent with the altered color phenotypes observed by screening (FIG. 14B). CrtOx(SA)_{Mut1} and CrtOx(SA)_{Mut2}, as expected from the similar amino acid sequence and colony color phenotype of these mutants, have similar overall product profiles. Both have higher yields of 2,4,2′,4′-tetradehydrolycopendial (CrtOx_{Mut1}, 57% increase; CrtOx_{Mut2}, 18% increase) and lower yields of the more polar, unidentified products. In addition, a significant decrease in the relative yield of the precursor lycopene was also observed. This is likely to be partly responsible for the altered color phenotypes observed by screening. By peak integration of spectra at 500 nm, wild-type CrtOx(SA) produces a ratio of 2.6:1 (2,4,2′,4′-tetradehydrolycopendial:lycopene) whereas CrtOx(SA)_{Mut1} has a ratio of 4.8:1 and CrtOx(SA)_{Mut2} 6.4:1. Finally, CrtOx(SA)_{Mut3} exhibited the most dramatic change in product spectrum accordant with the greater number of amino acid changes observed. Although it has the lowest yield of 2,4,2′,4;-tetradehydrolycopendial (41% decrease compared to CrtOx(SA) wild type), the precursor compound lycopene is almost completely absent compared to 2,4,2′,4;-tetradehydrolycopendial with a ratio of 21:1, and significant peaks of the more polar, unidentified compounds observed are not present. By integration of all carotenoid peaks observed at 500 nm, 2,4,2′,4;-tetradehydrolycopendial represents 84.5% of the total carotenoids detected in this strain.

In contrast, the in-vitro evolved CrtOx enzymes appeared to have a less dramatic effect on the product profiles of the C30 diapolycopene pathway (FIG. 15B-D). However, a considerable decrease in the accumulation of the more polar products can be observed (FIG. 15B-D, peaks 7 and 10). These results suggest that the mutations can have some influence on the putative carboxylic acid synthesis function of the enzyme on C30 carotenoid substrates.

Taken together, the results provided herein demonstrate that the crtOx enzyme can catalyze the biosynthesis of both the aldehyde and carboxylic acid intermediates in staphyloxanthin biosynthesis. The major product observed in recombinant E. coli engineered to express ispA(EC), crtM(SA), crtN(SA), and crtOx(SA) was a dialdehyde derivative of the fully desaturated C30 carotenoid diapolycopene, which is in contrast to staphyloxanthin, a diaponeurosporene derivative oxygenated at only one terminus. This product is likely the result of the engineered E. coli pathway in which all enzymes are constitutively expressed with CrtOx being expressed from a high copy number plasmid (e.g., pUCMod) and the remaining genes being expressed from a low copy-number plasmid (e.g., pACMod). This may increase the pathway flux to more oxygenated products and thus a di-aldehyde carotenoid derivative is formed.

As CrtOx is homologous to CrtN and other carotenoid desaturases, it is possible that it retains some desaturase activity. Expression of CrtOx and CrtM, in the absence of CrtN, however, failed to produce pigmented carotenoids. The amino acid sequence relatedness of CrtN and CrtOx suggests evolution via a gene duplication event and subsequent functional differentiation.

In addition, CrtOx was a relatively promiscuous enzyme, readily able to accept C40 carotenoid substrates. Initial experiments by complementing CrtOx with the genes necessary for synthesis of the C40 carotenoid lycopene (FIG. 13, structure 16) resulted in a significant reduction in carotenoid yield when compared to a control. When combined with the in vitro-evolved 2,4,2′,4′-tetradehydrolycopene pathway, CrtOx catalyzed the synthesis of highly desaturated mono- and di-aldehyde C40 carotenoids. The engineered 2,4,2′,4′-tetradehydrolycopene pathway also accumulates significant levels of the precursor lycopene, and this was also observed with the addition of CrtOx. This accumulation, and the lack of observed oxygenated lycopene derivatives, indicates that CrtOx preferentially accepts more desaturated substrates. The lack of carotenoid production and desaturation activity observed when CrtOx was co-expressed with the lycopene biosynthesis pathway may be the result of the formation of a disrupted carotenogenic enzyme complex. The major product of CrtOx activity on the 2,4,2′,4′-tetradehydrolycopene biosynthesis pathway was identified as the deep violet dialdehyde derivative 2,4,2′,4′-tetradehydrolycopendial. Although a number of more polar peaks were observed on HPLC analysis, they were not positively identified. Based on the results of the engineered C30 pathway, these may represent carboxylic acid derivatives.

Color screening of CrtOx error-prone PCR libraries with the engineered 2,4,2′,4′-tetradehydrolycopene pathway yielded three clones with an altered color phenotype. Sequencing revealed each clone contained a unique pattern of mutations although CrtOx(SA)_{Mut1} CrtOx(SA)_{Mut2} share an amino acid change. Although HPLC analysis of the carotenoid profiles of the mutant strains indicated no novel products were observed, considerable changes in product distribution were observed. These changes are responsible for the altered color phenotypes observed. All of these mutants have relatively reduced yields of the pathway precursor lycopene, which again suggests some interaction of these heterologous enzymes is taking place. Mutant CrtOx(SA)_{Mut3} has the most significant change in product profile and although overall yield is lower, 2,4,2′,4′-tetradehydrolycopendial in produced in considerable excess over other carotenoids detected. Co-expression of the CrtOx variants with the C30 carotenoid biosynthesis pathway yielded similar carotenoid product profiles to the wild-type CrtOx clone. However, a reduced accumulation of the more polar carboxylic acid products observed in these samples, along with the C40 pathway, suggests these mutations may compromise the aldehyde oxidase function of the enzyme. This also indicates that these highly polar carotenoid products are the result of enzymatic activity of wild-type CrtOx and not non-specific in vivo activity.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A microorganism comprising exogenous nucleic acid encoding a polypeptide having a carotenoid oxygenase activity, wherein said microorganism has a geranylgeranyl diphosphate (GGDP) synthase activity, a phytoene synthase activity, and a phytoene desaturase activity and produces detectable amounts of at least one compound selected from the group consisting of dialdehyde 2,4,2′,4′-tetradehydrolycopendial; 2,4-didehydrolycopenal; and 2,4,2′,4′-tetradehydrolycopenal.

2. The microorganism of claim 1, wherein said polypeptide having said carotenoid oxygenase activity is an S. aureus carotenoid oxygenase.

3. The microorganism of claim 1, wherein said polypeptide having said carotenoid oxygenase activity is an O. iheyensis carotenoid oxygenase.

4. The microorganism of claim 1, wherein said microorganism produces more 2,4,2′,4′-tetradehydrolycopendial than lycopene such that the ratio is greater than 3:1 2,4,2′,4′-tetradehydrolycopendial to lycopene.

5. The microorganism of claim 4, wherein said ratio is greater than 5:1.

6. The microorganism of claim 4, wherein said ratio is greater than 10:1.

7. The microorganism of claim 4, wherein said ratio is greater than 20:1.

8. The microorganism of claim 4, wherein said polypeptide having said carotenoid oxygenase activity is crtOx(SA)ₘᵤₜ₁ or crtOx(SA)ₘᵤₜ₂.

9. The microorganism of claim 4, wherein said polypeptide having said carotenoid oxygenase is crtOx(SA)ₘᵤₜ₃.

10. The microorganism of claim 1, wherein said microorganism comprising exogenous nucleic acid encoding a polypeptide having said geranylgeranyl diphosphate synthase activity, a polypeptide having said phytoene synthase activity, and a polypeptide having said phytoene desaturase activity.

11. The microorganism of claim 10, wherein said polypeptide having said geranylgeranyl diphosphate synthase activity is an E. uredovora geranylgeranyl diphosphate synthase.

12. The microorganism of claim 10, wherein said polypeptide having said phytoene synthase activity is an E. uredovora phytoene synthase.

13. The microorganism of claim 10, wherein said polypeptide having said phytoene desaturase activity is an E. uredovora phytoene desaturase.

14. The microorganism of claim 10, wherein said polypeptide having said phytoene desaturase activity is crtI₁₄.

15. The microorganism of claim 10, wherein said exogenous nucleic acid encoding said polypeptide having said geranylgeranyl diphosphate synthase activity, said polypeptide having said phytoene synthase activity, said polypeptide having said phytoene desaturase activity, and said polypeptide having said carotenoid oxygenase activity is located on a single nucleic acid molecule.

16. The microorganism of claim 10, wherein said exogenous nucleic acid encoding said polypeptide having carotenoid oxygenase activity is located on a nucleic acid molecule separate from said exogenous nucleic acid encoding said polypeptide having said geranylgeranyl diphosphate synthase activity, said polypeptide having said phytoene synthase activity, and said polypeptide having said phytoene desaturase activity.

17. The microorganism of claim 1, wherein said phytoene desaturase activity is capable of catalyzing production of a fully conjugated 3,4,3′,4′-tetradehydrolycopene.

18. The microorganism of claim 1, wherein said microorganism produces detectable amounts of dialdehyde 2,4,2′,4′-tetradehydrolycopendial.

19. The microorganism of claim 1, wherein said microorganism produces detectable amounts of 2,4-didehydrolycopenal.

20. The microorganism of claim 1, wherein said microorganism produces detectable amounts of 2,4,2′,4′-tetradehydrolycopenal.

21. The microorganism of claim 1, wherein said microorganism is E. coli or S. aureus.

22. A composition comprising a compound selected from the group consisting of dialdehyde 2,4,2′,4′-tetradehydrolycopendial; 2,4-didehydrolycopenal; and 2,4,2′,4′-tetradehydrolycopenal.

23. The composition of claim 22, wherein greater than 10 percent of said composition is said compound.

24. The composition of claim 22, wherein greater than 50 percent of said composition is said compound.

25. The composition of claim 22, wherein greater than 80 percent of said composition is said compound.

26. The composition of claim 22, wherein said composition is a food composition.

27. A method of making a compound selected from the group consisting of dialdehyde 2,4,2′,4′-tetradehydrolycopendial; 2,4-didehydrolycopenal; and 2,4,2′,4′-tetradehydrolycopenal, said method comprising culturing the microorganism of claim 1 under conditions wherein said microorganism produces said compound.

28. The method of claim 27, said method further comprising extracting said compound from said microorganism.

29. The method of claim 27, wherein said microorganism produces at least about 1 mg/L of said compound.

30. The method of claim 27, wherein said microorganism produces at least about 10 mg/L of said compound.

31. The method of claim 27, wherein said microorganism produces at least 100 mg/L of said compound.

32. An isolated nucleic acid molecule encoding a carotenoid oxygenase that, when expressed in a microorganism having a geranylgeranyl diphosphate synthase activity, a phytoene synthase activity, and a phytoene desaturase activity, results in said microorganism producing more 2,4,2′,4′-tetradehydrolycopendial than lycopene such that the ratio is greater than 3:1 2,4,2′,4′-tetradehydrolycopendial to lycopene.

33. The isolated nucleic acid molecule of claim 32, wherein said ratio is greater than 5:1.

34. The isolated nucleic acid molecule of claim 32, wherein said ratio is greater than 10:1.

35. The isolated nucleic acid molecule of claim 32, wherein said ratio is greater than 20:1.

36. The isolated nucleic acid molecule of claim 32, wherein said isolated nucleic acid molecule encodes crtOx(SA)ₘᵤₜ₁ or crtOx(SA)ₘᵤₜ₂.

37. The isolated nucleic acid molecule of claim 32, wherein said isolated nucleic acid molecule encodes crtOx(SA)ₘᵤₜ₃.

38. An E. coli microorganism comprising exogenous nucleic acid encoding a polypeptide having a carotenoid oxygenase activity, a polypeptide having a geranylgeranyl diphosphate (GGDP) synthase activity, a polypeptide having a phytoene synthase activity, and a polypeptide having a phytoene desaturase activity, wherein said microorganism produces detectable amounts of dialdehyde 2,4,2′,4′-tetradehydrolycopendial, wherein said polypeptide having said carotenoid oxygenase activity is crtOx(SA)ₘᵤₜ₁, and wherein said polypeptide having said phytoene desaturase activity is crtI₁₄.
